(19) United States
(12) Patent Application Publication
(10) Pub. No.: US 2025/0289810 A1
(43) Pub. Date: Sep. 18, 2025

(54) Compounds As NLRP3 Inflammasome Inhibitors And Their Use As Medicaments

(71) Applicant: MAM-AGE S.R.L., Bologna (IT)

(72) Inventors: Paolo PINTON, Ferrara (IT); Carlotta GIORGI, Ferrara (IT); Delia PRETI, Ferrara (IT); Sonia MISSIROLI, Ferrara (IT); Mariasole PERRONE, Ferrara (IT); Salvatore PACIFICO, Ferrara (IT); Valentina ALBANESE, Ferrara (IT)

(21) Appl. No.: 18/860,386

(22) PCT Filed: Apr. 27, 2023
(86) PCT No.: PCT/EP2023/061152

§ 371 Date: Oct. 25, 2024

(30) Foreign Application Priority Data
Apr. 27, 2022 (IT) 102022000008246
Apr. 27, 2022 (IT) 102022000008294
Apr. 27, 2022 (IT) 102022000008321

(51) Int. Cl. C07D 413/12; A61K 31/18; A61K 31/40; A61K 31/445; A61K 31/4725; A61K 31/496; A61K 31/5377; C07C 311/19; C07C 311/42; C07D 207/48; C07D 211/96; C07D 217/26; C07D 401/12

(52) U.S. Cl. CPC C07D 413/12; A61K 31/18; A61K 31/40; A61K 31/445; A61K 31/4725; A61K 31/496; A61K 31/5377; C07C 311/19; C07C 311/42; C07D 207/48; C07D 211/96; C07D 217/26; C07D 401/12

(57) ABSTRACT

The present invention relates to an hexahydro-s-indacene compound of Formula (I) or its pharmaceutically acceptable salt wherein A is selected from the group consisting of (A1), (A2) and (A3) as defined in the claims. The compounds of the invention are medicaments for use in the treatment of immunopathologies related to the NLRP3 inflammasome, such as for example cancer, metabolic disorders, migraine, wound repair, neurodegenerative diseases and autoimmune diseases. The hexahydro-s-indacene compound of the invention is a selective inhibitor of the activation of the NL-RP3 inflammasome and, as such, is capable of reducing the NLRP3-mediated production of interleukin-1β and interleukin 18 in mammalian cells and tissues.

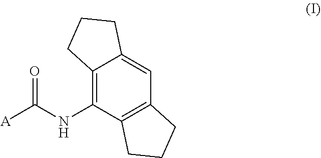

### BACKGROUND OF THE INVENTION

The present invention relates to hexahydro-s-indacene compounds, their pharmaceutically acceptable salts and their use as a medicament. Specifically, the invention provides selective inhibitors of the activation of NLRP3 inflammasome, which are able to reduce the NLRP3-mediated production of interleukin-1β (IL-1β) and interleukin-18 (IL-18) in mammalian cells and tissues. Therefore, the compounds of the invention can be used for the treatment of pathologies related to the overactivation of NLRP3 inflammasome resulting in overproduction of IL-1β and IL-18, such as for example cancer, metabolic disorders, neurodegenerative diseases and auto-immune diseases.

### STATE OF THE ART

The NLRP3 inflammasome is a multimeric protein complex formed by Nod like-receptor family protein containing a pyrin domain 3 (NLRP3), the adaptor apoptosis-associated speck-like protein (ASC), and the effector pro-caspase 1. Once activated, NLRP3 oligomerizes and interacts with ASC through its N-terminal pyrin domain (PYD), and then, ASC recruits and binds pro-caspase-1 via their shared domain CARD (caspase activation and recruitment domain), inducing autoproteolytic caspase-1 activation. Caspase-1, in turn, induces the maturation and release of the inflammatory cytokines interleukin-1 beta (IL-1β) and interleukin-18 (IL-18) (S. Missiroli et al. Cell Death Dis 2018; 9:329).

NLRP3 inflammasome activation requires two steps, priming, and activation, to exert its biological effects (S. Paik et al. Cell Mol Immunol 2021; 18:1141). The priming step is provided by inflammatory stimuli that involve Toll-like receptors (TLRs), which induce NFKB-mediated NLRP3 and pro-IL-1β expression and post-translational modifications of NLRP3. The activation step is triggered by exposure to damage-associated molecular patterns (DAMPs) and pathogen-associated molecular patterns (PAMPs) or other stimuli that promote NLRP3 inflammasome assembly and the final release of IL-1β and IL-1 (K V. Swanson et al. Nat Rev Immunol. 2019; 19:477).

The NLRP3 inflammasome has been associated with several inflammatory-based pathologies, including neurodegenerative and metabolic diseases, atherosclerosis, and cancer (K V. Swanson et al. Nat Rev Immunol. 2019; 19:477). The role of the NLRP3 inflammasome in the regulation of cancer has attracted increased attention in recent years, and research has identified a complex scenario in which NLRP3 acts as a double-edged sword. However, the clinical relevance of the NLRP3 inflammasome in the different phases of tumorigenesis may lead to a potential strategy for the development of novel anticancer therapies. (S. Missiroli et al. Cancers (Basel) 2021; 13:2297).

These findings prompted the development of potent and selective NLRP3 inhibitors that are extensively used as pharmacological tools to elucidate possible clinical applications of NLRP3 targeting strategies (M. Su et al. Curr Med Chem 2021; 28:569; X. Zhang et al. Eur J Med Chem 2020; 185:111822; Adam G Schwaid et al. J Med Chem 2021; 64:101-122). Among these, MCC950 N-((1,2,3,5,6,7-hexahydro-s-indacen-4-yl)carbamoyl)-4-(2-hydroxypropan-2-yl)furan-2-sulfonamide is the most studied NLRP3 inhibitor (RC Coll et al. Nat Med 2015; 21:248).

It has been demonstrated that MCC950 blocks canonical and noncanonical NLRP3 activation at nanomolar concentrations in vitro with high selectivity over AIM2, NLRC4, or NLRP1 inflammasomes. In preclinical studies, the compound exhibited efficacy in different in vivo models with good oral bioavailability and reached a phase II clinical trial for the potential treatment of rheumatoid arthritis. However, the study was discontinued since the molecule seemed to induce an elevation of serum liver enzyme levels for reasons that are still unclear (MSJ Mangan et al. Nat Rev Drug Discov 2018; 17:588). In cancer treatment, MCC950 was shown to suppress cell proliferation in chronic myeloid leukemia (S. Hamarsheh et al. Nat Commun 2020; 11:1659) and pancreatic adenocarcinoma (A. C. K. Yaw et al. J Cancer Res Clin Oncol 2020; 146:2219) and to delay tumor growth in a head and neck squamous cell carcinoma mouse model (L. Chen et al. Cell Mol Life Sci 2018; 75:2045).

The current treatment of immunopathologies NLRP3-related is based on the inhibition of cytokine IL-1β produced by the inflammasome. Three biological drugs were approved by U.S. Food and Drug Administration for the treatment of some inflammatory diseases: canakinumab, a monoclonal antibody neutralizing IL-1β; anakinra, an antagonist of IL-1 receptor; and rilonacept, a receptor “bait” that binds IL-1β e IL-1a. Two other biological drugs, GSK1070806, an antibody to IL-18, and MABp1, an antibody to IL-1α, are in the early stages of development (K V. Swanson et al. Nat Rev Immunol. 2019; 19:477).

Despite the therapeutic benefits of such biological drugs, they have demonstrated efficacy limits for some clinical indications and are typically characterized by high production costs.

Direct targeting of NLRP3 by low molecular weight molecules has the advantages of being a more selective, cost-effective, and less invasive approach to cytokine blocking. The activation of inflammasomes is in fact fundamental for the immune control of numerous pathogens, so the total loss of IL-1β can have harmful effects on the immune defense. Many of the new therapies that advance in clinical trials are specific for NLRP3 activation and do not affect the function of other inflammasomes. With the increase in the number of individuals suffering from inflammatory conditions, due to the lifestyle of Western countries and the aging of the population, there is likely to be a greater need for specific therapies for NLRP3. It is therefore felt the need to identify new compounds capable of selectively inhibiting NLRP3 that can be developed as an innovative therapeutic approach in oncology and, more generally, for the treatment of immunopathologies resulting from the overactivation of NLRP3 inflammasome resulting in overproduction of IL-1β.

### SUMMARY OF THE INVENTION

The inventors designed and identified new low molecular weight molecules that are able to selectively inhibit the activation of NLRP3 inflammasome by reducing the related production of IL-1β and IL-18, both in living mammalian cells and in an animal model in vivo.

The present invention hence relates to an hexahydro-s-indacene compound of Formula (I) or its pharmaceutically acceptable salt:

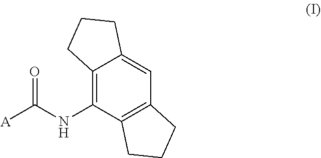

wherein A is selected from the group consisting of:

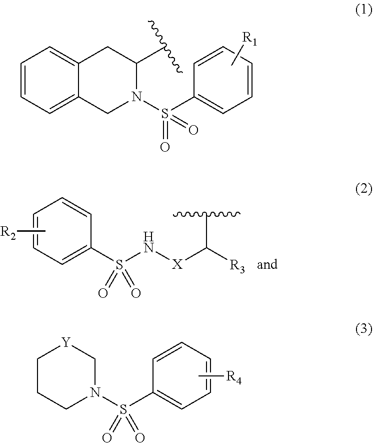

where
R₁ is a substituent selected from H, halogen, CF₃, (C₁-C₃)alkyl, (C₁-C₃)alkoxy, NH₂, NO₂, CN, COOH, a heterocyclic substituent selected from the group consisting of piperidine, morpholine and optionally substituted piperazine, —NHCH₂Ph, —N((C₁-C₂)alkyl)₂, —NH((C₂-C₄)alkyl)-NH₂, —COO((C₁-C₂)alkyl and —NHEt;
X is —(CH₂)ₙ— and n is equal to 0 o 1;
R₂ is a substituent selected from NH₂ and NO₂;
R₃ is a substituent selected from the group consisting of H, alkyl(C₁-C₄) alkyl(C₁-C₄)NH₂, phenyl, benzyl and hydroxybenzyl;
Y is —(CH₂)ₙ— and n is equal to 0 or 1; and
R₄ is a substituent selected from NH₂ and NO₂.

In a first preferred embodiment A is A1 and the invention relates to a tetrahydroisoquinoline compound of formula (II) or its pharmaceutically acceptable salt:

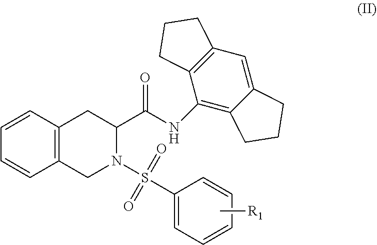

wherein
R₁ is a substituent selected from H, halogen, CF₃, (C₁-C₃)alkyl, (C₁-C₃)alkoxy, NH₂, NO₂, CN, COOH, a heterocyclic substituent selected from the group consisting of piperidine, morpholine and optionally substituted piperazine, —NHCH₂Ph, —N((C₁-C₂)alkyl)₂, —NH((C₂-C₄)alkyl)-NH₂, COO((C₁-C₂)alkyl and —NHEt.

In a second preferred embodiment A is A2 and the invention relates to a sulfonamide compound of formula (III) or its pharmaceutically acceptable salt:

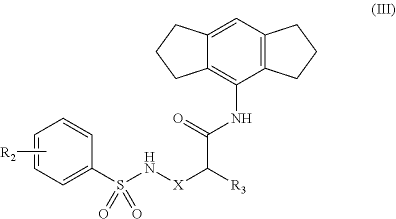

wherein
X is —(CH₂)ₙ— and n is equal to 0 o 1;
R₁ is a substituent selected from NH₂ and NO₂;
R₂ is a substituent selected from the group consisting of H, alkyl(C₁-C₄) alkyl(C₁-C₄)NH₂, phenyl, benzyl and hydroxybenzyl.

In a third preferred embodiment A is A3 and the invention relates to a pyrrolidine/piperidine compound of formula (IV) or its pharmaceutically acceptable salt:

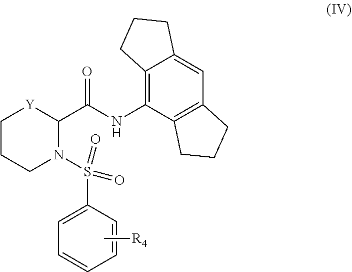

wherein
Y is —(CH₂)ₙ— and n is equal to 0 or 1; and
R₄ is a substituent selected from NH₂ and NO₂.

The compounds of the invention of formula (I), (II), (III) and (IV) were capable of treating pathologies related to NLRP3 hyperactivation and in particular of counteracting tumor growth in an animal model. The invention therefore concerns a therapeutic approach which comprises the use of new compounds useful for the clinical management of pathologies such as for example cancer, metabolic disorders, neurodegenerative diseases and autoimmune diseases.

In the present invention when referring to the compound of formula (I), (II), (III) and (IV), it is meant to include all possible tautomers and optical isomers, such as enantiomers and/or diastereoisomers or mixtures thereof (as racemates or in various ratios).

In another embodiment, the invention relates to an hexahydro-s-indacene compound of formula (I) or its pharmaceutically acceptable salts for use as a medicament.

In a further embodiment, the invention relates to an hexahydro-s-indacene compound of formula (I) or its pharmaceutically acceptable salts for use as a selective inhibitor of the NLRP3 inflammasome, preferably in the treatment of pathologies related to hyperactivation of NLRP3 and hyperproduction of interleukin 1p and interleukin 18.

The invention thus provided a compound of formula (I) as a modulator of NLRP3 in mammalian cells and tissues.

In the present invention “halogen” means fluorine, chlorine, bromine and iodine.

The invention also includes pharmaceutical compositions which contain one or more compounds of the invention of formula (I), (II), (III) and (IV) as active principle together with pharmaceutically acceptable excipients and possibly additives and stabilizers used in medicaments.

For a better understanding of the present invention, it is also described with reference to the attached figures.

### DESCRIPTION OF THE FIGURES

FIG. 1 shows the synthetic scheme for the preparation of compounds of formula (II) according to examples 1-6 (reagents and conditions indicated in figure: i. 4-Nitrobenzenesulfonyl chloride, NaHCO₃, H₂O, r.t., 16 h; ii. Hexahydro-s-indacen-4-amine, HATU, DIPEA, DMF, 0° C. to r.t., 2-5 h; iii. H₂ Pd/C, CH₃COOH, EtOAc, r.t, 16 h; iv. substituted benzenesulfonyl chloride, K₂CO₃, H₂O, THF, r.t., 2 h; v. various primary or secondary amines, DMSO, 120° C., 4-16 h; vi. 9 d, LiOH, H₂O, MeOH, r.t. 2 h).

FIG. 2, parts A, B, C, D and E, reports the results of examples 7, 8 e 9 specifically FIG. 2A reports cellular viability of THP-1 cells measured through RealTime-Glo MT Cell Viability assay. Cells have been treated with compound 4 at 1 μM of concentration and monitored for 48 hours; FIG. 2B reports IL-1 and IL-18 production from BMDMs and THP-1 cells stimulated with lipopolysaccharide (LPS) (1 μg/mL for 2 hours) treated with the compound 4 (1-1,000 nM) for 30 minutes and finally stimulated with ATP (5 mM for 1 hour) and measured by ELISA. Cytokine level is normalized to that of DMSO-treated control cells. Nonlinear regression analysis was performed; FIG. 2C reports the analysis, by western blot, of NLRP3 inflammasome components in THP-1 cells, before and after activation with LPS and ATP and compound 4. The expression of NLRP3 caspase-1, pro-IL-1 and ASC proteins has been analyzed on total cell lysate) and normalized on GAPDH (FIG. 2D); The expression of caspase-1 and IL-1β released has been analyzed on Media and normalized on ponceau (Ponceau S.) (FIG. 2C).

FIGS. 2E and 2F reports IL-1 production in supernatant from LPS-primed BMDMs treated with compound 4 and transfected with flagellin of S. typhimurium (E) or with poly(dA:dT) (F).

FIGS. 2G and 2H reports the results for NLRP-3-stimulated secretion of IL18.

FIG. 3, parts A and B, reports the results of example 10. Specifically, the figure reports IL-1 production from blood (part A) and peritoneal exudate (part B) in C57BL/6 mice pretreated with compounds or vehicle for 30 minutes and then treated with LPS (1 mg/kg) via intraperitoneal injection for 4 hours and measured by test ELISA. Data are presented as mean±SEM from three independent experiments. ** p<0.01, *** p<0.001, **** p<0.0001.

FIG. 4, parts A, B, C, D, and E reports the results of example 11 where C57BL/6 mice (5 for each condition) were subcutaneously inoculated with B16-F10cytLUC melanoma cells (1×10⁶). Specifically, FIG. 4A reports tumor growth kinetics for the indicated time points; FIG. 4B reports ex vivo quantification of tumor volumes assessed by a caliper 14 days post-injection; FIG. 4C reports representative excised tumors imaged 14 days post-injection; FIG. 4D reports representative pictures of cytLUC luminescence emission; FIG. 4E reports representative western blot showing the amount of NLRP3, caspase-1 and ASC proteins levels in tumors from B16-F10cytLUC melanoma cells inoculated in C57BL/6 mice and relative quantification. Error bars indicate SEM ** p<0.01, **** p<0.0001.

FIG. 4, parts F e G reports the results of examples 12 and 13 where the effect of compounds on B16-F10 tumoral cells growth is assessed. Specifically, FIG. 4F reports growth curve of B16-F10 cells after treatment with compounds; FIG. 4G reports the effects of the compounds on the tumoral microenvironment through the coculture model of peritoneal macrophages and B16-F10 cells. Error bars indicate SEM **** p<0.0001.

FIG. 5 shows the synthetic scheme for the preparation of compounds of formula (III) according to Examples 14, 15 and 16 (reagents and conditions indicated in figure: i. 4-Nitrobenzenesulfonyl chloride, NaHCO₃, H₂O, r.t., 16 h; ii. Hexahydro-s-indacen-4-amine, HATU, DIPEA, DMF, 0° C. to r.t., 2-5 h; iii. H₂ Pd/C, CH₃COOH, EtOAc, r.t, 16 h; iv. 4N HCl in dioxane, 2 h).

FIG. 6, parts A, B, C, D and E reports the results of the examples 17, 18 and 19. Specifically FIG. 6A reports cellular viability of THP-1 cells measured through RealTime-Glo MT Cell Viability assay. Cells have been treated with the selected compounds (6c and 10) at 1 μM of concentration and monitored for 48 hours; the FIG. 6B reports IL-1β production from BMDMs and THP-1 cells stimulated with lipopolysaccharide (LPS) (1 μg/mL for 2 hours) treated with the compounds (1-1,000 nM) for 30 minutes and finally stimulated with ATP (5 mM for 1 hour) and measured by ELISA. Cytokine level was normalized to that of DMSO-treated control cells. Nonlinear regression analysis was performed; FIG. 6C reports the analysis, by western blot, of NLRP3 inflammasome components in THP-1 cells, before and after activation with LPS and ATP and selected compounds. The expression of NLRP3, caspase-1, pro-IL-1β and ASC proteins has been analyzed on total cell lysate and normalized on GAPDH (FIG. 6D); The expression of caspase-1 and IL-1β released has been analyzed on Media and normalized on ponceau (Ponceau S.) (FIG. 6C).

FIGS. 6E and 6F reports IL-1 production in supernatant from LPS-primed BMDMs treated with compounds and transfected with flagellin of S. typhimurium (E) or with poly(dA:dT) (F).

FIGS. 6G and 6H report the results for NLRP3-stimulated secretion of IL-18 in BMDM and THP-1 cells, respectively. Data are presented as mean±SEM from three independent experiments. * p<0.05.

FIG. 7, parts A and B, reports the results of example 20. Specifically, the figure reports IL-1β production from blood (part A) and peritoneal surnatant (part B) in C57BL/6 mice pre-treated with compounds 6c and 10 or vehicle for 30 minutes and then treated with LPS (1 mg/kg) via intraperitoneal injection for 4 hours and measured by test ELISA. Data are presented as mean±SEM from three independent experiments. ** p<0.01, *** p<0.001, **** p<0.0001.

FIG. 8, parts A, B, C, D, E reports the results of example 21 where C57BL/6 mice (5 for each condition) were subcutaneously inoculated with B16-F10cytLUC melanoma cells (1×10⁶). Specifically, FIG. 8A reports tumor growth kinetics for the indicated time points; FIG. 8B reports ex vivo quantification of tumor volumes assessed by a caliper 14 days post-injection; FIG. 8C reports representative excised tumors imaged 14 days post-injection; FIG. 8D reports representative pictures of cytLUC luminescence emission; FIG. 8E reports representative western blot showing the amount of NLRP3, caspase-1, ASC and pro-IL1p proteins levels in tumors from B16-F10cytLUC melanoma cells inoculated in C57BL/6 mice and relative quantification. Error bars indicate SEM. ** p<0.001, **** p<0.0001.

FIG. 8, parts F e G reports the results of examples 22 and 23 where the effect of compounds on B16-F10 tumoral cells growth is assessed. Specifically, FIG. 8F reports growth curve of B16-F10 cells after treatment with compounds; FIG. 8G reports the effects of the compounds on the tumoral microenvironment through the coculture model of peritoneal macrophages and B16-F10 cells. Error bars indicate SEM **** p<0.0001.

FIG. 9 shows the synthetic scheme for the preparation of compounds of formula (IV) according to Examples 24, 25 and 26.

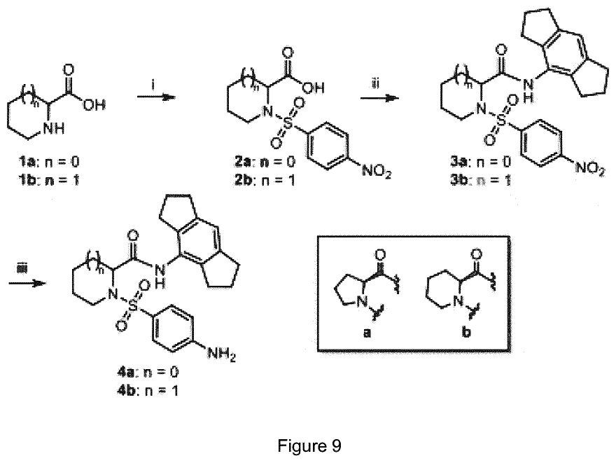

The compounds of formula (IV) were obtained starting from the a amino acids proline (1a) or pipecolic acid (1b) which were reacted with 4-nitrobenzenesulfonyl chloride in the presence of NaHCO₃ to obtain the intermediates 2a-b (FIG. 9). Subsequent amide coupling with hexahydro-s-indacen-4-amine in the presence of HATU and DIPEA gave compounds 3a-b. These were finally reduced by catalytic hydrogenation to the corresponding aniline derivatives 4a-b. (reagents and conditions indicated in figure: i. 4-Nitrobenzenesulfonyl chloride, NaHCO₃, H₂O, r.t., 16 h; ii. Hexahydro-s-indacen-4-amine, HATU, DIPEA, DMF, 0° C. to r.t., 2-5 h; iii. H₂ Pd/C, CH₃COOH, EtOAc, r.t, 16 h).

### DETAILED DESCRIPTION OF THE INVENTION

The present invention hence relates to an hexahydro-s-indacene compound of Formula (I) or its pharmaceutically acceptable salt:

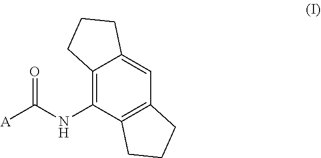

wherein A is selected from the group consisting of:

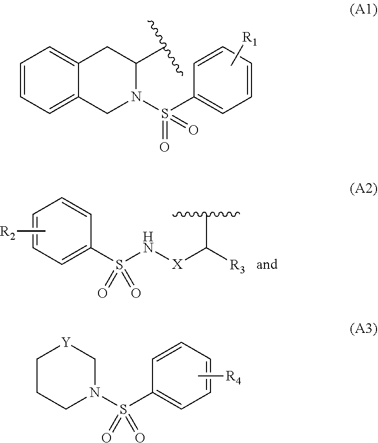

where
R₁ is a substituent selected from H, halogen, CF₃, (C₁-C₃)alkyl, (C₁-C₃)alkoxy, NH₂, NO₂, CN, COOH, a heterocyclic substituent selected from the group consisting of piperidine, morpholine and optionally substituted piperazine, —NHCH₂Ph, —N((C₁-C₂)alkyl)₂, —NH((C₂-C₄)alkyl)-NH₂, COO((C₁-C₂)alkyl and —NHEt;
X is —(CH₂)ₙ— and n is equal to 0 o 1;
R₂ is a substituent selected from NH₂ and NO₂;
R₃ is a substituent selected from the group consisting of H, alkyl(C₁-C₄) alkyl(C₁-C₄)NH₂, phenyl, benzyl and hydroxybenzyl;
Y is —(CH₂)ₙ— and n is equal to 0 or 1; and
R₄ is a substituent selected from NH₂ and NO₂.

In the present invention, when the following terms are used:
“compound(s) of the invention” means a compound of Formula (I), as defined above, in any form, i.e. any saline or non-salt form and any physical form including non-solid forms and any solid, crystalline, amorphous, polymorphic, solvated form including hydrates, e.g. mono, di hemihydrates and various mixtures of these forms;
“(C₁-C₄)alkyl” means a linear or branched hydrocarbon group containing 1 to 4 carbon atoms;
“(C₁-C₃)alkyl-NH₂” means a linear or branched hydrocarbon group containing 1 to 3 carbon atoms bonded to an amino group.—
“(C₁-C₃)alkyl” means a linear or branched hydrocarbon group containing 1 to 3 carbon atoms;
“(C₁-C₂)alkyl” means a linear hydrocarbon group containing 1 to 2 carbon atoms;
“(C₂-C₄)alkyl” means a linear or branched hydrocarbon group containing from two to four carbon atoms;
“(C₁-C₃)alcoxy” means a substituent of structure (alkyl chain) —O— having from 1 to 3 carbon atoms;
“halogen” means fluorine, chlorine, bromine and iodine;
“optionally substituted” means that the structure may not be substituted or substituted with a particular substitute;
“a heterocyclic substituent selected from the group consisting of piperidine, morpholine and optionally substituted piperazine” means a heterocyclic substituent selected from piperidine, morpholine and piperazine, the latter being optionally substituted on the nitrogen atom by a substituent, and
“a compound of formula (I), (II), (III), (IV)” includes all possible tautomers and optical isomers, such as enantiomers and/or diastereoisomers or mixtures thereof (as racemates or in various ratios) and possible pharmaceutically acceptable salts.

In a first preferred embodiment of the invention, A is A1 and the compound of Formula (I) is a tetrahydroisoquinoline compound of formula (II) or its pharmaceutically acceptable salt:

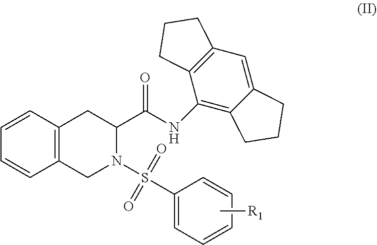

wherein
R₁ is a substituent selected from H, halogen, CF₃, (C₁-C₃)alkyl, (C₁-C₃)alkoxy, NH₂, NO₂, CN, COOH, a heterocyclic substituent selected from the group consisting of piperidine, morpholine and optionally substituted piperazine, —NHCH₂Ph, —N((C₁-C₂)alkyl)₂, —NH((C₂-C₄)alkyl)-NH₂, COO((C₁-C₂)alkyl and —NHEt.

According to the invention, R₁ is a substituent selected from H, halogen, CF₃, (C₁-C₃)alkyl, (C₁-C₃)alkoxy, NH₂, NO₂, CN, COOH, a heterocyclic substituent selected from the group consisting of piperidine, morpholine and optionally substituted piperazine, —NHCH₂Ph, —N((C₁-C₂)alkyl)₂, —NH((C₂-C₄)alkyl)-NH₂; COO(C₁-C₂)alkyl and —NHEt.

Preferably R₁ is NH₂ or NO₂.

When R₁ is (C₁-C₃)alkyl, it can be methyl, ethyl, propyl or isopropyl.

When R₁ is halogen, it is fluorine, chlorine, bromine or iodine, preferably fluorine.

When R₁ is (C₁-C₃)alkoxy, it is preferably methoxy, ethoxy, propoxy or isopropoxy.

When R₁ is a heterocyclic substituent selected from the group consisting of piperidine, morpholine and optionally substituted piperazine, it is preferably piperidine, morpholine or piperazine, the latter being optionally substituted on the nitrogen atom with, preferably, methyl or tert-butoxycarbonyl. Such heterocyclic substituent is preferably selected from the group consisting of

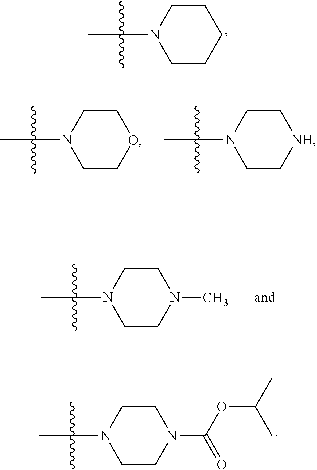

When R₁ is N((C₁-C₂)alkyl)₂, it is preferably N(CH₂CH₃)₂ or —NHCH₂CH₂NH₂.

When R₁ is NH((C₂-C₄)alkyl)-NH₂ it is preferably NHCH₂CH₂NH₂ or NHCH₂CH₂CH₂CH₂NH₂.

When R₁ is COO((C₁-C₂)alkyl, it is COOMe or COOEt

In an advantageous and preferred embodiment, R₁ is NH₂ or NO₂.

Advantageously, the invention relates to a tetrahydroisoquinoline compound of formula (II) selected from the group consisting of:
(S)—N-(1,2,3,5,6,7-hexahydro-s-indacen-4-yl)-2-((4-nitrophenyl)sulfonyl)-1,2,3,4-tetrahydroisoquinoline-3-carboxamide (3 of Formula (II))

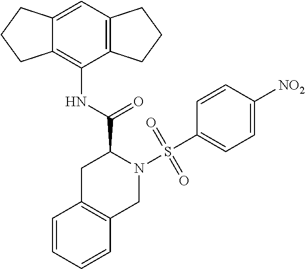 [SMILES: O=C(Nc1c2c(cc3c1CCC3)CCC2)[C@@H]1Cc2ccccc2CN1S(=O)(=O)c1ccc([N+](=O)[O-])cc1]

(S)-2-((4-aminophenyl)sulfonyl)-N-(1,2,3,5,6,7-hexahydro-s-indacen-4-yl)-1,2,3,4-tetrahydroisoquinoline-3-carboxamide (4 of Formula (II))

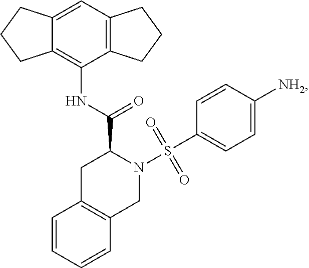 [SMILES: Nc1ccc(S(=O)(=O)N2Cc3ccccc3C[C@H]2C(=O)Nc2c3c(cc4c2CCC4)CCC3)cc1]

(S)-2-((4-fluorophenyl)sulfonyl)-N-(1,2,3,5,6,7-hexahydro-s-indacen-4-yl)-1,2,3,4-tetrahydroisoquinoline-3-carboxamide (6 of Formula (II))

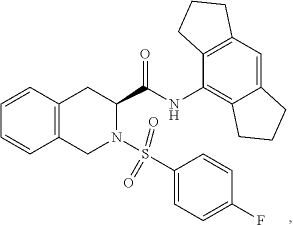 [SMILES: O=C(Nc1c2c(cc3c1CCC3)CCC2)[C@@H]1Cc2ccccc2CN1S(=O)(=O)c1ccc(F)cc1]

(S)-2-((4-(benzylamino)phenyl)sulfonyl)-N-(1,2,3,5,6,7-hexahydro-s-indacen-4-yl)-1,2,3,4-tetrahydroisoquinoline-3-carboxamide (7a of Formula (II))

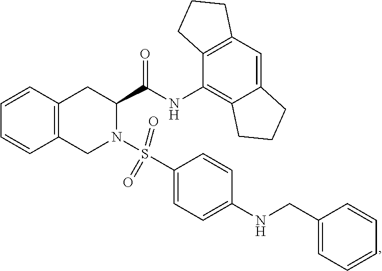 [SMILES: O=C(Nc1c2c(cc3c1CCC3)CCC2)[C@@H]1Cc2ccccc2CN1S(=O)(=O)c1ccc(NCc2ccccc2)cc1]

(S)-2-((4-(dimethylamino)phenyl)sulfonyl)-N-(1,2,3,5,6,7-hexahydro-s-indacen-4-yl)-1,2,3,4-tetrahydroisoquinoline-3-carboxamide (7b of Formula (II))

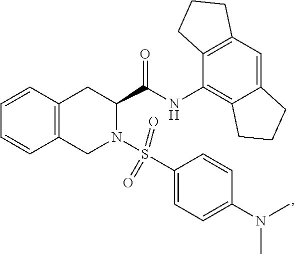 [SMILES: CN(C)c1ccc(S(=O)(=O)N2Cc3ccccc3C[C@H]2C(=O)Nc2c3c(cc4c2CCC4)CCC3)cc1]

(S)—N-(1,2,3,5,6,7-hexahydro-s-indacen-4-yl)-2-((4-(piperidin-1-yl)phenyl) sulfonyl)-1,2,3,4-tetrahydroisoquinoline-3-carboxamide (7c of Formula (II))

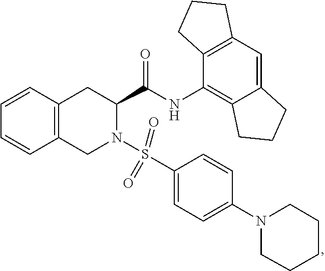 [SMILES: O=C(Nc1c2c(cc3c1CCC3)CCC2)[C@@H]1Cc2ccccc2CN1S(=O)(=O)c1ccc(N2CCCCC2)cc1]

5 (S)—N-(1,2,3,5,6,7-hexahydro-s-indacen-4-yl)-2-((4-morpholinophenyl)sulfonyl)-1,2,3,4-tetrahydroisoquinoline-3-carboxamide (7 d of Formula (II))

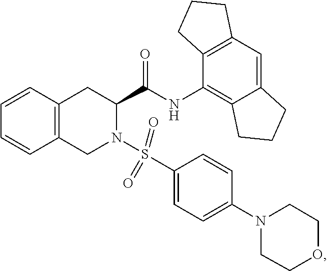 [SMILES: O=C(Nc1c2c(cc3c1CCC3)CCC2)[C@@H]1Cc2ccccc2CN1S(=O)(=O)c1ccc(N2CCOCC2)cc1]

(S)-2-((4-(diethylamino)phenyl)sulfonyl)-N-(1,2,3,5,6,7-hexahydro-s-indacen-4-yl)-1,2,3,4-tetrahydroisoquinoline-3-carboxamide (7e of Formula (II))

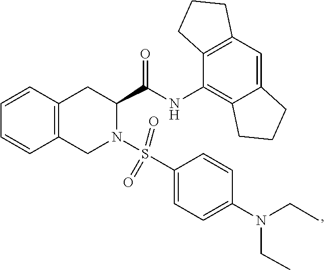 [SMILES: CCN(CC)c1ccc(S(=O)(=O)N2Cc3ccccc3C[C@H]2C(=O)Nc2c3c(cc4c2CCC4)CCC3)cc1]

(S)—N-(1,2,3,5,6,7-hexahydro-s-indacen-4-yl)-2-((4-(piperazin-1-yl)phenyl) sulfonyl)-1,2,3,4-tetrahydroisoquinoline-3-carboxamide (7f of Formula (II))

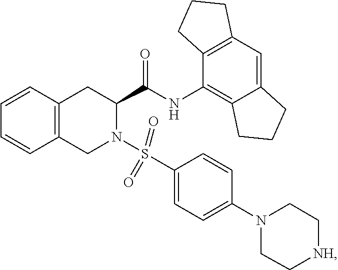 [SMILES: O=C(Nc1c2c(cc3c1CCC3)CCC2)[C@@H]1Cc2ccccc2CN1S(=O)(=O)c1ccc(N2CCNCC2)cc1]

(S)—N-(1,2,3,5,6,7-hexahydro-s-indacen-4-yl)-2-((4-(4-methylpiperazin-1-yl) phenyl)sulfonyl)-1,2,3,4-tetrahydroisoquinoline-3-carboxamide (7g of Formula (II))

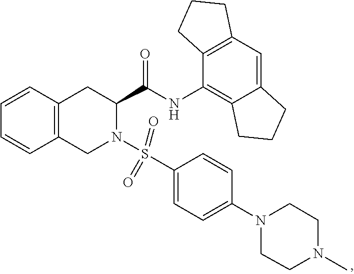 [SMILES: CN1CCN(c2ccc(S(=O)(=O)N3Cc4ccccc4C[C@H]3C(=O)Nc3c4c(cc5c3CCC5)CCC4)cc2)CC1]

tert-butyl(S)-4-(4-((3-((1,2,3,5,6,7-hexahydro-s-indacen-4-yl)carbamoyl)-3,4-dihydroisoquinolin-2(1H)-yl)sulfonyl)phenyl)piperazine-1-carboxylate (7h of Formula (II))

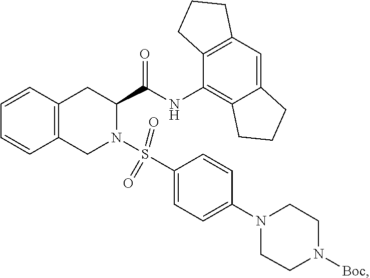 [SMILES: CC(C)(C)OC(=O)N1CCN(c2ccc(S(=O)(=O)N3Cc4ccccc4C[C@H]3C(=O)Nc3c4c(cc5c3CCC5)CCC4)cc2)CC1]

(S)-2-((4-(ethylamino)phenyl)sulfonyl)-N-(1,2,3,5,6,7-hexahydro-s-indacen-4-yl)-1,2,3,4-tetrahydroisoquinoline-3-carboxamide (7i of Formula (II))

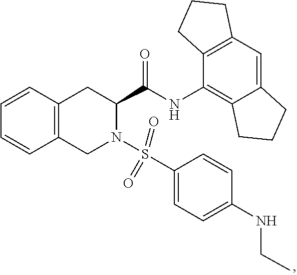 [SMILES: CCNc1ccc(S(=O)(=O)N2Cc3ccccc3C[C@H]2C(=O)Nc2c3c(cc4c2CCC4)CCC3)cc1]

(S)-2-((4-((2-aminoethyl)amino)phenyl)sulfonyl)-N-(1,2,3,5,6,7-hexahydro-s-indacen-4-yl)-1,2,3,4-tetrahydroisoquinoline-3-carboxamide (7j of Formula (II))

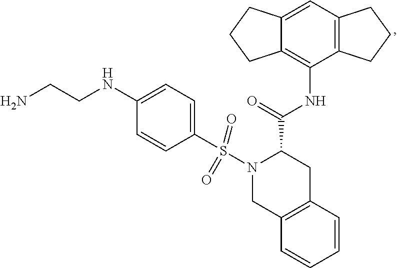 [SMILES: NCCNc1ccc(S(=O)(=O)N2Cc3ccccc3C[C@H]2C(=O)Nc2c3c(cc4c2CCC4)CCC3)cc1]

(S)-2-((4-((4-aminobutyl)amino)phenyl)sulfonyl)-N-(1,2,3,5,6,7-hexahydro-s-indacen-4-yl)-1,2,3,4-tetrahydroisoquinoline-3-carboxamide (7k of Formula (II))

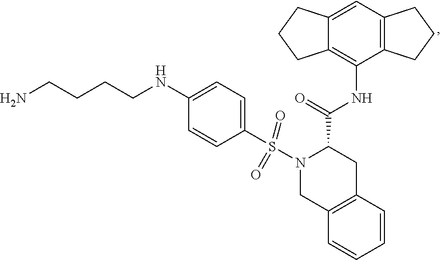 [SMILES: NCCCCNc1ccc(S(=O)(=O)N2Cc3ccccc3C[C@H]2C(=O)Nc2c3c(cc4c2CCC4)CCC3)cc1]

(S)—N-(1,2,3,5,6,7-hexahydro-s-indacen-4-yl)-2-((4-(trifluoromethyl)phenyl)sulfonyl)-1,2,3,4-tetrahydroisoquinoline-3-carboxamide (9a of Formula (II))

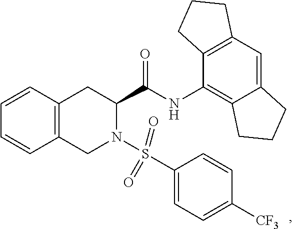 [SMILES: O=C(Nc1c2c(cc3c1CCC3)CCC2)[C@@H]1Cc2ccccc2CN1S(=O)(=O)c1ccc(C(F)(F)F)cc1]

(S)—N-(1,2,3,5,6,7-hexahydro-s-indacen-4-yl)-2-((4-methoxyphenyl)sulfonyl)-1,2,3,4-tetrahydroisoquinoline-3-carboxamide (9b of Formula (II))

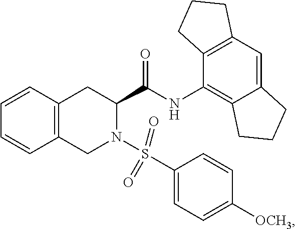 [SMILES: COc1ccc(S(=O)(=O)N2Cc3ccccc3C[C@H]2C(=O)Nc2c3c(cc4c2CCC4)CCC3)cc1]

(S)-2-((4-cyanophenyl)sulfonyl)-N-(1,2,3,5,6,7-hexahydro-s-indacen-4-yl)-1,2,3,4-tetrahydroisoquinoline-3-carboxamide (9c of Formula (II))

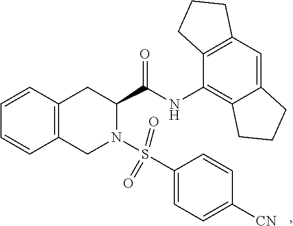 [SMILES: N#Cc1ccc(S(=O)(=O)N2Cc3ccccc3C[C@H]2C(=O)Nc2c3c(cc4c2CCC4)CCC3)cc1]

(S)-Methyl 4-((3-((1,2,3,5,6,7-hexahydro-s-indacen-4-yl)carbamoyl)-3,4-dihydroisoquinolin-2(1H)-yl)sulfonyl)benzoate (9 d of Formula (II))

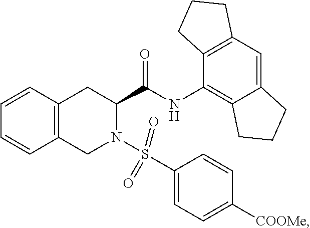 [SMILES: COC(=O)c1ccc(S(=O)(=O)N2Cc3ccccc3C[C@H]2C(=O)Nc2c3c(cc4c2CCC4)CCC3)cc1]

(S)-4-((3-((1,2,3,5,6,7-hexahydro-s-indacen-4-yl)carbamoyl)-3,4-dihydroisoquinolin-2(1H)-yl)sulfonyl)benzoic acid (10 of Formula (II))

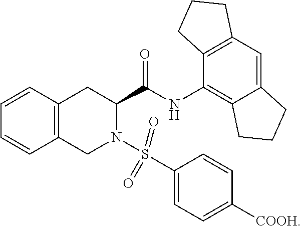 [SMILES: O=C(O)c1ccc(S(=O)(=O)N2Cc3ccccc3C[C@H]2C(=O)Nc2c3c(cc4c2CCC4)CCC3)cc1]

More preferably and advantageously, the tetrahydroisoquinoline compound of formula (II) is selected from the group consisting of:
(S)—N-(1,2,3,5,6,7-hexahydro-s-indacen-4-yl)-2-((4-nitrophenyl)sulfonyl)-1,2,3,4-tetrahydroisoquinoline-3-carboxamide (3 of Formula (II))

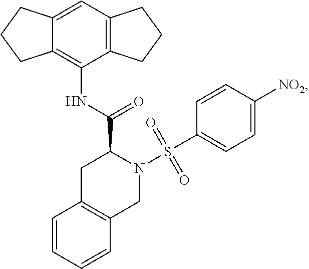 [SMILES: O=C(Nc1c2c(cc3c1CCC3)CCC2)[C@@H]1Cc2ccccc2CN1S(=O)(=O)c1ccc([N+](=O)[O-])cc1]

(S)-2-((4-aminophenyl)sulfonyl)-N-(1,2,3,5,6,7-hexahydro-s-indacen-4-yl)-1,2,3,4-tetrahydroisoquinoline-3-carboxamide (4 of Formula (II))

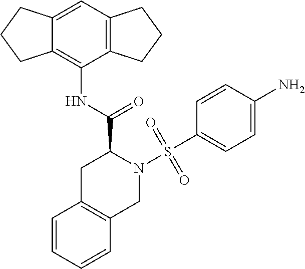 [SMILES: Nc1ccc(S(=O)(=O)N2Cc3ccccc3C[C@H]2C(=O)Nc2c3c(cc4c2CCC4)CCC3)cc1]

Without being bound to any theory, the inventors believe that the basic structure of the molecule of Formula (II) is active as a selective inhibitor of the NLRP3 inflammasome precisely in the definition of R₁.

In a second preferred embodiment, A is A2 and the invention relates to a sulfonamide compound of formula (III) or its pharmaceutically acceptable salt:

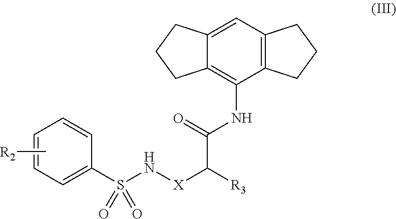

wherein
X is —(CH₂)ₙ— and n is equal to 0 o 1;
R₂ is a substituent selected from NH₂ and NO₂;
R₃ is a substituent selected from the group consisting of H, alkyl(C₁-C₄) alkyl(C₁-C₄)NH₂, phenyl, benzyl and hydroxybenzyl.

According to the invention X is —(CH₂)ₙ— and n is equal to 0 or 1. Preferably n equals 0.

R₂ is a substituent selected from NH₂ and NO₂, while R₃ is a substituent selected from the group consisting of H, alkyl(C₁-C₄), alkyl(C₁-C₄)NH₂, phenyl, benzyl and hydroxybenzyl;
When R₃ is alkyl(C₁-C₄), it is preferably CH₃, CH(CH₃)₂, CH₂CH(CH₃)₂.

When R₃ is alkyl(C₁-C₄)NH₂, it is preferably —(CH₂)₄NH₂.

More preferably R₃ is isopropyl, CH₂CH(CH₃)₂, benzyl o hydroxy-benzyl, even more preferably isopropyl o p-hydroxy-benzyl.

Advantageously, the invention relates to a sulfonamide compound of formula (III) selected from the group consisting of:
N-(1,2,3,5,6,7-hexahydro-s-indacen-4-yl)-2-((4-nitrophenyl)sulfonamido)acetamide (6a of Formula (III))

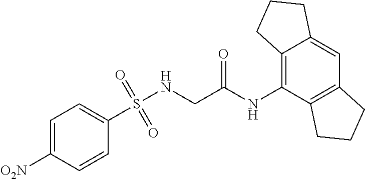 [SMILES: O=C(CNS(=O)(=O)c1ccc([N+](=O)[O-])cc1)Nc1c2c(cc3c1CCC3)CCC2]

(S)—N-(1,2,3,5,6,7-hexahydro-s-indacen-4-yl)-2-((4-nitrophenyl)sulfonamido)propanamide (6b of Formula (III))

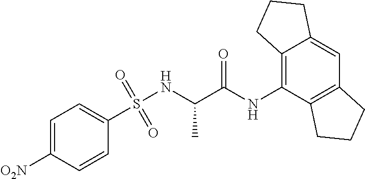 [SMILES: C[C@H](NS(=O)(=O)c1ccc([N+](=O)[O-])cc1)C(=O)Nc1c2c(cc3c1CCC3)CCC2]

(S)—N-(1,2,3,5,6,7-hexahydro-s-indacen-4-yl)-3-methyl-2-((4-nitrophenyl)sulfonamido)butanamide (6c of Formula (III))

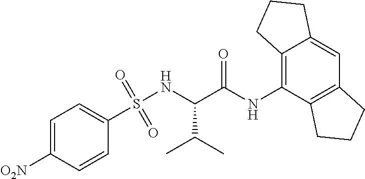 [SMILES: CC(C)[C@H](NS(=O)(=O)c1ccc([N+](=O)[O-])cc1)C(=O)Nc1c2c(cc3c1CCC3)CCC2]

(R)—N-(1,2,3,5,6,7-hexahydro-s-indacen-4-yl)-3-methyl-2-((4-nitrophenyl)sulfonamido)butanamide (6d of Formula (III))

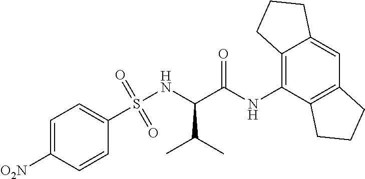 [SMILES: CC(C)[C@@H](NS(=O)(=O)c1ccc([N+](=O)[O-])cc1)C(=O)Nc1c2c(cc3c1CCC3)CCC2]

(S)—N-(1,2,3,5,6,7-hexahydro-s-indacen-4-yl)-4-methyl-2-((4-nitrophenyl)sulfonamido)pentanamide (6e of Formula (III))

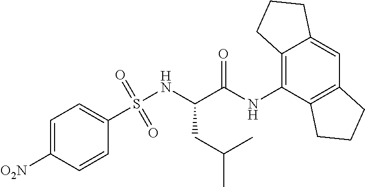 [SMILES: CC(C)C[C@H](NS(=O)(=O)c1ccc([N+](=O)[O-])cc1)C(=O)Nc1c2c(cc3c1CCC3)CCC2]

(S)—N-(1,2,3,5,6,7-hexahydro-s-indacen-4-yl)-2-((4-nitrophenyl)sulfonamido)-2-phenylacetamide (6f of Formula (III))

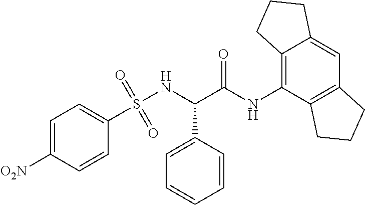 [SMILES: O=C(Nc1c2c(cc3c1CCC3)CCC2)[C@@H](NS(=O)(=O)c1ccc([N+](=O)[O-])cc1)c1ccccc1]

(S)—N-(1,2,3,5,6,7-hexahydro-s-indacen-4-yl)-2-((4-nitrophenyl)sulfonamido)-3-phenylpropanamide (6g of Formula (III))

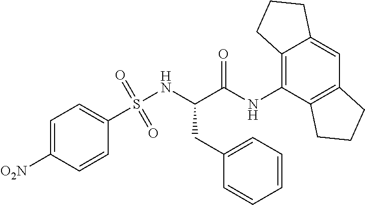 [SMILES: O=C(Nc1c2c(cc3c1CCC3)CCC2)[C@H](Cc1ccccc1)NS(=O)(=O)c1ccc([N+](=O)[O-])cc1]

(R)—N-(1,2,3,5,6,7-hexahydro-s-indacen-4-yl)-2-((4-nitrophenyl)sulfonamido)-3-phenylpropanamide (6h of Formula (III))

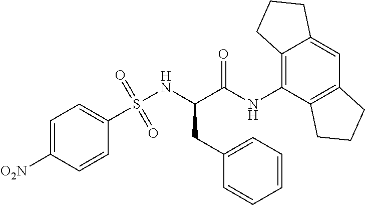 [SMILES: O=C(Nc1c2c(cc3c1CCC3)CCC2)[C@@H](Cc1ccccc1)NS(=O)(=O)c1ccc([N+](=O)[O-])cc1]

(S)—N-(1,2,3,5,6,7-hexahydro-s-indacen-4-yl)-3-(4-hydroxyphenyl)-2-((4-nitrophenyl)sulfonamido)propanamide (8 of Formula (III))

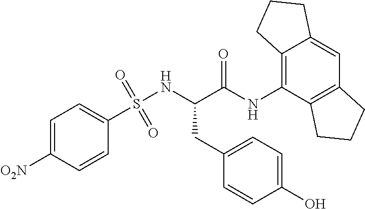 [SMILES: O=C(Nc1c2c(cc3c1CCC3)CCC2)[C@H](Cc1ccc(O)cc1)NS(=O)(=O)c1ccc([N+](=O)[O-])cc1]

(S)-6-amino-N-(1,2,3,5,6,7-hexahydro-s-indacen-4-yl)-2-((4-nitrophenyl)sulfonamido)hexanamide (9 of Formula (III))

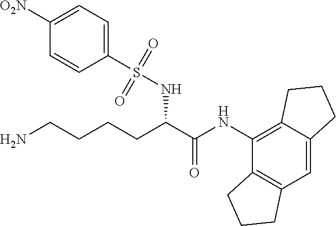 [SMILES: NCCCC[C@H](NS(=O)(=O)c1ccc([N+](=O)[O-])cc1)C(=O)Nc1c2c(cc3c1CCC3)CCC2]

N-(1,2,3,5,6,7-hexahydro-s-indacen-4-yl)-3-((4-nitrophenyl)sulfonamido)propanamide (6k of Formula (III))

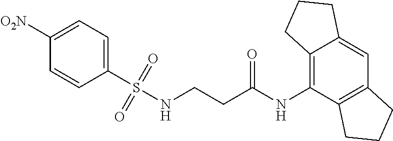 [SMILES: O=C(CCNS(=O)(=O)c1ccc([N+](=O)[O-])cc1)Nc1c2c(cc3c1CCC3)CCC2]

2-((4-aminophenyl)sulfonamido)-N-(1,2,3,5,6,7-hexahydro-s-indacen-4-yl)acetamide (7a of Formula (III))

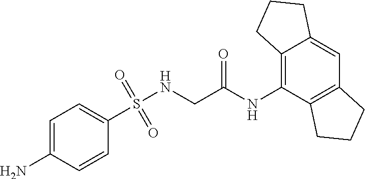 [SMILES: Nc1ccc(S(=O)(=O)NCC(=O)Nc2c3c(cc4c2CCC4)CCC3)cc1]

(S)-2-((4-aminophenyl)sulfonamido)-N-(1,2,3,5,6,7-hexahydro-s-indacen-4-yl)propanamide (7b of Formula (III))

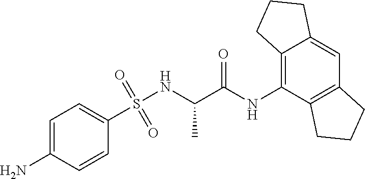 [SMILES: C[C@H](NS(=O)(=O)c1ccc(N)cc1)C(=O)Nc1c2c(cc3c1CCC3)CCC2]

(S)-2-((4-aminophenyl)sulfonamido)-N-(1,2,3,5,6,7-hexahydro-s-indacen-4-yl)-3-methylbutanamide (7c of Formula (III))

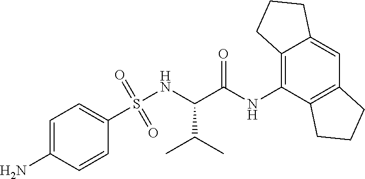 [SMILES: CC(C)[C@H](NS(=O)(=O)c1ccc(N)cc1)C(=O)Nc1c2c(cc3c1CCC3)CCC2]

(R)-2-((4-aminophenyl)sulfonamido)-N-(1,2,3,5,6,7-hexahydro-s-indacen-4-yl)-3-methylbutanamide (7 d of Formula (III))

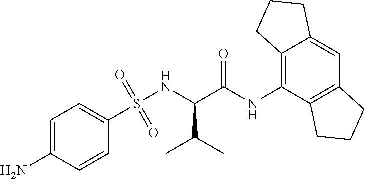 [SMILES: CC(C)[C@@H](NS(=O)(=O)c1ccc(N)cc1)C(=O)Nc1c2c(cc3c1CCC3)CCC2]

(S)-2-((4-aminophenyl)sulfonamido)-N-(1,2,3,5,6,7-hexahydro-s-indacen-4-yl)-4-methylpentanamide (7e of Formula (III))

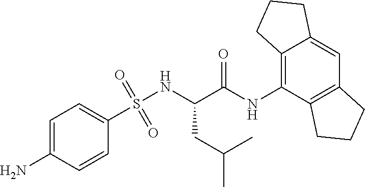 [SMILES: CC(C)C[C@H](NS(=O)(=O)c1ccc(N)cc1)C(=O)Nc1c2c(cc3c1CCC3)CCC2]

(S)-2-((4-aminophenyl)sulfonamido)-N-(1,2,3,5,6,7-hexahydro-s-indacen-4-yl)-2-phenylacetamide (7f of Formula (III))

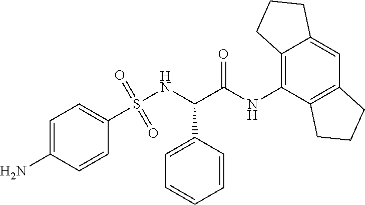 [SMILES: Nc1ccc(S(=O)(=O)N[C@H](C(=O)Nc2c3c(cc4c2CCC4)CCC3)c2ccccc2)cc1]

(S)-2-((4-aminophenyl)sulfonamido)-N-(1,2,3,5,6,7-hexahydro-s-indacen-4-yl)-3-phenylpropanamide (7g of Formula (III))

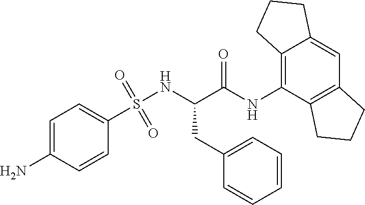 [SMILES: Nc1ccc(S(=O)(=O)N[C@@H](Cc2ccccc2)C(=O)Nc2c3c(cc4c2CCC4)CCC3)cc1]

(R)-2-((4-aminophenyl)sulfonamido)-N-(1,2,3,5,6,7-hexahydro-s-indacen-4-yl)-3-phenylpropanamide (7h of Formula (III))

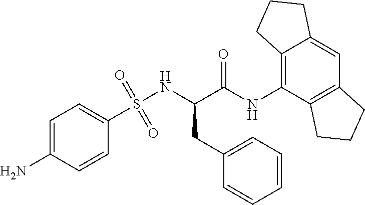 [SMILES: Nc1ccc(S(=O)(=O)N[C@H](Cc2ccccc2)C(=O)Nc2c3c(cc4c2CCC4)CCC3)cc1]

(S)-2-((4-aminophenyl)sulfonamido)-N-(1,2,3,5,6,7-hexahydro-s-indacen-4-yl)-3-(4-hydroxyphenyl)propanamide (10 of Formula (III))

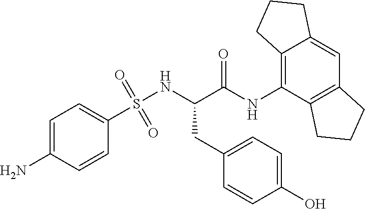 [SMILES: Nc1ccc(S(=O)(=O)N[C@@H](Cc2ccc(O)cc2)C(=O)Nc2c3c(cc4c2CCC4)CCC3)cc1]

(S)-6-amino-2-((4-aminophenyl)sulfonamido)-N-(1,2,3,5,6,7-hexahydro-s-indacen-4-yl)hexanamide (11 of Formula (III))

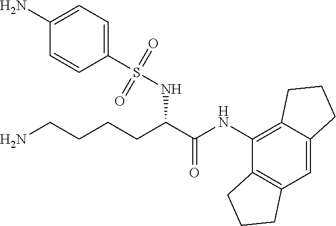 [SMILES: NCCCC[C@H](NS(=O)(=O)c1ccc(N)cc1)C(=O)Nc1c2c(cc3c1CCC3)CCC2]

3-((4-aminophenyl)sulfonamido)-N-(1,2,3,5,6,7-hexahydro-s-indacen-4-yl)propanamide (7k of Formula (III))

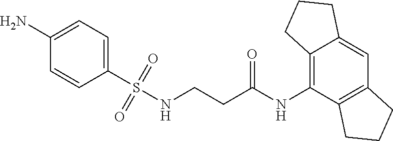 [SMILES: Nc1ccc(S(=O)(=O)NCCC(=O)Nc2c3c(cc4c2CCC4)CCC3)cc1]

Preferably, the compound of the invention with formula (III) is selected from the group consisting of:
(S)—N-(1,2,3,5,6,7-hexahydro-s-indacen-4-yl)-3-methyl-2-((4-nitrophenyl)sulfonamido)butanamide (6c of Formula (III))

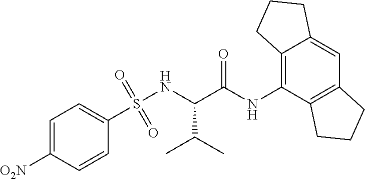 [SMILES: CC(C)[C@H](NS(=O)(=O)c1ccc([N+](=O)[O-])cc1)C(=O)Nc1c2c(cc3c1CCC3)CCC2]

(R)—N-(1,2,3,5,6,7-hexahydro-s-indacen-4-yl)-2-((4-nitrophenyl)sulfonamido)-3-phenylpropanamide (6h of Formula (III))

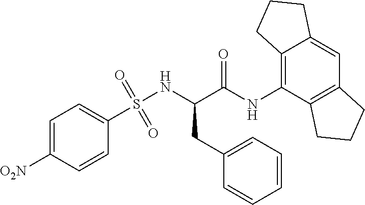 [SMILES: O=C(Nc1c2c(cc3c1CCC3)CCC2)[C@@H](Cc1ccccc1)NS(=O)(=O)c1ccc([N+](=O)[O-])cc1]

(S)-2-((4-aminophenyl)sulfonamido)-N-(1,2,3,5,6,7-hexahydro-s-indacen-4-yl)-3-methylbutanamide (7c of Formula (III))

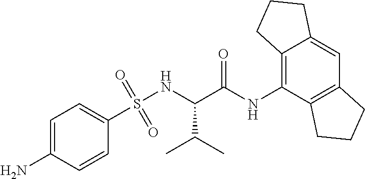 [SMILES: CC(C)[C@H](NS(=O)(=O)c1ccc(N)cc1)C(=O)Nc1c2c(cc3c1CCC3)CCC2]

(S)—N-(1,2,3,5,6,7-hexahydro-s-indacen-4-yl)-4-methyl-2-((4-nitrophenyl)sulfonamido)pentanamide (6e of Formula (III))

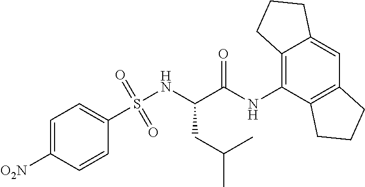 [SMILES: CC(C)C[C@H](NS(=O)(=O)c1ccc([N+](=O)[O-])cc1)C(=O)Nc1c2c(cc3c1CCC3)CCC2]

(S)-2-((4-aminophenyl)sulfonamido)-N-(1,2,3,5,6,7-hexahydro-s-indacen-4-yl)-3-(4-hydroxyphenyl)propanamide (10 of Formula (III))

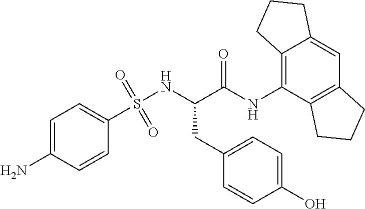 [SMILES: Nc1ccc(S(=O)(=O)N[C@@H](Cc2ccc(O)cc2)C(=O)Nc2c3c(cc4c2CCC4)CCC3)cc1]

More preferably and advantageously, the sulfonamide compound of formula (III) is selected from the group consisting of:
(S)—N-(1,2,3,5,6,7-hexahydro-s-indacen-4-yl)-3-methyl-2-((4-nitrophenyl)sulfonamido)butanamide (6c of Formula (III))

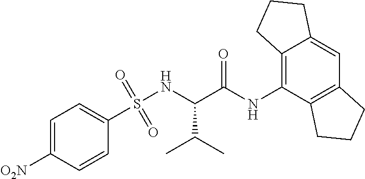 [SMILES: CC(C)[C@H](NS(=O)(=O)c1ccc([N+](=O)[O-])cc1)C(=O)Nc1c2c(cc3c1CCC3)CCC2]

(S)-2-((4-aminophenyl)sulfonamido)-N-(1,2,3,5,6,7-hexahydro-s-indacen-4-yl)-3-(4-hydroxyphenyl)propanamide (10 of Formula (III))

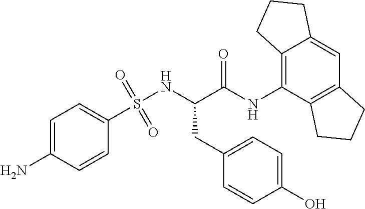 [SMILES: Nc1ccc(S(=O)(=O)N[C@@H](Cc2ccc(O)cc2)C(=O)Nc2c3c(cc4c2CCC4)CCC3)cc1]

In a third preferred embodiment, A is A3 and the invention relates to a pyrrolidine/piperidine compound of formula (IV) or its pharmaceutically acceptable salt:

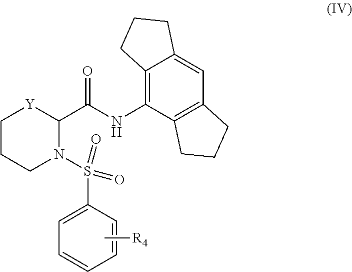

Y is —(CH₂)ₙ— and n is equal to 0 or 1; and
R₄ is a substituent selected from NH₂ and NO₂.

In a preferred embodiment of the invention, when Y is equal to 1, R₄ is preferably NO₂.

In a further preferred embodiment of the invention, when Y is equal to 0, R₄ is preferably NH₂.

Advantageously, the invention relates to a compound of formula (IV) selected from the group consisting of:
N-(1,2,3,5,6,7-hexahydro-s-indacen-4-yl)-1-((4-nitrophenyl)sulfonyl)pyrrolidine-2-carboxamide (3a of Formula (IV))

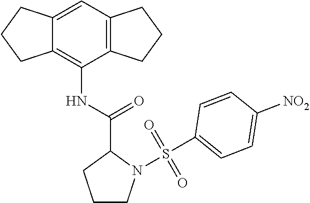 [SMILES: O=C(Nc1c2c(cc3c1CCC3)CCC2)C1CCCN1S(=O)(=O)c1ccc([N+](=O)[O-])cc1]

1-((4-aminophenyl)sulfonyl)-N-(1,2,3,5,6,7-hexahydro-s-indacen-4-yl)piperidine-2-carboxamide (4b of Formula (IV))

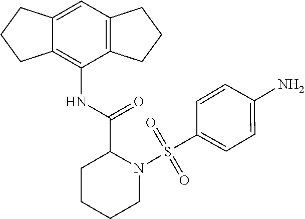 [SMILES: Nc1ccc(S(=O)(=O)N2CCCCC2C(=O)Nc2c3c(cc4c2CCC4)CCC3)cc1]

N-(1,2,3,5,6,7-hexahydro-s-indacen-4-yl)-1-((4-nitrophenyl)sulfonyl)piperidine-2-carboxamide (3b of Formula (IV))

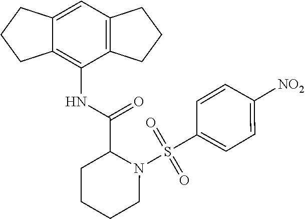 [SMILES: O=C(Nc1c2c(cc3c1CCC3)CCC2)C1CCCCN1S(=O)(=O)c1ccc([N+](=O)[O-])cc1]

1-((4-aminophenyl)sulfonyl)-N-(1,2,3,5,6,7-hexahydro-s-indacen-4-yl)pyrrolidine-2-carboxamide (4a of Formula (IV))

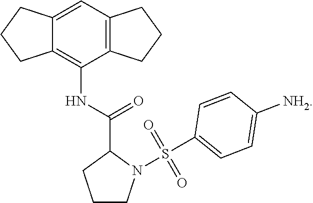 [SMILES: Nc1ccc(S(=O)(=O)N2CCCC2C(=O)Nc2c3c(cc4c2CCC4)CCC3)cc1]

Preferably the compound of the invention of Formula (IV) is selected from the group consisting of:
N-(1,2,3,5,6,7-hexahydro-s-indacen-4-yl)-1-((4-nitrophenyl)sulfonyl)piperidine-2-carboxamide (3b of Formula (IV))

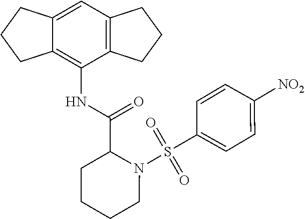 [SMILES: O=C(Nc1c2c(cc3c1CCC3)CCC2)C1CCCCN1S(=O)(=O)c1ccc([N+](=O)[O-])cc1]

1-((4-aminophenyl)sulfonyl)-N-(1,2,3,5,6,7-hexahydro-s-indacen-4-yl)pyrrolidine-2-carboxamide (4a of Formula (IV))

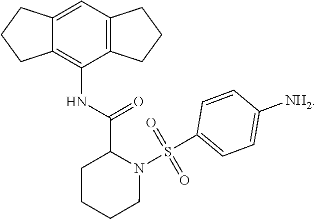 [SMILES: Nc1ccc(S(=O)(=O)N2CCCCC2C(=O)Nc2c3c(cc4c2CCC4)CCC3)cc1]

The compound of formula (I) can be in the form of a pharmaceutically acceptable salt thereof. Such pharmaceutically acceptable salts are preferably selected from the group consisting of hydrochloride, hydrobromide, sulfate, phosphate, acetate, succinate, oxalate, ascorbate, tartrate, gluconate, benzoate, maleate, fumarate and stearate.

The invention also includes a compound of formula (I) labeled with at least one radioisotope such as, for example, tritium (³H), carbon (¹⁴C), iodine (¹²⁵I) or with fluorescent probes, PET (Positron Emission Tomography) or SPECT (Single Photon Emission Tomography).

In another aspect, the invention relates to an hexahydro-s-indacene compound of formula (I) or a pharmaceutically acceptable salt thereof for use as a medicament.

In another aspect, the invention relates to a composition comprising an hexahydro-s-indacene compound of formula (I) or a pharmaceutically acceptable salt thereof in association with pharmaceutically acceptable additives.

For therapeutic application purposes, the compounds described therein can therefore be suitably formulated for administration to mammals (in particular to humans), as such or associated in a suitable pharmaceutical composition with one or more pharmaceutically acceptable excipients and/or carriers. The compositions of the invention include those intended for oral, nasal, sublingual and, in particular, parenteral (subcutaneous, intramuscular, intravenous and intradermal) administration in the form of aqueous and non-aqueous sterile injectable preparations (solutions or suspensions).

Preferably, therefore, the composition of the invention comprises pharmaceutically acceptable carriers and excipients suitable for the final desired formulation according to the possible and desired routes of administration.

Pharmaceutically acceptable additives can be excipients, binders, dispersing agents, colorants, humectants commonly used for the preparation of tablets, capsules, pills, solutions, suspensions, emulsions for oral administration.

In another aspect, the invention relates to an hexahydro-s-indacene compound of formula (I) or its pharmaceutically acceptable salt for use as a selective inhibitor of the NLRP3 inflammasome, preferably in the treatment of pathologies related to hyperactivation of NLRP3 and hyperproduction of interleukin 1β and interleukin 18.

The hexahydro-s-indacene compound is therefore suitable for modulating NLRP3 activity in mammalian cells and tissues in the treatment of NLRP3-related pathologies.

In view of the biological activity profile shown by the hexahydro-s-indacene compound of formula (I) of the present invention, the compound itself, the pharmaceutical compositions comprising it and all the pharmaceutical formulations comprising them can be used for the treatment of pathologies and disorders or conditions associated with the need to reduce the inflammatory state promoted by NLRP3 overactivation including, but not limited to, cancer, metabolic disorders, neurodegenerative diseases, migraine, wound repair and autoimmune diseases.

The hexahydro-s-indacene compound of formula (I) of the invention is preferably in a dose in the range of 50 to 3000 mg per administration unit.

The hexahydro-s-indacene compound of the invention and the compositions according to the invention can be used alone or in combination with other drugs, preferably in a combination therapy in the treatment of NLRP3 related pathologies.

The invention will now be exemplified with reference to examples of the preparation of the compounds of formula (I) and of the evaluation of the therapeutic/medical effects of the compounds by way of example and not of limitation.

### Experimental Section

Preparation and evaluation of Compounds of Formula (II)

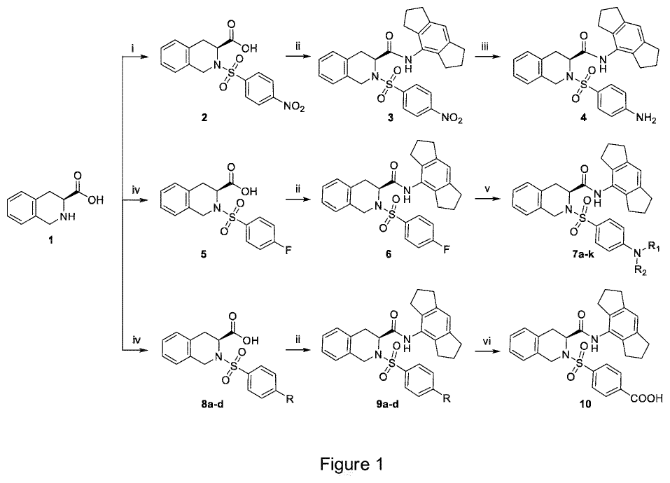

The compounds of Formula (II) were prepared according to the scheme shown in FIG. 1. Compounds of formula (II) were obtained from (S)-1,2,3,4-tetrahydroisoquinolin-3-carboxylic acid 1 which was reacted with appropriate benzenesulfonyl chlorides in the presence of NaHCO₃ (to obtain the intermediate 2) or K₂CO₃ (for the synthesis of compounds 5 and 8a-d) as depicted in FIG. 1. Subsequent amide coupling with hexahydro-s-indacen-4-amine in the presence of HATU and DIPEA provided compounds 3, 6 and 9a-d. Compound 3 was reduced by catalytic hydrogenation to the corresponding aniline derivative 4. Compounds of formula 7a-k were synthesized from 6 by aromatic nucleophilic substitution with appropriate amines in DMSO. Compound 10 was obtained from 5 derivative 9 d by saponification of the ester group in the presence of LiOH.

### Example 1: Preparation of Compound 2 of Formula (II)

To a solution of (S)-1,2,3,4-tetrahydroisoquinolin-3-carboxylic acid 1 (1.0 mmol) in water (5 mL) sodium hydrogen carbonate (NaHCO₃, 2.5 mmol) was added under vigorous stirring. Then 4-nitrobenzenesulfonyl chloride (1.0 mmol) was added in small portions over 1 h and the reaction was stirred at room temperature for 16 h. Next, the reaction mixture was acidified to pH 2 using 1M HCl and the aqueous phase was extracted with ethyl acetate (3×15 mL). The combined organic layers were washed with brine (1×10 mL), dried over Na₂SO₄ and the solvent was evaporated under reduced pressure. The residual crude was crystallized to yield the desired product.

(S)-2-((4-nitrophenyl)sulfonyl)-1,2,3,4-tetrahydroisoquinoline-3-carboxylic acid (2). 48% yield. ¹H NMR (400 MHz, DMSO-d₆): δ 12.97 (bs, 1H), 8.40-8.33 (m, 2H), 8.16-8.09 (m, 2H), 7.15 (q, J=3.6 Hz, 4H), 4.93 (dd, J=6.2, 3.2 Hz, 1H), 4.66 (d, J=15.8 Hz, 1H), 4.46 (d, J=15.8 Hz, 1H), 3.17-3.05 (m, 2H).

### Example 2: Preparation of Compounds 5 and 8a-d of Formula (II)

To a solution of (S)-1,2,3,4-tetrahydroisoquinolin-3-carboxylic acid 1 (5.64 mmol, 1 equiv.) in THF (10 ml) was added H₂O (36 ml) and K₂CO₃ (11.28 mmol, 2 equiv.). A solution of the appropriate benzenesulfonyl chloride (5.64 mmol, 1 eq.) in THF (2 ml) was then added to the mixture. The reaction was allowed to proceed at room temperature for 2 h while maintaining magnetic stirring. The reaction mixture was acidified to pH 4-5 with 1 M HCl and extracted with ethyl acetate (2×15 ml). After drying with Na₂SO₄, the solvent was evaporated to give a solid residue that was purified by flash chromatography using dichloromethane and MeOH as the eluent mixture.

(S)-2-((4-fluorophenyl)sulfonyl)-1,2,3,4-tetrahydroisoquinoline-3-carboxylic acid (5). 54% yield. ¹H NMR (400 MHz, CDCl₃): δ 7.86-7.78 (m, 2H), 7.20-7.00 (m, 6H), 4.95 (t, J=4.7 Hz, 1H), 4.64 (d, J=15.5 Hz, 1H), 4.44 (d, J=15.5 Hz, 1H), 3.17 (d, J=4.7 Hz, 2H).

### (S)-2-((4-(trifluoromethyl)phenyl)sulfonyl)-1,2,3,4-tetrahydroisoquinoline-3-carboxylic acid (8a) of Formula (II)

30% yield. ¹H NMR (400 MHz, CDCl₃) δ 7.94 (d, J=8.2 Hz, 2H), 7.72 (d, J=8.3 Hz, 2H), 7.20-7.11 (m, 2H), 7.10-6.99 (m, 2H), 4.99 (t, J=4.3 Hz, 1H), 4.69 (d, J=15.4 Hz, 1H), 4.44 (d, J=15.4 Hz, 1H), 3.19 (s, 2H). MS (ESI): m/z calculated for C₁₇H₁₃F₃NO₄S [M−H]⁻ 384.06; found, 384.54.

### (S)-2-((4-methoxyphenyl)sulfonyl)-1,2,3,4-tetrahydroisoquinoline-3-carboxylic acid (8b) of Formula (II)

18% yield. ¹H NMR (400 MHz, CDCl₃) δ 7.68 (d, J=8.8 Hz, 2H), 7.03-6.97 (m, 2H), 6.94 (d, J=4.4 Hz, 1H), 6.91-6.88 (m, 1H), 6.81 (d, J=8.8 Hz, 2H), 4.68 (s, 1H), 4.50-4.38 (m, 2H), 3.76 (s, 3H), 3.06 (d, J=14.9 Hz, 1H), 2.92-2.84 (m, 1H). MS (ESI): m/z calculated for C₁₇H₁₈NO₅S [M+H]⁺ 348.08; found, 348.30.

### (S)-2-((4-cyanophenyl)sulfonyl)-1,2,3,4-tetrahydroisoquinoline-3-carboxylic acid (8c) of Formula (II)

68% yield. ¹H NMR (400 MHz, CDCl₃) δ 7.95-7.91 (m, 2H), 7.77-7.73 (m, 2H), 7.22-7.14 (m, 2H), 7.11-7.03 (m, 2H), 5.00 (dd, J=5.5, 3.9 Hz, 1H), 4.71 (d, J=15.3 Hz, 1H), 4.44 (d, J=15.3 Hz, 1H), 3.27-3.16 (m, 2H). MS (ESI): m/z calculated for C₁₇H₁₃N₂O₄S [M−H]⁻ 341.07; found, 341.26.

### (S)-2-((4-(methoxycarbonyl)phenyl)sulfonyl)-1,2,3,4-tetrahydroisoquinoline-3-carboxylic acid (8 d) of Formula (II)

65% yield. ¹H NMR (400 MHz, CDCl₃) δ 8.15-8.11 (m, 2H), 7.92-7.87 (m, 2H), 7.20-7.11 (m, 2H), 7.09-7.02 (m, 2H), 5.01 (t, J=4.7 Hz, 1H), 4.70 (d, J=15.5 Hz, 1H), 4.47 (d, J=15.4 Hz, 1H), 3.97 (s, 3H), 3.19 (d, J=4.6 Hz, 2H). MS (ESI): m/z calculated for C₁₈H₁₈NO₆S [M+H]+ 376.08; found, 376.34.

### Example 3: Preparation of Compounds 3, 6 and 9a-d of Formula (II)

To an ice-cooled solution of 2, 5 or 8a-d (1.1 mmol) in DMF (5 mL) HATU (1.1 mmol) and DIPEA (1.1 mmol) were added. Then, a solution of hexahydro-s-indacen-4-amine (1.0 mmol) in DMF (2 mL) was added dropwise. The resulting mixture was warmed to room temperature and left stirring for 2-5 h. After the removal of the solvent, the crude was dissolved with ethyl acetate (20 mL) and the organic layer was sequentially washed with 10% aqueous solution of citric acid (1×10 mL), 5% aqueous solution of NaHCO₃ (1×10 mL) and brine (1×10 mL). After drying over Na₂SO₄, the solvent was evaporated to yield a solid residue which was firstly triturated with diethyl ether, then filtered and the solid was recrystallized from methanol to give the desired derivatives.

Compound 3 of Formula (II). 84% yield. ¹H NMR (400 MHz, DMSO-d₆): δ9.47 (s, 1H), 8.39-8.31 (m, 2H), 8.16-8.08 (m, 2H), 7.21-7.12 (m, 4H), 6.90 (s, 1H), 4.79 (t, J=5.3 Hz, 1H), 4.74-4.60 (m, 2H), 3.13 (t, J=5.8 Hz, 2H), 2.75 (t, J=7.3 Hz, 4H), 2.47-2.34 (m, 4H), 1.90-1.83 (m, 4H). ¹³C NMR (DMSO-d₆): δ 167.51, 149.60, 143.64, 142.64, 137.39, 132.24, 131.65, 128.81, 128.65, 127.90, 126.76, 126.32, 125.97, 124.35, 117.89, 54.88, 45.35, 40.02, 39.81, 39.60, 39.39, 39.18, 38.98, 38.77, 32.44, 32.25, 29.82, 24.82. MS (ESI): m/z calculated for C₂₈H₂₈N₃O₅S [M+H]⁺ 518.61; found, 518.60.

Compound 6 of Formula (II). 78% yield. ¹H NMR (400 MHz, DMSO-d₆): δ9.40 (s, 1H), 7.94-7.83 (m, 2H), 7.39-7.28 (m, 2H), 7.18-7.06 (m, 4H), 6.89 (s, 1H), 4.65 (t, J=5.5 Hz, 1H), 4.61-4.54 (m, 2H), 3.10-2.97 (m, 2H), 2.74 (t, J=7.3 Hz, 4H), 2.46-2.34 (m, 4H), 1.90-1.83 (m, 4H). ¹³C NMR (DMSO-d₆): δ 168.46, 166.13, 143.19, 137.99, 135.08, 133.18, 132.57, 130.73, 130.63, 129.49, 128.28, 127.30, 126.82, 126.53, 118.40, 116.91, 116.68, 55.51, 45.84, 32.85, 32.76, 30.39, 25.44. MS (ESI): m/z calculated for C₂₈H₂₈FN₂O₃S [M+H]⁺ 491.60; found, 491.60.

Compound 9a of Formula (II). 45% yield. ¹H NMR (400 MHz, CDCl₃) δ 7.97 (d, J=8.2 Hz, 2H), 7.94 (s, 1H), 7.76 (d, J=8.2 Hz, 2H), 7.23-7.15 (m, 2H), 7.14-7.09 (m, 2H), 6.96 (s, 1H), 4.68 (d, J=14.3 Hz, 1H), 4.64 (dd, J=6.4, 4.0 Hz, 1H), 4.32 (d, J=14.2 Hz, 1H), 3.34 (dd, J=15.4, 4.0 Hz, 1H), 2.83 (t, J=7.4 Hz, 4H), 2.70 (dd, J=15.3, 6.4 Hz, 1H), 2.60-2.39 (m, 4H), 2.05-1.92 (m, 4H). ¹³C NMR (CDCl₃) δ 167.79, 144.15, 140.60, 139.03, 137.74, 133.18, 131.86, 128.55, 128.49, 128.35, 127.96, 127.33, 126.66, 126.63, 126.43, 119.25, 57.41, 46.82, 33.06, 30.98, 30.49, 25.65. MS (ESI): m/z calculated for C₂₉H₂₈F₃N₂O₃S [M+H]⁺ 541.17; found, 541.72.

Compound 9b of Formula (II). 25% yield. ¹H NMR (400 MHz, CDCl₃) δ 8.04 (s, 1H), 7.84-7.79 (m, 2H), 7.22-7.11 (m, 4H), 7.01-6.96 (m, 2H), 6.94 (s, 1H), 4.68 (d, J=14.0 Hz, 1H), 4.61 (dd, J=6.2, 3.3 Hz, 1H), 4.22 (d, J=13.9 Hz, 1H), 3.87 (s, 3H), 3.35 (dd, J=15.2, 3.3 Hz, 1H), 2.81 (t, J=7.3 Hz, 4H), 2.64-2.56 (m, 1H), 2.54-2.34 (m, 4H), 2.00-1.91 (m, 4H). ¹³C NMR (CDCl₃) δ 168.37, 163.68, 144.76, 144.02, 141.38, 137.75, 132.40, 133.59, 130.04, 128.47, 128.25, 127.23, 126.47, 119.06, 114.73, 57.34, 55.84, 46.80, 33.06, 31.06, 30.44, 25.65. MS (ESI): m/z calculated for C₂₉H₃₁N₂O₄S [M+H]+ 503.19; found, 503.52.

Compound 9c of Formula (II). 48% yield. ¹H NMR (400 MHz, CDCl₃) δ 7.97-7.92 (m, 2H), 7.89 (s, 1H), 7.80-7.76 (m, 2H), 7.24-7.16 (m, 2H), 7.15-7.09 (m, 2H), 6.97 (s, 1H), 4.67 (d, J=14.5 Hz, 1H), 4.60 (dd, J=6.5, 4.2 Hz, 1H), 4.36 (d, J=14.5 Hz, 1H), 3.33 (dd, J=15.5, 4.3 Hz, 1H), 2.83 (t, J=7.4 Hz, 4H), 2.74 (dd, J=15.4, 6.5 Hz, 1H), 2.61-2.41 (m, 4H), 2.04-1.93 (m, 4H). ¹³C NMR (CDCl₃) δ 167.59, 144.19, 141.30, 137.71, 133.17, 133.02, 131.75, 128.79, 128.57, 128.43, 127.89, 127.36, 126.40, 119.31, 117.24, 117.12, 57.35, 46.80, 33.05, 30.93, 30.50, 25.65. MS (ESI): m/z calculated for C₂₉H₂₈N₃O₃S [M+H]⁺ 498.18; found, 498.69.

Compound 9d of Formula (II). 53% yield. ¹H NMR (400 MHz, CDCl₃) δ 8.17 (d, J=8.2 Hz, 2H), 7.93 (d, J=8.2 Hz, 3H), 7.21-7.08 (m, 4H), 6.95 (s, 1H), 4.70 (d, J=14.1 Hz, 1H), 4.64 (dd, J=6.3, 3.6 Hz, 1H), 4.29 (d, J=14.2 Hz, 1H), 3.97 (s, 3H), 3.34 (dd, J=15.3, 3.6 Hz, 1H), 2.82 (t, J=7.4 Hz, 4H), 2.62 (dd, J=15.3, 6.5 Hz, 1H), 2.57-2.37 (m, 4H), 2.01-1.92 (m, 4H). ¹³C NMR (CDCl₃) δ 167.89, 165.52, 144.11, 140.79, 137.76, 134.68, 133.22, 131.91, 130.69, 128.56, 128.44, 127.99, 127.87, 127.33, 126.44, 119.20, 57.37, 52.91, 46.82, 33.05, 30.96, 30.47, 25.65. MS (ESI): m/z calculated for C₃₀H₃₁N₂O₅S [M+H]+ 531.19; found, 531.65.

### Example 4: Preparation of Compound 4 of Formula (II)

The nitro derivative 3 (1 mmol) was dissolved in ethyl acetate (15 mL) and glacial CH₃COOH (0.5 mL). A catalytic amount (0.1 mmol) of palladium on activated charcoal (10% Pd basis) was added under a hydrogen atmosphere. After 16 h, the reaction mixture was filtered through Celite. The filtrate was concentrated under reduced pressure and the crude was purified via semi-preparative HPLC to give compound 4.

Compound 4 of Formula (II). 82% yield. ¹H NMR (400 MHz, DMSO-d₆): δ 9.24 (s, 1H), 7.49-7.39 (m, 2H), 7.21-7.05 (m, 4H), 6.89 (s, 1H), 6.58-6.48 (m, 2H), 4.63-4.53 (m, 2H), 4.40 (d, J=15.1 Hz, 1H), 3.04 (dd, J=15.6, 4.1 Hz, 1H), 2.85 (dd, J=15.6, 6.2 Hz, 1H), 2.74 (t, J=7.3 Hz, 4H), 2.46-2.33 (m, 4H), 1.93-1.80 (m, 4H). ¹³C NMR (DMSO-d₆): δ 168.88, 153.40, 143.12, 138.03, 133.56, 132.84, 129.61, 128.26, 127.17, 126.78, 126.59, 123.27, 118.31, 113.19, 55.39, 45.70, 32.86, 32.46, 30.36, 25.46. MS (ESI): m/z calculated for C₂₈H₃₀N₃O₃S [M+H]+ 488.63; found, 488.63.

### Example 5: Preparation of Compounds 7a-k of Formula (II)

To a solution of compound 6 (0.10 mmol, 1 eq.) in DMSO (0.5 mL) the appropriate amine (0, 51 mmol, 5 eq.) was added and the mixture was stirred at 120° C. until completion of the reaction (4-16 h). Then H₂O (5 ml) was added, and the aqueous phase was extracted with ethyl acetate (3×5 mL). The organic layers were washed with H₂O (3×5 ml) and brine (10 mL). After drying over Na₂SO₄, the solvent was evaporated to yield a residue which was purified by flash chromatography using an eluent mixture of ethyl acetate and petroleum ether or DCM/MeOH. When necessary, the compounds were further purified by semi-preparative HPLC.

Compound 7a. of Formula (II). 81% yield. ¹H NMR (400 MHz, CDCl₃) δ 8.09 (s, 1H), 7.68-7.64 (m, 2H), 7.40-7.27 (m, 5H), 7.23-7.09 (m, 4H), 6.93 (s, 1H), 6.66 (d, J=8.6 Hz, 2H), 4.65 (d, J=13.9 Hz, 1H), 4.60 (dd, J=6.2, 3.1 Hz, 1H), 4.37 (s, 2H), 4.19 (d, J=13.9 Hz, 1H), 3.35 (dd, J=15.1, 3.2 Hz, 1H), 2.80 (t, J=7.4 Hz, 4H), 2.60 (dd, J=15.0, 6.2 Hz, 1H), 2.54-2.34 (m, 4H), 1.98-1.92 (m, 4H). ¹³C NMR (CDCl₃) δ 168.61, 149.28, 143.98, 141.55, 141.43, 137.77, 134.76, 133.77, 132.66, 129.98, 129.06, 128.42, 128.19, 128.11, 128.00, 127.72, 127.65, 127.26, 127.16, 126.48, 125.50, 118.98, 118.90, 57.29, 46.76, 31.10, 30.43, 25.65. MS (ESI): m/z calculated for C₃₅H₃₆N₃O₃S [M+H]⁺ 578.24; found, 578.74.

Compound 7b of Formula (II). 57% yield. ¹H NMR (400 MHz, CDCl₃) δ 8.11 (s, 1H), 7.74-7.69 (m, 2H), 7.23-7.10 (m, 4H), 6.93 (s, 1H), 6.72-6.67 (m, 2H), 4.67 (d, J=13.8 Hz, 1H), 4.63 (dd, J=6.2, 2.9 Hz, 1H), 4.18 (d, J=13.7 Hz, 1H), 3.36 (dd, J=15.1, 3.0 Hz, 1H), 3.06 (s, 6H), 2.80 (t, J=7.4 Hz, 4H), 2.58 (dd, J=14.9, 6.2 Hz, 1H), 2.53-2.32 (m, 4H), 2.00-1.90 (m, 4H). ¹³C NMR (CDCl₃) δ 168.69, 143.94, 137.76, 133.84, 132.73, 129.70, 129.61, 128.40, 128.21, 128.07, 127.14, 126.48, 121.96, 118.94, 111.41, 65.99, 57.29, 46.76, 40.29, 33.04, 31.14, 30.41, 25.64. MS (ESI): m/z calculated for C₃₀H₃₄N₃O₃S [M+H]⁺ 516.22; found, 516.52.

Compound 7c of Formula (II). 74% yield. ¹H NMR (400 MHz, CDCl₃) δ 8.08 (s, 1H), 7.70 (d, J=9.0 Hz, 2H), 7.21-7.10 (m, 4H), 6.94-6.87 (m, 3H), 4.68-4.60 (m, 2H), 4.19 (d, J=13.8 Hz, 1H), 3.39-3.31 (m, 5H), 2.80 (t, J=7.3 Hz, 4H), 2.60 (dd, J=15.1, 6.3 Hz, 1H), 2.54-2.32 (m, 4H), 2.00-1.89 (m, 4H), 1.72-1.65 (m, 6H). ¹³C NMR (CDCl₃) δ 143.98, 137.77, 137.13, 133.82, 132.68, 129.74, 128.84, 128.43, 128.20, 128.11, 127.17, 126.49, 118.97, 115.08, 114.15, 57.31, 48.88, 46.77, 33.06, 31.13, 30.43, 25.65, 25.33, 24.29. MS (ESI): m/z calculated for C₃₃H₃₈N₃O₃S [M+H]⁺ 556.26; found, 556.73.

Compound 7d. 93% yield. ¹H NMR (400 MHz, CDCl₃) δ 8.05 (s, 1H), 7.77-7.73 (m, 2H), 7.21-7.09 (m, 4H), 6.94 (d, J=9.2 Hz, 3H), 4.67 (d, J=13.9 Hz, 1H), 4.62 (dd, J=6.2, 3.1 Hz, 1H), 4.20 (d, J=13.9 Hz, 1H), 3.90-3.85 (m, 4H), 3.36 (dd, J=15.1, 3.1 Hz, 1H), 3.33-3.28 (m, 4H), 2.80 (t, J=7.3 Hz, 4H), 2.60 (dd, J=15.0, 6.3 Hz, 1H), 2.54-2.33 (m, 4H), 1.99-1.90 (m, 4H). ¹³C NMR (CDCl₃) b 168.49, 154.06, 144.01, 137.76, 133.69, 132.53, 129.71, 128.45, 128.18, 128.14, 127.22, 126.48, 125.77, 119.03, 114.27, 66.49, 57.33, 47.76, 46.79, 33.05, 31.13, 30.43, 25.65. MS (ESI): m/z calculated for C₃₂H₃₆N₃O₄S [M+H]⁺ 558.23; found, 558.76.

Compound 7e of Formula (II). 60% yield. ¹H NMR (400 MHz, CDCl₃) δ 8.13 (s, 1H), 7.69-7.64 (m, 2H), 7.21-7.10 (m, 4H), 6.92 (s, 1H), 6.65 (d, J=9.1 Hz, 2H), 4.69-4.60 (m, 2H), 4.20 (d, J=13.7 Hz, 1H), 3.43-3.37 (m, 4H), 3.34 (d, J=3.1 Hz, 1H), 2.80 (t, J=7.4 Hz, 4H), 2.64 (dd, J=15.0, 6.2 Hz, 1H), 2.54-2.34 (m, 4H), 2.00-1.89 (m, 4H), 1.19 (t, J=7.1 Hz, 6H). ¹³C NMR (CDCl₃) b 168.76, 151.03, 143.95, 137.76, 134.14, 133.91, 132.85, 129.99, 128.37, 128.23 128.03, 127.10, 126.49, 118.93, 110.95, 57.30, 46.75, 44.89, 33.05, 31.17, 30.43, 25.65, 12.42. MS (ESI): m/z calculated for C₃₂H₃₈N₃O₃S [M+H]⁺ 544.26; found, 544.82.

Compound 7f of Formula (II). 52% yield. ¹H NMR (400 MHz, CDCl₃) δ 8.07 (s, 1H), 7.78 (d, J=8.9 Hz, 2H), 7.23-7.10 (m, 4H), 6.95-6.89 (m, 3H), 4.67 (d, J=14.0 Hz, 1H), 4.61 (dd, J=6.2, 3.4 Hz, 1H), 4.19 (d, J=13.8 Hz, 1H), 3.56 (s, 4H), 3.31 (s, 5H), 2.80 (t, J=7.4 Hz, 4H), 2.65 (dd, J=15.1, 6.0 Hz, 1H), 2.55-2.34 (m, 4H), 2.00-1.90 (m, 4H). ¹³C NMR (101 MHz, CDCl₃) δ 169.06, 153.10, 144.14, 137.85, 133.48, 132.45, 129.88, 128.41, 128.35, 127.86, 127.45, 127.33, 126.50, 119.30, 115.53, 57.42, 46.82, 45.14, 43.14, 33.03, 31.22, 30.40, 25.64. MS (ESI): m/z calculated for C₃₂H₃₇N₄O₃S [M+H]⁺ 557.25; found, 557.61.

Compound 7g of Formula (II). 95% yield. ¹H NMR (400 MHz, CDCl₃) δ 8.07 (s, 1H), 7.72 (d, J=9.0 Hz, 2H), 7.22-7.10 (m, 4H), 6.94-6.86 (m, 3H), 4.70-4.58 (m, 2H), 4.19 (d, J=13.8 Hz, 1H), 3.45-3.30 (m, 5H), 2.80 (t, J=7.3 Hz, 4H), 2.70-2.55 (m, 5H), 2.54-2.30 (m, 7H), 2.01-1.87 (m, 4H). ¹³C NMR (CDCl₃) δ 168.55, 154.06, 143.98, 137.77, 133.73, 132.58, 129.70, 128.75, 128.44, 128.17, 127.43, 127.19, 126.48, 119.00, 114.23, 57.33, 54.58, 47.03, 46.78, 45.94, 33.05, 31.14, 30.43, 25.65. MS (ESI): m/z calculated for C₃₃H₃₉N₄O₃S [M+H]⁺ 571.27; found, 571.79.

Compound 7h of Formula (II). 87% yield. ¹H NMR (400 MHz, CDCl₃) δ 8.09 (s, 1H), 7.74 (d, J=8.8 Hz, 2H), 7.22-7.10 (m, 4H), 6.95-6.87 (m, 3H), 4.70-4.61 (m, 2H), 4.18 (d, J=13.8 Hz, 1H), 3.66-3.52 (m, 4H), 3.38-3.28 (m, 5H), 2.80 (t, J=7.3 Hz, 4H), 2.61 (dd, J=15.0, 6.2 Hz, 1H), 2.50-2.32 (m, 4H), 1.99-1.90 (m, 4H), 1.49 (s, 9H). ¹³C NMR (101 MHz, CDCl₃) δ 168.97, 154.78, 153.95, 144.04, 137.82, 133.63, 132.57, 129.77, 128.42, 128.23, 127.95, 127.25, 126.50, 125.16, 119.15, 114.43, 114.32, 80.59, 57.32, 47.39, 46.80, 33.04, 31.19, 30.39, 28.55, 25.64. MS (ESI): m/z calculated for C₃₇H₄₅N₄O₅S [M+H]⁺ 657.30; found, 657.75.

Compound 7i of Formula (II). 48% yield. ¹H NMR (400 MHz, CDCl₃) δ 8.24 (s, 1H), 7.71-7.66 (m, 2H), 7.24-7.13 (m, 4H), 6.94 (s, 1H), 6.64-6.58 (m, 2H), 4.71-4.65 (m, 2H), 4.11 (d, J=13.5 Hz, 1H), 3.32 (dd, J=15.1, 2.9 Hz, 1H), 3.27-3.18 (m, 2H), 2.79 (t, J=7.5 Hz, 4H), 2.69-2.60 (m, 1H), 2.48-2.28 (m, 4H), 1.94 (p, J=7.7 Hz, 4H), 1.29 (t, J=7.2 Hz, 3H). ¹³C NMR (CDCl₃) δ 170.90, 152.31, 144.16, 138.09, 133.51, 132.84, 130.11, 128.35, 127.42, 127.25, 127.11, 126.57, 122.46, 119.60, 112.14, 57.30, 46.90, 38.16, 32.99, 31.38, 30.22, 25.61, 14.58. MS (ESI): m/z calculated for C₃₀H₃₄N₃O₃S [M+H]⁺ 516.22; found, 516.68.

Compound 7j of Formula (II). 85% yield. ¹H NMR (400 MHz, DMSO-d₆): δ 9.28 (s, 1H), 7.56 (d, J=8.9 Hz, 2H), 7.19-7.07 (m, 4H), 6.89 (s, 1H), 6.62 (d, J=8.9 Hz, 2H), 4.67-4.56 (m, 2H), 4.44-4.40 (m, 1H), 3.31-3.27 (m, 2H), 3.09-3.04 (m, 1H), 2.98-2.82 (m, 3H), 2.73 (t, J=7.3 Hz, 4H), 2.46-2.31 (m, 4H), 1.93-1.79 (m, 4H). ¹³C NMR (DMSO-d₆): δ 168.79, 152.11, 143.13, 138.04, 133.44, 132.71, 129.62, 128.35, 127.17, 126.82, 126.60, 124.51, 118.34, 111.84, 55.32, 45.67, 38.19, 32.86, 32.62, 30.34, 25.45. MS (ESI): m/z calculated for C₃₀H₃₅N₄O₃S [M+H]⁺ 531.69; found, 531.70.

Compound 7k of Formula (II). 55% yield. ¹H NMR (400 MHz, Acetone-d₆): δ 8.55 (s, 1H), 7.67-7.60 (m, 2H), 7.29-7.07 (m, 4H), 6.88 (s, 1H), 6.76-6.67 (m, 2H), 4.70 (d, J=14.7 Hz, 1H), 4.63-4.61 (m, 1H), 4.38 (d, J=14.6 Hz, 1H), 3.83 (t, J=7.0 Hz, 2H), 3.26-3.21 (m, 3H), 2.79-2.70 (m, 5H), 2.56-2.39 (m, 5H), 2.09-2.08 (m, 2H), 2.02-1.85 (m, 5H), 1.78-1.74 (m, 2H). ¹³C NMR (Acetone-d₆): δ 168.09, 152.87, 143.07, 137.59, 133.54, 133.28, 129.52, 127.95, 127.16, 126.55, 126.39, 122.93, 117.94, 111.24, 56.58, 46.90, 45.89, 41.81, 32.53, 30.91, 30.11, 25.49, 25.19, 24.71. MS (ESI): m/z calculated for C₃₂H₃₉N₄O₃S [M+H]⁺ 559.75; found, 559.87.

### Example 6: Preparation of Compound 10 of Formula (II)

To a solution of derivative 9 d (0,062 mmol, 1.0 eq.) in methanol (2.5 mL) a solution of LiOH (0,131 mmol, 2.1 eq.) in H₂O (0.5 mL) was added. The resulting mixture was stirred at r.t. for 2 h. The solvents were evaporated, and the residue was diluted with H₂O (1 mL) and acidified with 1M HCl. Then, the aqueous layer was extracted with EtOAc (3×10 mL) and the collected organic phase was washed with brine (10 ml). After drying with Na₂SO₄, the solvent was evaporated to give a solid residue that was purified by preparative HPLC.

Compound 10 of Formula (II). 56% yield. ¹H NMR (400 MHz, CDCl₃): δ 8.26-8.21 (m, 2H), 8.01-7.97 (m, 3H), 7.23-7.10 (m, 4H), 6.96 (s, 1H), 4.76-4.68 (m, 2H), 4.27 (d, J=14.1 Hz, 1H), 3.34 (dd, J=15.3, 3.6 Hz, 1H), 2.82 (t, J=7.4 Hz, 4H), 2.67 (dd, J=15.4, 6.3 Hz, 1H), 2.56-2.36 (m, 4H), 2.02-1.93 (m, 4H). ¹³C NMR (CDCl₃) δ 168.83, 168.30, 144.20, 141.30, 137.89, 133.79, 133.11, 131.95, 131.27, 128.58, 128.54, 128.04, 127.62, 127.46, 126.50, 119.48, 57.40, 46.91, 33.03, 31.15, 30.40, 25.64. MS (ESI): m/z calculated for C₂₉H₂₉N₂O₅S [M+H]⁺ 517.17; found, 517.65.

### Example 7: Evaluation of the Efficacy and Selectivity of Compounds of Formula (II) in Inhibiting the NLRP3-Mediated Release of IL-13 and IL-18 In Vitro

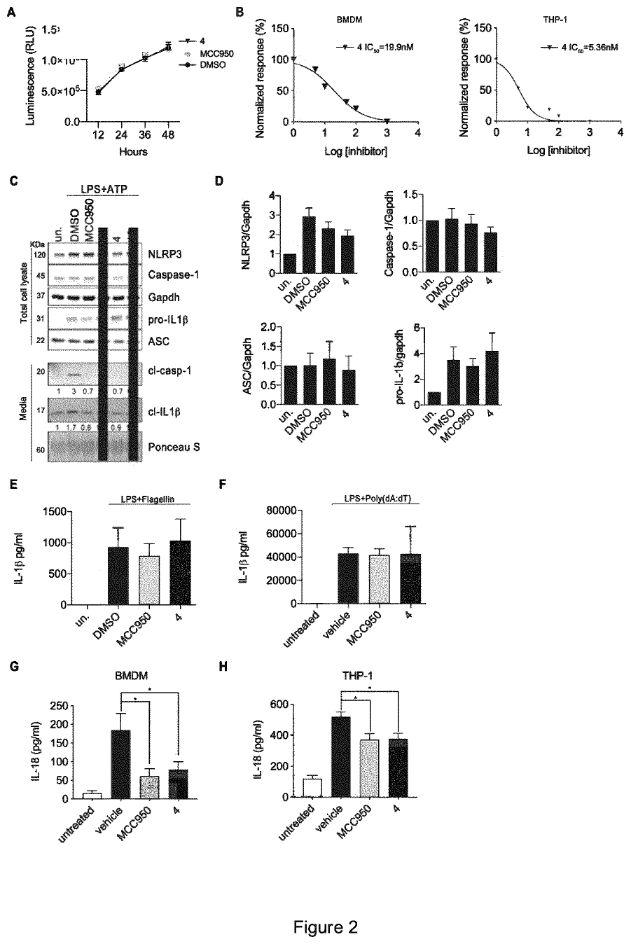

The capability of compounds of Formula (II) to reduce the IL-1β and IL-18 release induced by NLRP3 activation has been investigated in bone marrow-derived macrophages (BMDMs) from wild type (WT) mice first primed with 1 μg/ml lipopolysaccharide (LPS) from Escherichia coli for 2 hours. The compounds of Formula (II) have been added at 1 μM of concentration for 30 minutes and then cells have been stimulated with 2′(3′)-O-(4-benzoylbenzoyl)adenosine 5′-triphosphate triethylammonium salt (Bz-ATP) at 100 μM of concentration for 30 minutes. Cell culture supernatants were analyzed for IL-1β and IL-18 by enzyme-linked immunosorbent assay (ELISA). The potency of the compounds of Formula (II) was expressed as a percentage of inhibition of the release of IL-1β at 1 μM of concentration. The inhibition rates of IL-1β release of compound 4 was: 65.2%. Under the same experimental conditions MCC950 used as internal control showed a percentage of inhibition of 60.1%. IC₅₀ value for compound 4 is 19.9 nM obtained in BMDM stimulated with LPS and ATP (FIG. 2B). The efficacy of compound 4 has been also evaluated in human macrophages (THP-1) stimulated with LPS and ATP: IC₅₀ value obtained in THP-1 cells is 5.36 nM (FIG. 2B).

Accordingly, the NLRP3-stimulated secretion of IL-18 was also significantly reduced after treatment with compound 4 both in BMDM and THP-1 cells (FIG. 2G and H)

The selectivity of compound 4 as an inhibitor of NLRP3 was then evaluated with respect to NLRC4 and absent in melanoma 2 (AIM2) inflammasomes. For activation of NLRC4 inflammasome, BMDM were treated with LPS (100 ng/ml) for 3 hours. Subsequently, the culture medium was removed and replaced with a serum-free medium containing compound 4 (1 μm for 30 minutes), finally the cells were transfected with flagellin (100 ng/ml) by S. typhimurium for 2 hours. The supernatant was tested by ELISA. Compound 4 has no effect on the activation of NLRC4 inflammasome triggered by flagellin S. typhimurium (FIG. 2E), demonstrating specificity in the inhibition of NLRP3 inflammasome. The effect of the same compounds was examined on the non-NLR AIM2 inflammasome by transfecting BMDM with the dsDNS analogue Poly (dA:dT). A reduction in the amount of IL-1β secretion was not observe by the compound of Formula (II) (FIG. 2F)

### Example 8: Evaluation of the Effect of Compound 4 of Formula (II) on Cell Viability of THP-1 Cells

Cell viability was evaluated on THP-1 cells using realtime-Glo” MT Cell Viability Assay (Promega Italia, MI). Briefly, the cells were seeded in a 96 multiwell (10×10⁴ cells/well), treated with the selected compound of Formula (II), and incubated for 48 hours. After treatment, the cells were incubated for 10 minutes in the incubator with the realtime-Glo” reagent following protocol. The luminescence was measured every 12 hours in the Glomax Multi Detection System Promega. The luminescence signal correlates with the number of metabolically active cells.

The results represent the mean luminescence values (RLU)±SEM. Compound 4, at the concentration used, showed no cytotoxicity and therefore did not affect cell viability of THP-1 cells (FIG. 2A).

### Example 9: Evaluation of the Effect of Compound 4 of Formula (II) on the Protein Expression of NLRP3 Inflammasome

Total cell lysates of THP-1 cells were prepared in RIPA buffer (50 mM Tris-HCl pH 7.8, 150 mM NaCl, 1% IGEPAL CA-630, 0.5% sodium deoxycholate, 0.1% SDS, 1 mM dithiothreitol (DTT)) supplemented with proteases and phosphatases inhibitors. Supernatant from THP-1 was concentrated using Pierce Protein Concentrators PES 10K MWCO (ThermoFisher) then was centrifuged at 4000×g for 15 minutes. A total of 20 μg of protein or a total of 10 μl of concentrated 5 medium was separated by SDS-PAGE and transferred to nitrocellulose membranes for standard western blotting. The following primary antibodies were used: NLRP3 (Adipogen, #AG-20B-0014-C100), ASC (Adipogen, #AG-20B-0014-C100) Caspase 1 (Novus Biological, #14F468) and IL-1β (Cell Signaling Technology, #12242), GAPDH (Cell Signaling Technology, #2118). Isotype-matched horseradish peroxidase-conjugated secondary antibodies were used, followed by detection using chemiluminescence (GE Image-Quant). Treatment with compound 4 did not affect the priming phase of activation of NLRP3 inflammasome, not altering the expression of NLRP3, caspase-1, ASC and pro-IL-1β in total cell lysate (FIG. 2C-D). The compound of Formula (II) significantly reduced the release of IL-1β and the amount of caspase-1 cleaved into the cellular supernatant (FIG. 2C).

### Example 10: Evaluation of the Effect of Compound 4 of Formula (II) on LPS-Induced Inflammation In Vivo and Ex Vivo

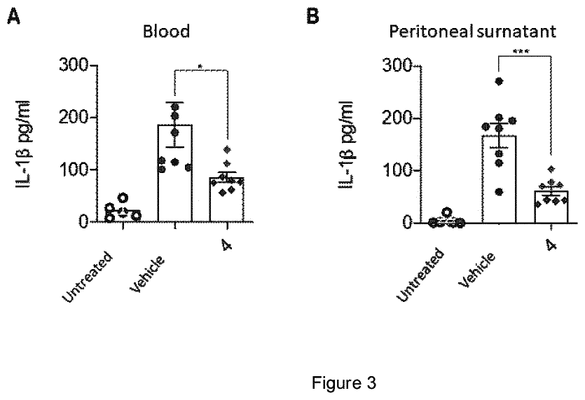

C57BL/6 mice were treated with compound 4 of Formula (II) at a concentration of 25 mg/Kg or vehicle (DMSO), administered by intraperitoneal injection for 30 minutes. The mice were subsequently treated with LPS 1 mg/Kg for 4 hours, administered intraperitoneally. Mice were sacrificed and blood and peritoneal supernatant were collected. The blood was centrifuged (1000×g for 15 minutes) to obtain the plasma. ELISA tests were carried out on plasma and peritoneal supernatant to assess IL-1β levels. Mice receiving treatment with compound 4 of Formula (II) showed a significant reduction in IL-1β release compared to mice treated with the vehicle, indicating that the compound had a strong efficacy in reducing the activation of NLRP3 inflammasome also in vivo (FIG. 3).

### Example 11: Evaluation of the Effect of Compound 4 of Formula (II) on Tumor Growth In Vivo

Procedures involving animals and their care were in conformity with institutional guidelines, and the Animal Ethics Committee approved all experimental protocols (Authorization N^{o} 481/2017-PR and CBCC2.N.BH4 approved by the Italian Ministry of Health). A total of 1×10⁶ B16-F10 melanoma cells transfected with cytluc (B16-F10cytLUC) were subcutaneously inoculated into females, 6- to 8-week-old C57BL/6 mice. Tumor growth was monitored daily, and tumor volumes were measured every other day with calipers using the following equation: Volume=π/6×(a×b2), where “a” is the major diameter and “b” is the minor diameter. As soon as the mass was palpable, mice were randomly divided (5 mice per group) into the treatment groups and the control group, with 5 mice per group. For NLRP3 inflammasome inhibition, mice were intraperitoneal injected with selected molecules thrice weekly at 25 mg/Kg; control mice received equal volumes of DMSO.

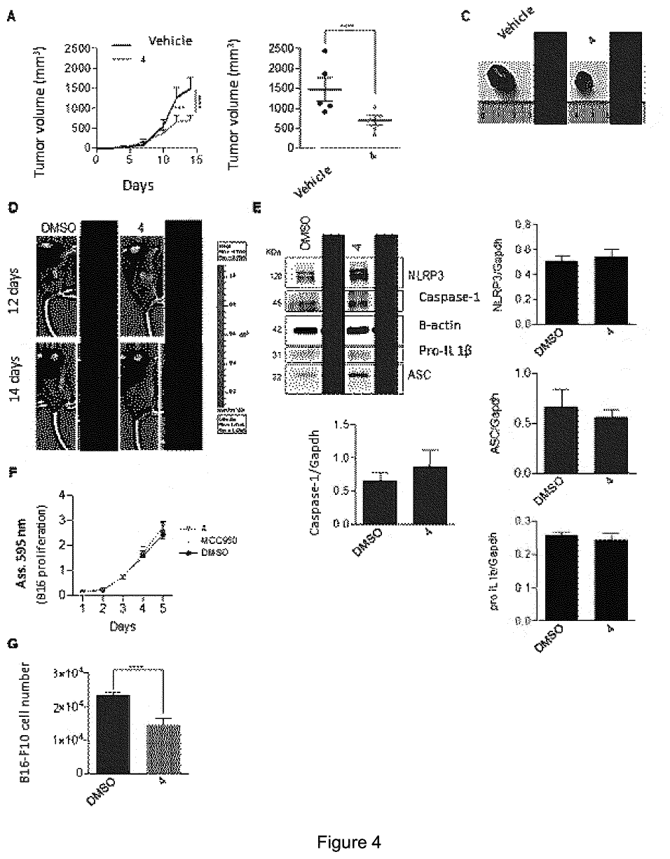

Luciferase luminescence was followed by a total body luminometer (IVIS Lumina, Caliper-PerkinElmer). Briefly, mice were anesthetized with 2.5% isoflurane intraperitoneal injected with 150 mg/Kg d-luciferin (Promega) and luminescence quantified after 15 min using the Living Image Software (Caliper). Mice treated with compound 4 developed a mass that was significantly smaller (about 50% reduction) than mice treated with vehicle (DMSO) (FIG. 4A-D). In the lysates of the extracted tumor masses there was no alteration in the expression of the proteins NLRP3, caspase-1, ASC and pro-IL-1β (FIG. 4E).

### Example 12: B16-F10 Cancer Cells Proliferation after Treatment with Compound 4 of Formula (II)

B16-F10 cells were treated with selected inhibitors (1 μM) then were counted with a Burker chamber and plated in five sets of 4 wells of a 24-well plate. Starting from the following day (day 1) 1 set of wells (at days 2, 3, 4 and 5) was washed once with PBS, fixed in 4% formaldehyde solution for 15 min at room temperature, and then kept in PBS at 4° C. At day 5, all the wells were stained with crystal violet for 20 minutes, lysed with 10% acetic acid, then the absorbance was read at 595 nm. Compound 4 had no direct effect on cell proliferation of B16-F10 melanoma cells (FIG. 4F).

### Example 13: Evaluation of the Effect of Compound 4 of Formula (II) on the Tumor Microenvironment by Co-Culture Model

Co-culture of peritoneal macrophages and B16-F10 cells were performed using Transwell chambers with 0.4-μm pores on the membranes (Corning, Corning, NY, USA). Peritoneal macrophages and B16-F10 cells were cultured in the lower and upper compartments of the Transwell chamber, respectively, for 48 h. Peritoneal macrophages were seeded at a density of 2×10⁵ cells and treated with LPS (1 μg/mL) for 2 hours, then DMSO or compound 4 (1 μM) of Formula (II) for 30 minutes and finally stimulated with ATP for 1 hour. B16-F10 cells were seeded at a density of 2×10³ cells each well. After 48 h, B16-F10 cells in the upper chambers were removed and counted using an automated cell counter (Tali™ image-based cytometer (Invitrogen). B16-F10 cells in contact with peritoneal macrophages treated with the vehicle alone grew more than B16-F10 cells in contact with peritoneal macrophages treated with compound 4 (FIG. 4G). Compound 4 of Formula (II) influenced the tumor microenvironment, reducing the activation of NLRP3 inflammasome.

### Preparation and Evaluation of Compounds of Formula (III)

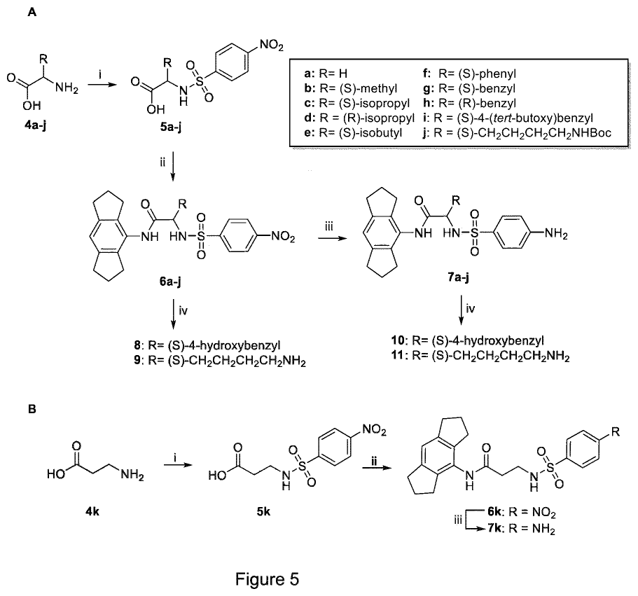

The compounds of Formula (III) of the invention were prepared according to the scheme shown in FIG. 5. Compounds of formula (III) were obtained starting from the a/b amino acids 4a-k that were reacted with 4-nitrobenzenesulfonyl chloride in the presence of NaHCO₃ to give the 5a-k intermediates (FIG. 5). Subsequent amide coupling with hexahydro-s-indacen-4-amine in the presence of HATU e DIPEA provided the 6a-k compounds. The tyrosine (6i) and lysine derivatives (6j) were treated with HCl in dioxane solution to remove the side-chain protections leading to compounds 8 and 9, respectively. The 6a-k compounds were reduced by catalytic hydrogenation to the corresponding 7a-k aniline derivatives. The side-chain protections of 7i-j were also removed under acidic conditions to obtain 10 and 11, respectively.

### Example 14: Preparation of Compounds 5a-k of Formula (III)

To a solution of 4a-k (1.0 mmol) in water (5 mL) sodium hydrogen carbonate (NaHCO₃, 2.5 mmol) was added under vigorous stirring. Then 4-nitrobenzenesulfonyl chloride (1.0 mmol) was added in small portions over 1 h and the reaction was stirred at room temperature for 16 h. Next, the reaction mixture was acidified to pH 2 using 1 M HCl and the aqueous phase was extracted with ethyl acetate (3×15 mL). The combined organic layers were washed with brine (1×10 mL), dried over Na₂SO₄ and the solvent was evaporated under reduced pressure. The residual crude was crystallized to yield the desired products.

((4-nitrophenyl)sulfonyl)glycine (5a) of Formula (III). 60% yield. ¹H NMR (400 MHz, DMSO-d₆): δ 12.66 (bs, 1H), 8.46 (t, J=6.1 Hz, 1H), 8.40-8.34 (m, 2H), 8.06-7.99 (m, 2H), 3.68 (d, J=6.1 Hz, 2H).

((4-nitrophenyl)sulfonyl)-L-alanine (5b) of Formula (III). 82% yield. ¹H NMR (400 MHz, DMSO-d₆): δ 12.68 (s, 1H), 8.55 (s, 1H), 8.40-8.34 (m, 2H), 8.05-7.99 (m, 2H), 3.89-3.83 (m, 1H), 1.18 (d, J=7.2 Hz, 3H).

((4-nitrophenyl)sulfonyl)-L-valine (5c) of Formula (III). 60% yield. ¹H NMR (400 MHz, DMSO-d₆): δ 12.63 (bs, 1H), 8.43 (d, J=9.5 Hz, 1H), 8.41-8.34 (m, 2H), 8.06-7.99 (m, 2H), 3.59 (dd, J=9.5, 5.9 Hz, 1H), 2.02-1.90 (m, 1H), 0.80 (dd, J=14.0, 6.8 Hz, 6H).

((4-nitrophenyl)sulfonyl)-D-valine (5 d) of Formula (III). 52% yield. ¹H NMR (400 MHz, DMSO-d₆): δ 12.63 (bs, 1H), 8.45 (d, J=9.5 Hz, 1H), 8.41-8.36 (m, 2H), 8.05-8.00 (m, 2H), 3.61 (dd, J=9.5, 5.9 Hz, 1H), 2.05-1.92 (m, 1H), 0.82 (dd, J=13.9, 6.8 Hz, 6H).

((4-nitrophenyl)sulfonyl)-L-leucine (5e) of Formula (III). 81% yield. ¹H NMR (400 MHz, DMSO-d₆): δ 12.60 (bs, 1H), 8.57 (d, J=9.0 Hz, 1H), 8.42-8.37 (m, 2H), 8.04-8.00 (m, 2H), 3.78-3.72 (m, 1H), 1.63-1.54 (m, 1H), 1.45-1.40 (m, 2H), 0.84 (d, J=6.6 Hz, 3H), 0.75 (d, J=6.5 Hz, 3H).

(S)-2-((4-nitrophenyl)sulfonamido)-2-phenylacetic acid (5f) of Formula (III). 74% yield. ¹H NMR (400 MHz, DMSO-d₆): δ 13.03 (bs, 1H), 9.11 (d, J=9.3 Hz, 1H), 8.29-8.21 (m, 2H), 7.96-7.89 (m, 2H), 7.26-7.18 (m, 5H), 4.98 (d, J=9.0 Hz, 1H).

((4-nitrophenyl)sulfonyl)-L-phenylalanine (5g) of Formula (III). 81% yield. ¹H NMR (400 MHz, DMSO-d₆): δ 12.85 (bs, 1H), 8.70 (d, J=8.5 Hz, 1H), 8.22-8.14 (m, 2H), 7.77-7.67 (m, 2H), 7.15-7.03 (m, 5H), 3.94 (t, J=10.0 Hz, 1H), 2.99-2.95 (m, 1H), 2.72-2.70 (m, 1H).

((4-nitrophenyl)sulfonyl)-D-phenylalanine (5 h) of Formula (III). 78% yield. ¹H NMR (400 MHz, DMSO-d₆): δ 12.82 (bs, 1H), 8.64 (d, J=8.5 Hz, 1H), 8.20-8.11 (m, 2H), 7.75-7.67 (m, 2H), 7.18-7.05 (m, 5H), 3.97-3.89 (m, 1H), 2.99-2.95 (m, 1H), 2.72-2.70 (m, 1H).

(S)-3-(4-(tert-butoxy)phenyl)-2-((4-nitrophenyl)sulfonamido)propanoic acid (5i) of Formula (III). 71% yield. ¹H NMR (400 MHz, DMSO-d₆): 12.84 (bs, 1H), 8.70 (d, J=9.1 Hz, 1H), 8.26-8.17 (m, 2H), 7.77-7.73 (m, 2H), 7.05-6.95 (m, 2H), 6.73-6.63 (m, 2H), 3.96-3.90 (m, 1H), 2.94 (dd, J=13.8, 4.7 Hz, 1H), 2.68-2.65 (m, 1H), 1.21 (s, 9H).

N⁶-(tert-butoxycarbonyl)-N²-((4-nitrophenyl)sulfonyl)-L-lysine (5j) of Formula (III). 80% yield. ¹H NMR (400 MHz, DMSO-d₆): δ 12.62 (bs, 1H), 8.52 (d, J=8.8 Hz, 1H), 8.42-8.33 (m, 2H), 8.04-7.96 (m, 2H), 6.70 (t, J=5.5 Hz, 1H), 3.73-3.68 (m, 1H), 2.80-2.75 (m, 2H), 1.65-1.41 (m, 2H), 1.34 (s, 9H), 1.26-1.22 (m, 4H).

3-((4-nitrophenyl)sulfonamido)propanoic acid (5k) of Formula (III). 69% yield. ¹H NMR (400 MHz, DMSO-d₆): δ 12.26 (s, 1H), 8.43-8.37 (m, 2H), 8.07 (bs, 1H), 8.05-8.00 (m, 2H), 3.00-2.97 (m, 2H), 2.36 (t, J=6.9 Hz, 2H).

### Example 15: Preparation of Compounds 6a-k, 8 and 9 of Formula (III)

To an ice-cooled solution of 5a-k (1.1 mmol) in DMF (5 mL) HATU (1.1 mmol) and DIPEA (1.1 mmol) were added. Then, a solution of hexahydro-s-indacen-4-amine (1.0 mmol) in DMF (2 mL) was added dropwise. The resulting mixture was warmed to room temperature and left stirring for 2-5 h. After the removal of the solvent, the crude was dissolved with ethyl acetate (20 mL) and the organic layer was sequentially washed with 10% aqueous solution of citric acid (1×10 mL), 5% aqueous solution of NaHCO₃ (1×10 mL) and brine (1×10 mL). After drying over Na₂SO₄, the solvent was evaporated to yield a solid residue which was firstly triturated with diethyl ether, then filtered and the solid was recrystallized from methanol to give the desired derivatives. The intermediates 6i and 6j were respectively deprotected by treatment with 4N HCl in dioxane at room temperature. After completion of the reaction, the solvent was removed, and the crudes were purified via semi-preparative HPLC to furnish the unprotected compounds 8 and 9.

Compound 6a of Formula (III). 67% yield. ¹H NMR (400 MHz, DMSO-d₆): δ 9.39 (s, 1H), 8.53 (s, 1H), 8.48-8.36 (m, 2H), 8.14-7.99 (m, 2H), 6.91 (s, 1H), 3.79 (s, 2H), 2.77 (t, J=7.4 Hz, 4H), 2.49-2.43 (m, 4H), 1.93-1.85 (m, 4H). ¹³C NMR (DMSO-d₆): δ 165.12, 149.32, 146.25, 142.71, 137.07, 128.94, 128.20, 124.25, 117.81, 44.78, 32.29, 30.11, 24.89. MS (ESI): m/z calculated for C₂₀H₂₂N₃O₅S [M+H]⁺ 416.47; found, 416.33.

Compound 6b of Formula (III). 77% yield. ¹H NMR (400 MHz, DMSO-d₆): δ 9.48 (s, 1H), 8.68 (d, J=7.5 Hz, 1H), 8.41 (d, J=8.7 Hz, 2H), 8.09 (d, J=8.8 Hz, 2H), 6.91 (s, 1H), 4.18-4.07 (m, 1H), 2.76 (t, J=7.4 Hz, 4H), 2.40 (t, J=7.4 Hz, 4H), 1.88-1.84 (m, 4H), 1.30 (d, J=6.7 Hz, 3H). ¹³C NMR (DMSO-d₆): δ 169.51, 149.82, 147.42, 143.24, 137.71, 129.40, 128.63, 124.83, 118.41, 52.33, 32.82, 30.46, 25.39, 20.63. MS (ESI): m/z calculated for C₂₁H₂₄N₃O₅S [M+H]⁺ 430.50; found, 430.35.

Compound 6c of Formula (III). 79% yield. ¹H NMR (400 MHz, DMSO-d₆): δ 9.38 (s, 1H), 8.45 (bs, 1H), 8.37 (d, J=8.9 Hz, 2H), 8.05 (d, J=8.9 Hz, 2H), 6.87 (s, 1H), 3.80 (d, J=6.2 Hz, 1H), 2.74-2.70 (m, 4H), 2.38-2.19 (m, 4H), 1.98 (dq, J=13.4, 6.6 Hz, 1H), 1.89-1.73 (m, 4H), 0.93 (d, J=6.7 Hz, 3H), 0.85 (d, J=6.8 Hz, 3H). ¹³C NMR (DMSO-d₆): δ 149.58, 143.23, 137.53, 129.46, 128.56, 124.72, 118.35, 62.16, 32.80, 31.86, 30.63, 25.35, 19.79, 18.25. MS (ESI): m/z calculated for C₂₃H₂₈N₃O₅S [M+H]⁺ 458.55; found, 458.28.

Compound 6d of Formula (III). 88% yield. ¹H NMR (400 MHz, DMSO-d₆): δ 9.51 (s, 1H), 8.49 (d, J=7.5 Hz, 1H), 8.43-8.31 (m, 2H), 8.13-8.01 (m, 2H), 6.87 (s, 1H), 3.85 (t, J=6.7 Hz, 1H), 2.71 (t, J=7.3 Hz, 4H), 2.37-2.17 (m, 4H), 2.05-1.93 (m, 1H), 1.86-1.72 (m, 4H), 0.95 (d, J=6.7 Hz, 3H), 0.86 (d, J=6.8 Hz, 3H). ¹³C NMR (DMSO-d₆): δ 168.18, 149.73, 147.66, 143.20, 137.61, 129.44, 128.68, 124.77, 118.37, 62.05, 32.79, 31.81, 30.63, 25.33, 19.74, 18.30. MS (ESI): m/z calculated for C₂₃H₂₈N₃O₅S [M+H]⁺ 458.55; found, 457.70.

Compound 6e of Formula (III). 78% yield. ¹H NMR (400 MHz, DMSO-d₆): δ 9.47 (s, 1H), 8.60 (s, 1H), 8.44-8.35 (m, 2H), 8.09-8.01 (m, 2H), 6.88 (s, 1H), 4.05-4.01 (m, 1H), 2.73 (t, J=7.3 Hz, 4H), 2.32 (t, J=7.4 Hz, 4H), 1.93-1.77 (m, 4H), 1.72-1.64 (m, 1H), 1.55-1.38 (m, 2H), 0.89 (d, J=6.7 Hz, 3H), 0.83 (d, J=6.6 Hz, 3H). ¹³C NMR (DMSO-d₆): δ 169.30, 149.75, 147.61, 143.22, 137.70, 129.40, 128.62, 124.78, 118.41, 55.19, 42.81, 32.81, 30.47, 25.40, 24.49, 23.34, 21.75. MS (ESI): m/z calculated for C₂₄H₃₀N₃O₅S [M+H]⁺ 472.58; found, 472.52.

Compound 6f of Formula (III). 69% yield. ¹H NMR (400 MHz, DMSO-d₆): δ 9.69 (s, 1H), 9.22 (d, J=9.2 Hz, 1H), 8.32 (d, J=8.8 Hz, 2H), 8.02 (d, J=8.7 Hz, 2H), 7.42 (d, J=6.9 Hz, 2H), 7.30-7.22 (m, 3H), 6.89 (s, 1H), 5.30 (d, J=9.2 Hz, 1H), 2.72 (t, J=7.2 Hz, 4H), 2.36-2.12 (m, 4H), 1.91-1.66 (m, 4H). ¹³C NMR (DMSO-d₆): δ 166.43, 149.10, 146.72, 142.75, 137.30, 137.00, 128.53, 128.10, 127.69, 126.90, 124.04, 117.96, 59.39, 32.16, 29.72, 24.79. MS (ESI): m/z calculated for C₂₆H₂₆N₃O₅S [M+H]⁺ 492.57; found, 492.50.

Compound 6g of Formula (III). 84% yield. ¹H NMR (400 MHz, DMSO-d₆): δ 9.54 (s, 1H), 8.82-8.79 (m, 1H), 8.26 (d, J=8.5 Hz, 2H), 7.86 (d, J=8.7 Hz, 2H), 7.21-7.16 (m, 5H), 6.91 (s, 1H), 4.31-4.27 (m, 1H), 3.44-3.37 (m, 1H), 3.00-2.97 (m, 1H), 2.81-2.74 (m, 4H), 2.41-2.27 (m, 4H), 1.88-1.83 (m, 4H). ¹³C NMR (DMSO-d₆): δ 168.62, 149.55, 147.48, 143.25, 137.66, 137.29, 129.82, 129.30, 128.49, 128.24, 126.81, 124.64, 118.41, 58.18, 32.83, 30.47, 25.46. MS (ESI): m/z calculated for C₂₇H₂₈N₃O₅S [M+H]⁺ 506.60; found, 506.50.

Compound 6h. 73% yield. ¹H NMR (400 MHz, DMSO-d₆): δ 9.54 (s, 1H), 8.80 (bs, 1H), 8.26 (d, J=8.6 Hz, 2H), 7.86 (d, J=8.4 Hz, 2H), 7.21-7.17 (m, 5H), 6.91 (s, 1H), 4.32-4.26 (m, 1H), 3.02-2.97 (m, 1H), 2.81-2.74 (m, 5H), 2.42-2.28 (m, 4H), 2.01-1.72 (m, 4H). ¹³C NMR (DMSO-d₆): δ 168.61, 149.55, 147.48, 143.24, 137.66, 129.82, 128.24, 126.81, 124.64, 118.41, 58.18, 32.83, 30.47, 25.46. MS (ESI): m/z calculated for C₂₇H₂₈N₃O₅S [M+H]⁺ 506.60; found, 506.45.

(S)-3-(4-(tert-butoxy)phenyl)-N-(1,2,3,5,6,7-hexahydro-s-indacen-4-yl)-2-((4-nitrophenyl)sulfonamido)propanamide (6i) of Formula (III). 79% yield. ¹H NMR (400 MHz, DMSO-d₆): δ 9.55 (s, 1H), 8.83-8.79 (m, 1H), 8.33-8.25 (m, 2H), 7.94-7.85 (m, 2H), 7.14-7.06 (m, 2H), 6.91 (s, 1H), 6.81-6.73 (m, 2H), 4.29-4.22 (m, 1H), 2.76 (t, J=7.3 Hz, 6H), 2.42-2.29 (m, 4H), 1.91-1.78 (m, 5H), 1.24 (s, 9H). ¹³C NMR (DMSO-d₆): δ 168.09, 153.55, 148.97, 147.05, 142.66, 137.10, 131.15, 129.72, 128.77, 127.67, 127.08, 124.11, 123.01, 117.82, 77.39, 57.75, 38.22, 32.25, 29.92, 28.32, 24.90. MS (ESI): m/z calculated for C₃₁H₃₆N₃O₆S [M+H]⁺ 578.70; found, 578.55.

Tert-butyl (S)-(6-((1,2,3,5,6,7-hexahydro-s-indacen-4-yl)amino)-5-((4-nitrophenyl)sulfonamido)-6-oxohexyl)carbamate (6j) of Formula (III). 83% yield. ¹H NMR (400 MHz, DMSO-d₆): δ 9.45 (s, 1H), 8.60 (bs, 1H), 8.45-8.36 (m, 2H), 8.13-8.03 (m, 2H), 6.90 (s, 1H), 6.76 (t, J=5.8 Hz, 1H), 4.06-3.88 (m, 1H), 2.87-2.82 (m, 2H), 2.75 (t, J=7.3 Hz, 4H), 2.35 (t, J=7.1 Hz, 4H), 1.89-1.81 (m, 4H), 1.75-1.47 (m, 2H), 1.36 (s, 9H), 1.32-1.18 (m, 4H). ¹³C NMR (DMSO-d₆): δ 168.42, 155.40, 149.14, 142.66, 137.06, 128.85, 128.00, 124.21, 117.81, 77.20, 57.26, 56.08, 33.16, 32.23, 29.95, 28.82, 28.14, 24.82, 22.39. MS (ESI): m/z calculated for C₂₉H₃₉N₄O₇S [M+H]⁺ 587.71; found, 587.58, 531.53 [M-tBu]⁺, 487.57 [M-Boc]⁺.

Compound 6k of Formula (III). Yield 89% yield. ¹H NMR (400 MHz, DMSO-d₆): δ 9.36 (s, 1H), 8.43 (d, J=7.4 Hz, 2H), 8.11-8.06 (m, 3H), 6.93 (s, 1H), 3.12-3.06 (m, 2H), 2.79 (t, J=7.5 Hz, 4H), 2.62 (t, J=7.4 Hz, 4H), 2.48-2.43 (m, 2H), 1.96-1.92 (m, 4H). ¹³C NMR (DMSO-d₆): δ 167.92, 150.01, 146.44, 143.18, 137.77, 130.07, 128.56, 125.08, 118.20, 39.66, 36.05, 32.91, 30.80, 25.51. MS (ESI): m/z calculated for C₂₁H₂₄N₃O₅S [M+H]⁺ 430.50; found, 430.43.

Compound 8 of Formula (III). 71% yield. ¹H NMR (400 MHz, DMSO-d₆): δ 9.52 (s, 1H), 8.71 (d, J=9.1 Hz, 1H), 8.27-8.20 (m, 2H), 7.86-7.78 (m, 2H), 7.00-6.93 (m, 2H), 6.89 (s, 1H), 6.57-6.47 (m, 2H), 4.21-4.15 (m, 1H), 2.86 (dd, J=13.7, 5.3 Hz, 1H), 2.74 (t, J=7.3 Hz, 4H), 2.67-2.61 (m, 1H), 2.46-2.25 (m, 4H), 1.91-1.79 (m, 4H). ¹³C NMR (DMSO-d₆): δ 168.27, 155.98, 148.85, 147.02, 142.65, 137.11, 130.14, 128.83, 127.66, 126.69, 124.00, 117.79, 114.62, 58.11, 32.27, 29.94, 24.89. MS (ESI): m/z calculated for C₂₇H₂₈N₃O₆S [M+H]⁺ 522.60; found, 522.62.

Compound 9 of Formula (III). 70% yield. ¹H NMR (400 MHz, DMSO-d₆): δ 9.64 (s, 1H), 8.66 (d, J=9.1 Hz, 1H), 8.43-8.36 (m, 2H), 8.13-8.05 (m, 2H), 7.95 (s, 3H), 6.90 (s, 1H), 4.08-4.03 (m, 1H), 2.74 (t, J=7.4 Hz, 6H), 2.34 (t, J=7.4 Hz, 4H), 1.90-1.77 (m, 4H), 1.75-1.68 (m, 1H), 1.66-1.51 (m, 3H), 1.39-1.29 (m, 2H). ¹³C NMR (DMSO-d₆): δ 168.88, 149.85, 147.52, 143.28, 137.72, 129.45, 128.75, 124.89, 118.46, 56.54, 38.91, 33.43, 32.87, 30.62, 26.77, 25.44, 22.66. MS (ESI): m/z calculated for C₂₄H₃₁N₄O₅S [M+H]⁺ 487.59; found, 487.59.

### Example 16: Preparation of Compounds 7a-k, 10-11 of Formula (III)

The nitro derivatives 6a-k (1 mmol) were dissolved in ethyl acetate (15 mL) and glacial CH₃COOH (0.5 mL). A catalytic amount (0.1 mmol) of palladium on activated charcoal (10% Pd basis) was added under hydrogen atmosphere. After 16 h, the reaction mixture was filtered through Celite. The filtrate was concentrated under reduced pressure and the crudes were purified via semi-preparative HPLC to give compounds 7a-k. Derivatives 7i-j were deprotected as described for 6i-j to yield the final compounds 10 and 11.

Compound 7a of Formula (III). 91% yield. ¹H NMR (400 MHz, DMSO-d₆): δ 9.25 (s, 1H), 7.50-7.38 (m, 3H), 6.92 (s, 1H), 6.64-6.53 (m, 2H), 5.94 (bs, 2H), 3.51 (d, J=6.1 Hz, 2H), 2.77 (t, J=7.3 Hz, 4H), 2.57 (t, J=7.4 Hz, 4H), 1.99-1.87 (m, 4H). ¹³C NMR (DMSO-d₆): δ 165.71, 152.55, 142.69, 137.23, 129.12, 128.54, 124.82, 117.75, 112.48, 45.14, 32.35, 30.17, 25.01. MS (ESI): m/z calculated for C₂₀H₂₄N₃O₃S [M+H]⁺ 386.49; found, 386.32.

Compound 7b of Formula (III). 88% yield. ¹H NMR (400 MHz, DMSO-d₆): δ 9.22 (s, 1H), 7.47 (d, J=8.1 Hz, 1H), 7.42 (d, J=8.7 Hz, 2H), 6.91 (s, 1H), 6.56 (d, J=8.7 Hz, 2H), 5.89 (s, 2H), 3.87-3.80 (m, 1H), 2.77 (t, J=7.2 Hz, 4H), 2.54-2.51 (m, 4H), 1.97-1.89 (m, 4H), 1.17 (dd, J=7.0, 4.0 Hz, 3H). ¹³C NMR (DMSO-d₆): δ 170.25, 152.98, 143.22, 137.82, 129.63, 128.90, 126.40, 118.28, 112.98, 52.18, 32.88, 30.54, 25.55, 20.37. MS (ESI): m/z calculated for C₂₁H₂₆N₃O₃S [M+H]⁺ 400.52; found, 400.40.

Compound 7c of Formula (III). 98% yield. ¹H NMR (400 MHz, DMSO-d₆): δ 9.25 (s, 1H), 7.46-7.35 (m, 2H), 7.14 (d, J=8.3 Hz, 1H), 6.90 (s, 1H), 6.57-6.47 (m, 2H), 5.85 (s, 2H), 3.62 (t, J=7.1 Hz, 1H), 2.75 (t, J=7.4 Hz, 4H), 2.48-2.44 (m, 4H), 1.94-1.87 (m, 5H), 0.90 (d, J=6.7 Hz, 3H), 0.81 (d, J=6.8 Hz, 3H). ¹³C NMR (DMSO-d₆): δ 168.37, 152.36, 142.62, 137.20, 129.10, 128.35, 126.08, 117.67, 112.29, 61.01, 32.30, 31.22, 30.18, 24.98, 19.22, 17.57. MS (ESI): m/z calculated for C₂₃H₃₀N₃O₃S [M+H]⁺ 428.57; found, 428.29.

Compound 7d of Formula (III). 87% yield. ¹H NMR (400 MHz, DMSO-d₆): δ 9.25 (s, 1H), 7.45-7.32 (m, 2H), 7.13 (d, J=8.9 Hz, 2H), 6.90 (s, 1H), 6.59-6.47 (m, 2H), 3.64-0.360 (m, 2H), 2.75 (t, J=7.3 Hz, 4H), 2.47-2.44 (m, 4H), 1.94-1.87 (m, 5H), 0.90 (d, J=6.7 Hz, 3H), 0.81 (d, J=6.8 Hz, 3H). ¹³C NMR (DMSO-d₆): δ 173.69, 157.59, 147.94, 142.52, 134.41, 133.67, 131.46, 123.00, 117.67, 66.33, 37.62, 36.54, 35.50, 30.30, 24.54, 22.88. MS (ESI): m/z calculated for C₂₃H₃₀N₃O₃S [M+H]⁺ 428.16; found, 428.53.

Compound 7e of Formula (III). 73% yield. 1H NMR (400 MHz, DMSO-d₆): δ9.30 (s, 1H), 7.48-7.38 (m, 3H), 6.92 (s, 1H), 6.60-6.50 (m, 2H), 3.82-3.76 (m, 1H), 2.78 (t, J=7.4 Hz, 4H), 2.57-2.53 (m, 3H), 2.46-2.40 (m, 1H), 2.01-1.85 (m, 4H), 1.72-1.59 (m, 1H), 1.44-1.34 (m, 2H), 0.85 (d, J=6.7 Hz, 3H), 0.76 (d, J=6.5 Hz, 3H). ¹³C NMR (DMSO-d₆): δ 169.62, 152.32, 142.62, 137.32, 129.15, 128.34, 126.14, 117.68, 112.36, 54.55, 42.37, 32.33, 30.04, 25.03, 23.84, 22.83, 21.38. MS (ESI): m/z calculated for C₂₄H₃₂N₃O₃S [M+H]⁺ 442.60; found, 442.41.

Compound 7f of Formula (III). yield 72%. ¹H NMR (400 MHz, DMSO-d₆): δ 9.58 (s, 1H), 8.19-8.02 (m, 1H), 7.47-7.42 (m, 4H), 7.38-7.21 (m, 3H), 6.90 (s, 1H), 6.58-6.48 (m, 2H), 5.88 (s, 2H), 5.11-5.08 (m, 1H), 2.74 (t, J=7.3 Hz, 4H), 2.41-2.25 (m, 4H), 1.95-1.78 (m, 4H). ¹³C NMR (DMSO-d₆): δ 167.23, 152.36, 142.65, 138.40, 137.13, 128.81, 128.30, 127.97, 127.40, 126.88, 126.00, 125.95, 117.76, 112.27, 59.24, 32.22, 29.81, 24.96. MS (ESI): m/z calculated for C₂₆H₂₈N₃O₃S [M+H]⁺ 462.55; found, 462.55.

Compound 7g of Formula (III). 86% yield. ¹H NMR (400 MHz, DMSO-d₆): δ 9.38 (s, 1H), 7.65-7.61 (m, 1H), 7.32-7.20 (m, 7H), 6.90 (s, 1H), 6.48 (d, J=8.7 Hz, 2H), 5.86 (s, 2H), 4.09-4.05 (m, 1H), 2.87-2.83 (m, 2H), 2.78-2.73 (m, 4H), 2.49-2.40 (m, 4H), 1.91-1.87 (m, 4H). ¹³C NMR (DMSO-d₆): δ 168.45, 152.22, 142.55, 137.19, 136.99, 129.24, 128.98, 128.16, 127.86, 126.30, 126.15, 117.61, 112.28, 57.17, 32.29, 29.95, 25.00. MS (ESI): m/z calculated for C₂₇H₃₀N₃O₃S [M+H]⁺ 476.61; found, 476.59.

Compound 7h of Formula (III). 79% yield. ¹H NMR (400 MHz, DMSO-d₆): δ 9.38 (s, 1H), 7.93 (s, 1H), 7.29 (d, J=8.7 Hz, 2H), 7.25-7.16 (m, 5H), 6.89 (s, 1H), 6.47 (d, J=8.7 Hz, 2H), 5.83 (s, 2H), 4.09 (t, J=7.3 Hz, 1H), 2.76-2.72 (m, 5H), 2.45-2.30 (m, 5H), 1.91-1.84 (m, 4H). ¹³C NMR (DMSO-d₆): δ 168.47, 152.21, 142.54, 137.20, 129.24, 128.16, 127.86, 126.14, 117.60, 112.28, 57.21, 32.29, 29.95, 25.01. MS (ESI): m/z calculated for C₂₇H₃₀N₃O₃S [M+H]⁺ 476.61; found, 476.27.

Compound 7k of Formula (III). 77% yield. ¹H NMR (400 MHz, DMSO-d₆): δ 10.15 (s, 1H), 8.24 (d, J=8.7 Hz, 2H), 7.95 (t, J=5.8 Hz, 1H), 7.74 (s, 1H), 7.45-7.41 (m, 2H), 6.74 (s, 2H), 3.76-3.70 (m, 2H), 3.60 (t, J=7.3 Hz, 4H), 3.44 (t, J=7.3 Hz, 4H), 3.25 (t, J=7.4 Hz, 2H), 2.79-2.72 (m, 4H). ¹³C NMR (DMSO-d₆): δ 167.73, 152.38, 142.57, 137.20, 129.54, 128.35, 124.93, 117.56, 112.52, 35.43, 32.33, 30.21, 24.93. MS (ESI): m/z calculated for C₂₁H₂₆N₃O₃S [M+H]⁺ 400.52; found, 400.37.

Compound 10 of Formula (III). 70% yield. ¹H NMR (400 MHz, DMSO-d₆): δ 9.27 (s, 1H), 7.50 (d, J=9.1 Hz, 1H), 7.36-7.28 (m, 2H), 6.98-6.91 (m, 2H), 6.88 (s, 1H), 6.65-6.57 (m, 2H), 6.53-6.45 (m, 2H), 4.04-3.98 (m, 1H), 2.76 (t, J=7.1 Hz, 5H), 2.67-2.52 (m, 2H), 2.45-2.29 (m, 4H), 1.95-1.84 (m, 4H). ¹³C NMR (DMSO-d₆): δ 169.10, 156.37, 152.73, 143.09, 137.77, 130.73, 129.60, 128.78, 127.55, 127.06, 118.14, 115.22, 112.90, 58.04, 32.88, 30.55, 25.60. MS (ESI): m/z calculated for C₂₇H₃₀N₃O₄S [M+H]⁺ 492.61; found, 492.68.

Compound 11 of Formula (III). 91% yield. ¹H NMR (400 MHz, DMSO-d₆): δ 9.29 (s, 1H), 7.64 (s, 3H), 7.42 (d, J=8.7 Hz, 2H), 7.37 (d, J=8.4 Hz, 1H), 6.92 (s, 1H), 6.55 (d, J=8.7 Hz, 2H), 3.85-3.74 (m, 1H), 2.80-2.72 (m, 6H), 2.67-2.62 (m, 1H), 2.48-2.42 (m, 4H), 2.01-1.89 (m, 4H), 1.69-1.21 (m, 5H). ¹³C NMR (DMSO-d₆): δ 168.91, 152.44, 142.66, 137.22, 128.99, 128.32, 127.67, 125.86, 117.77, 112.36, 55.66, 33.05, 32.31, 30.06, 28.91, 26.51, 25.02, 21.96. MS (ESI): m/z calculated for C₂₄H₃₃N₄O₃S [M+H]⁺ 457.61; found, 457.40.

### Example 17: Evaluation of the Efficacy and Selectivity of the Compounds of Formula (III) in Inhibiting the NLRP3-Mediated Release of IL-13 In Vitro

The capability of our compounds of Formula (III) to reduce IL-1β release induced by NLRP3 activation has been investigated in bone marrow derived macrophages (BMDMs) from wild type (WT) mice first primed with 1 μg/ml lipopolysaccharide (LPS) from Escherichia coli for 2 hours. The compounds of Formula (I) have been added at 1 μM of concentration for 30 minutes and then cells have been stimulated with 2′(3′)-O-(4-benzoylbenzoyl)adenosine 5′-triphosphate triethylammonium salt (Bz-ATP) at 100 μM of concentration for 30 minutes. MCC950 (N-((1,2,3,5,6,7-hexahydro-s-indacen-4-yl)carbamoyl)-4-(2-hydroxypropan-2-yl)furan-2-sulfonamide) has been studied as a comparison. Cell culture supernatants were analyzed for IL-1β by ELISA. The potency of the compounds was expressed as percentage of inhibition of the release of IL-1β at 1 μM of concentration and results were reported in Table 1.

TABLE 1
Percentage inhibition of the release of IL-1β in BMDMs from
WT mice following stimulation with LPS + BzATP and treatment
with synthesized compounds of Formula (III) at 1 μM concentration.

| | Inhibitory rate | | Inhibitory |
|---|---|---|---|
| Compd | of IL-1β release | Compd | rate of IL-1β release |
| MCC950 | 60.1 | 7b | 17.3 |
| 6c | 59.4 | 7c | 50.2 |
| 6d | 11.2 | 7d | 8.4 |
| 6e | 53.9 | 7e | 31.5 |
| 6f | 44.8 | 7f | 16.1 |
| 6g | 19.8 | 7g | 38.0 |
| 6h | 49.5 | 7h | 46.4 |
| 8 | 34.7 | 10 | 58.8 |
| 9 | 47.5 | 11 | 28.4 |
| 6k | 36.5 | 7k | 22.3 |

All compounds of Formula (III) were found to be able to inhibit the release of IL-1β, preferably compounds 6c, 6e, 7c, 6 h and 10 of Formula (III).

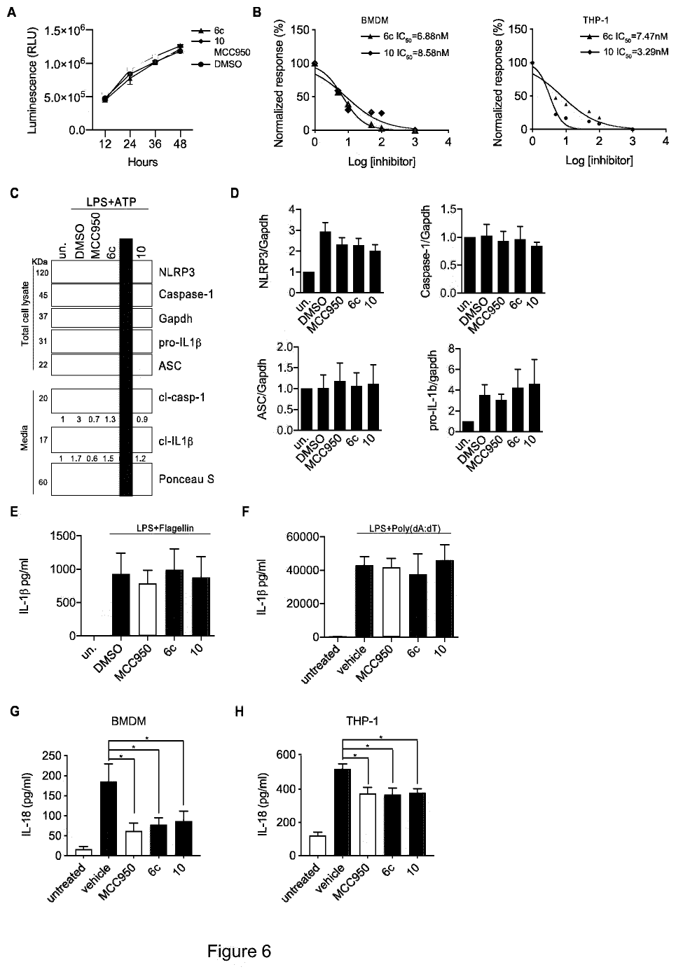

Advantageously, the percentage inhibition of the release of IL-1β of 6c and 10 compounds of Formula (III) was excellent: 59.4% for compound 6c and 58.8% for compound 10. Compounds 6c and 10 thus demonstrated a potency comparable to that of MCC950 (60.1%), one of the most potent NLRP3 inhibitors known to date whose clinical development has been discontinued due to hepatotoxicity problems. The IC₅₀ values obtained in BMDM stimulated with LPS, compounds and ATP were 6.88 nm for compound 6c of Formula (III) and 8.58 nm for compound 10 of Formula (III) (FIG. 6B). The potency of compounds 6c and 10 of Formula (III) was also evaluated in human monocytes (THP-1) stimulated with LPS, compounds and ATP: IC₅₀ values obtained in THP-1 cells were 7.47 nm for compound 6c of Formula (III) and 3.29 nm for compound 10 (FIG. 6B). The compounds of the invention of Formula (III) thus identify a new chemotype of NLRP3 inhibitors that could have the advantage of a better toxicity profile than MCC950 in future clinical trials.

The selectivity of compounds 6c and 10 of Formula (III) as inhibitors of NLRP3 was then evaluated with respect to NLRC4 inflammasome. For activation of NLRC4 inflammasome, BMDM were treated with LPS (100 ng/ml) for 3 hours. Subsequently the culture medium was removed and replaced with a serum-free medium containing compound 4 of Formula (III) (1 μm for 30 minutes), finally the cells were transfected with flagellin (100 ng/ml) by S. typhimurium for 2 hours. The supernatant was tested by ELISA. Compounds had no effect on the activation of NLRC4 inflammasome triggered by flagellin S. typhimurium (FIG. 6E), demonstrating specificity in the inhibition of NLRP3 inflammasome.

### Example 18: Evaluation of the Effect of Compounds 6c and 10 of Formula (III) on Cell Viability of THP-1 Cells

Cell viability was evaluated on THP-1 cells using realtime-Glo” MT Cell Viability Assay (Promega Italia, MI). Briefly, the cells were seeded in a 96 multiwell (10×10⁴ cells/well), treated with the selected compound, and incubated for 48 hours. After treatment, the cells were incubated for 10 minutes in the incubator with the realtime-Glo” reagent following protocol. The luminescence was measured every 12 hours in the Glomax Multi Detection System Promega. The luminescence signal correlates with the number of metabolically active cells. The results represent the mean luminescence values (RLU)±SEM. Compounds 6c and 10 of Formula (III), at the concentration used, show no cytotoxicity and therefore does not affect cell viability of THP-1 cells (FIG. 6A).

### Example 19: Evaluation of the Effect of Compounds 6c and 10 on the Protein Expression of NLRP3 Inflammasome

Total cell lysates of THP-1 cells were prepared in RIPA buffer (50 mM Tris-HCl pH 7.8, 150 mM NaCl, 1% IGEPAL CA-630, 0.5% sodium deoxycholate, 0.1% SDS, 1 mM dithiothreitol (DTT)) supplemented with proteases and phosphatases inhibitors. Supernatant was concentrated using Pierce Protein Concentrators PES 10K MWCO (ThermoFisher) then was centrifuged at 4000×g for 15 minutes. A total of 20 μg of protein or a total of 10 μl of concentrated medium was separated by SDS-PAGE and transferred to nitrocellulose membranes for standard western blotting. The following primary antibodies were used: NLRP3 (Adipogen, #AG-20B-0014-C100), ASC (Adipogen, #AG-20B-0014-C100) Caspasi1 (Novus Biological, #14F468) and IL-1β (Cell Signaling Technology, #12242), GAPDH (Cell Signaling Technology, #2118). Isotype-matched horseradish peroxidase-conjugated secondary antibodies were used, followed by detection using chemiluminescence (GE Image-Quant).

Treatment with these compounds did not affect the priming phase of activation of NLRP3 inflammasome, not altering the expression of NLRP3, caspase-1, ASC and pro-IL-1β in total cell lysate (FIG. 6C-D). The compounds significantly reduced the release of IL-1β and the amount of caspase-1 cleaved into the cellular supernatant (FIG. 6C).

FIGS. 6G and 6H report the results for NLRP3-stimulated secretion of IL-18 in BMDM and THP-1 cells, respectively. Data are presented as mean±SEM from three independent experiments. * p<0.05.

### Example 20: Evaluation of the Effect of Compounds 6c and 10 of Formula (III) on LPS-Induced Inflammation In Vivo and Ex Vivo

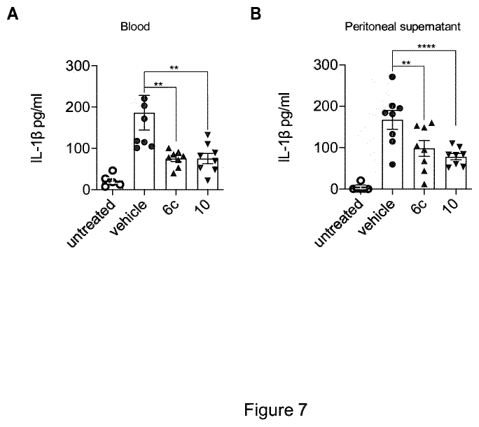

C57BL/6 mice were treated with inhibitors at a concentration of 25 mg/Kg or vehicle (DMSO), administered by intraperitoneal injection for 30 minutes. The mice were subsequently treated with LPS 1 mg/Kg for 4 hours, administered intraperitoneally. Mice were sacrificed and blood and peritoneal supernatant were collected. The blood was centrifuged (1000×g for 15 minutes) to obtain the plasma. ELISA tests were carried out on plasma and peritoneal supernatant to assess IL-1β levels. Mice receiving treatment with compounds showed a significant reduction in IL-1β release compared to mice treated with the vehicle, indicating that the compound had a strong efficacy in reducing the activation of NLRP3 inflammasome also in vivo (FIG. 7).

### Example 21: Evaluation of the Effect of Compounds 6c and 10 of Formula (III) on Tumor Growth In Vivo

Procedures involving animals and their care were in conformity with institutional guidelines, and the Animal Ethics Committee approved all experimental protocols (Authorization N^{o} 481/2017-PR and CBCC2.N.BH4 approved by Italian Ministry of Health). A total of 1×10⁶ B16-F10 melanoma cells transfected with cytluc (B16-F10cytLUC) were subcutaneously inoculated into females, 6- to 8-week-old C₅₇BL/6 mice. Tumor growth was monitored daily, and tumor volumes were measured every other day with calipers using the following equation: Volume=π/6×(a×b2), where “a” is the major diameter and “b” is the minor diameter. As soon as the mass was palpable, mice were randomly divided (5 mice per group) into the treatment groups and the control group, with 5 mice per group. For NLRP3 inflammasome inhibition, mice were intraperitoneal injected with selected molecules thrice weekly at 25 mg/Kg; control mice received equal volumes of DMSO.

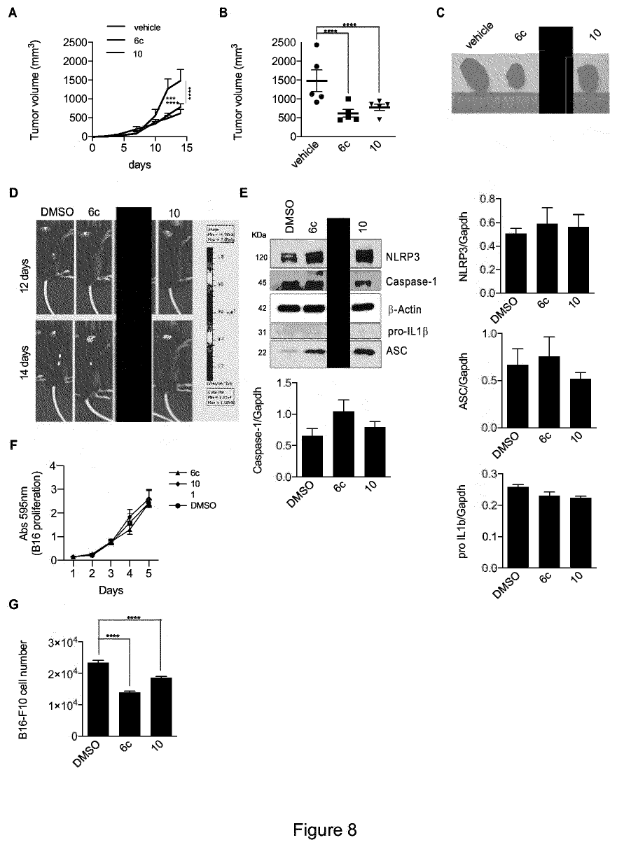

Luciferase luminescence was followed by a total body luminometer (IVIS Lumina, Caliper-PerkinElmer). Briefly, mice were anesthetized with 2.5% isoflurane intraperitoneal injected with 150 mg/Kg d-luciferin (Promega) and luminescence quantified after 15 min using the Living Image Software (Caliper). Mice treated with compounds developed a mass that was significantly smaller (about 50% reduction) than mice treated with the vehicle (DMSO) (FIG. 8A-D). In the lysates of the extracted tumor masses there was no alteration in the expression of the proteins NLRP3, caspase-1, ASC and pro-IL-1β (FIG. 8E).

### Example 22: B16-F10 Cancer Cells Proliferation after Treatment with Compounds 6c and 10 of Formula (III)

B16-F10 cells were treated with selected inhibitors (1 μM) then were counted with a Burker chamber and plated in five sets of 4 wells of a 24-well plate. Starting from the following day (day 1) 1 set of wells (at days 2, 3, 4 and 5) was washed once with PBS, fixed in 4% formaldehyde solution for 15 min at room temperature, and then kept in PBS at 4° C. At day 5, all the wells were stained with crystal violet for 20 minutes, lysed with 10% acetic acid, then the absorbance was read at 595 nm. Compounds had no direct effect on cell proliferation of B16-F10 melanoma cells (FIG. 8F).

### Example 23: Evaluation of the Effect of Compounds 6c and 10 of Formula (III) on the Tumor Microenvironment by Co-Culture Model

Co-culture of peritoneal macrophages and B16-F10 cells were performed using Transwell chambers with 0.4-μm pores on the membranes (Corning, Corning, NY, USA). Peritoneal macrophages and B16-F10 cells were cultured in the lower and upper compartments of the Transwell chamber, respectively, for 48 h. Peritoneal macrophages were seeded at a density of 2×10⁵ cells and treated with LPS (1 μg/mL) for 2 hours, then DMSO or compounds of Formula (III) (1 μM) for 30 minutes and finally stimulated with ATP for 1 hour. B16-F10 cells were seeded at a density of 2×10³ cells each well. After 48 h, B16-F10 cells in the upper chambers were removed and counted using an automated cell counter (Tali™ image-based cytometer (Invitrogen). B16-F10 cells in contact with peritoneal macrophages treated with the vehicle alone grew more than B16-F10 cells in contact with peritoneal macrophages treated with compounds (FIG. 8G). Compounds had an effect on the tumor microenvironment, reducing the activation of NLRP3 inflammasome.

### Preparation and Evaluation of Compounds of Formula (IV)

The compounds of Formula (IV) of the invention were prepared according to the scheme shown in FIG. 9.

### Example 24: Preparation of Compounds 2a-b of Formula (IV)

To a solution of 1a-b (1.0 mmol) in water (5 mL) sodium hydrogen carbonate (NaHCO₃, 2.5 mmol) was added under vigorous stirring. Then 4-nitrobenzenesulfonyl chloride (1.0 mmol) was added in small portions over 1 h and the reaction was stirred at room temperature for 16 h. Next, the reaction mixture was acidified to pH 2 using 1 M HCl and the aqueous phase was extracted with ethyl acetate (3×15 mL). The combined organic layers were washed with brine (1×10 mL), dried over Na₂SO₄ and the solvent was evaporated under reduced pressure. The residual crude was crystallized to yield the desired products.

((4-nitrophenyl)sulfonyl)proline (2a) of Formula (IV). 65% yield. ¹H NMR (400 MHz, DMSO-d₆): δ 12.84 (bs, 1H), 8.42-8.36 (m, 2H), 8.11-8.06 (m, 2H), 4.20 (dd, J=8.6, 3.8 Hz, 1H), 3.44-3.35 (m, 1H), 3.25-3.21 (m, 1H), 2.02-1.94 (m, 1H), 1.91-1.75 (m, 2H), 1.71-1.60 (m, 1H). 1-((4-nitrophenyl)sulfonyl)piperidine-2-carboxylic acid (2b) of Formula (IV). 55% yield. ¹H NMR (400 MHz, DMSO-d₆): δ 12.97 (bs, 1H), 8.42-8.33 (m, 2H), 8.08-8.00 (m, 2H), 4.62 (d, J=5.2 Hz, 1H), 3.75-3.71 (m, 1H), 3.20-3.09 (m, 1H), 2.07-2.00 (m, 1H), 1.62-1.58 (m, 3H), 1.30-1.18 (m, 2H).

### Example 25: Preparation of Compounds 3a-b of Formula (IV)

To an ice-cooled solution of 2a-b (1.1 mmol) in DMF (5 mL) HATU (1.1 mmol) and DIPEA (1.1 mmol) were added. Then, a solution of hexahydro-s-indacen-4-amine (1.0 mmol) in DMF (2 mL) was added dropwise. The resulting mixture was warmed to room temperature and left stirring for 2-5 h. After the removal of the solvent, the crude was dissolved with ethyl acetate (20 mL) and the organic layer was sequentially washed with 10% aqueous solution of citric acid (1×10 mL), 5% aqueous solution of NaHCO₃ (1×10 mL) and brine (1×10 mL). After drying over Na₂SO₄, the solvent was evaporated to yield a solid residue which was firstly triturated with diethyl ether, then filtered and the solid was re-crystallized from methanol to give the desired derivatives.

Compound 3a of Formula (IV). 88% yield. ¹H NMR (400 MHz, DMSO-d₆): δ9.48 (s, 1H), 8.42 (d, J=8.9 Hz, 2H), 8.19-8.07 (m, 2H), 6.95 (s, 1H), 4.26-4.23 (m, 1H), 3.61-3.49 (m, 1H), 2.80 (t, J=7.3 Hz, 4H), 2.66 (t, J=7.4 Hz, 4H), 2.03-1.84 (m, 8H), 1.72-1.59 (m, 1H). ¹³C NMR (DMSO-d₆): δ 168.97, 149.82, 142.67, 137.61, 129.63, 129.03, 127.93, 125.40, 123.65, 118.74, 117.19, 62.16, 60.71, 32.35, 31.32, 29.97, 26.23, 24.94. MS (ESI): m/z calculated for C₂₃H₂₆N₃O₅S [M+H]⁺ 456.54; found, 456.41.

Compound 3b of Formula (IV). 77% yield. ¹H NMR (400 MHz, DMSO-d₆): δ9.42 (s, 1H), 8.51-8.25 (m, 2H), 8.12-7.86 (m, 2H), 6.92 (s, 1H), 4.76-4.72 (m, 1H), 3.84-3.80 (m, 1H), 3.66-3.55 (m, 1H), 2.78 (t, J=7.3 Hz, 4H), 2.60-2.53 (m, 4H), 2.14-2.10 (m, 1H), 2.01-1.85 (m, 4H), 1.74-1.62 (m, 3H), 1.40-1.33 (m, 2H). ¹³C NMR (DMSO-d₆): δ 167.65, 149.36, 145.24, 142.73, 137.28, 128.87, 128.21, 124.30, 117.93, 54.40, 42.87, 32.27, 30.10, 28.85, 24.83, 24.24, 18.95. MS (ESI): m/z calculated for C₂₄H₂₈N₃O₅S [M+H]⁺ 470.56; found, 470.24.

### Example 26: Preparation of Compounds 4a-b of Formula (IV)

The nitro derivatives 3a-b (1 mmol) were dissolved in ethyl acetate (15 mL) and glacial CH₃COOH (0.5 mL). A catalytic amount (0.1 mmol) of palladium on activated charcoal (10% Pd basis) was added under hydrogen atmosphere. After 16 h, the reaction mixture was filtered through Celite. The filtrate was concentrated under reduced pressure and the crudes were purified via semi-preparative HPLC to give compounds 4a-b of Formula (IV).

Compound 4a of Formula (IV). 84% yield. 1H NMR (400 MHz, DMSO-d₆): δ 9.25 (s, 1H), 7.49 (d, J=8.7 Hz, 2H), 6.95 (s, 1H), 6.64 (d, J=8.8 Hz, 2H), 6.05 (s, 2H), 4.02-3.99 (m, 1H), 3.47-3.39 (m, 1H), 3.14-3.04 (m, 1H), 2.80 (t, J=7.2 Hz, 4H), 2.67 (t, J=7.3 Hz, 4H), 2.01-1.90 (m, 4H), 1.88-1.74 (m, 3H), 1.58-1.48 (m, 1H). ¹³C NMR (DMSO-d₆): δ 170.15, 153.69, 143.20, 138.21, 129.86, 121.74, 118.39, 113.25, 62.16, 49.62, 32.93, 31.64, 30.55, 25.55, 24.71. MS (ESI): m/z calculated for C₂₃H₂₈N₃O₃S [M+H]⁺ 426.55; found, 426.29.

Compound 4b of Formula (IV). Yield 71%. ¹H NMR (400 MHz, DMSO-d₆): δ 9.25 (s, 1H), 7.48-7.32 (m, 2H), 6.93 (s, 1H), 6.61-6.48 (m, 2H), 4.54 (d, J=4.1 Hz, 1H), 3.64-3.56 (m, 1H), 3.47 (td, J=12.7, 2.5 Hz, 1H), 2.79 (t, J=7.3 Hz, 4H), 2.69-2.52 (m, 4H), 2.04-1.89 (m, 5H), 1.61-1.47 (m, 3H), 1.45-1.18 (m, 2H). ¹³C NMR (DMSO-d₆): δ 168.90, 153.03, 143.26, 137.88, 129.80, 129.16, 125.31, 118.32, 113.16, 54.69, 42.71, 32.91, 30.70, 28.33, 25.51, 24.37, 19.74. MS (ESI): m/z calculated for C₂₄H₃₀N₃O₃S [M+H]⁺ 440.58; found, 440.37.

### Example 27: Evaluation of the Efficacy of the Compounds of Formula (IV) in Inhibiting the NLRP3-Mediated Release of IL-113 In Vitro

The capability of our compounds to reduce IL-1β release induced by NLRP3 activation has been investigated in bone marrow derived macrophages (BMDMs) from wild type (WT) mice first primed with 1 μg/ml lipopolysaccharide (LPS) from Escherichia coli for 2 hours. The compounds of Formula (IV) have been added at 1 μM of concentration for 30 minutes and then cells have been stimulated with 2′(3′)-O— (4-benzoylbenzoyl)adenosine 5′-triphosphate triethylammonium salt (Bz-ATP) at 100 μM of concentration for 30 minutes. Cell culture supernatants were analyzed for IL-1β by enzyme-linked immunosorbent assay (ELISA).

It has also been used as a comparison MCC950 (N-((1,2,3,5,6,7-hexahydro-s-indacen-4-yl)carbamoyl)-4-(2-hydroxypropan-2-yl)furan-2-sulfonamide). The potency of the compounds was expressed as percentage of inhibition of the release of IL-1β at the concentration of 1 μM (Table 2).

TABLE 2
Percentage inhibition of the release of IL-1β in BMDMs
from WT mice following stimulation with LPS + BzATP
and treatment with synthesized compounds at 1 μm concentration.

| Compd of | Inhibitory rate | Compd of | Inhibitory rate |
|---|---|---|---|
| Formula (IV) | of IL-1β release | Formula (IV) | of IL-1β release |
| MCC950 | 60.1 | | |
| 3a | 25.7 | 4a | 41.6 |
| 3b | 52.4 | 4b | 25.4 |

The compounds of Formula (IV) have been shown to inhibit the release of IL-1 with variable potency. Among these, compound 3b was the most potent with a percentage of inhibition of 52.4%, comparable to that of MCC950 (60.1%), one of the most potent NLRP3 inhibitors known to date whose clinical development has been interrupted because of hepatotoxicity problems. The potency of compound 4a of Formula (IV) (41.6%) was slightly lower. The compounds of Formula (IV) thus identify a new chemotype of NLRP3 inhibitors that could have the advantage of a better toxicity profile than MCC950 in future clinical trials.

1. An hexahydro-s-indacene compound of Formula (I) or its pharmaceutically acceptable salt: 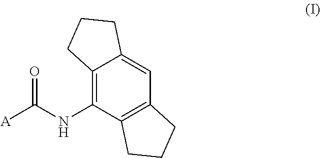
wherein A is selected from the group consisting of: 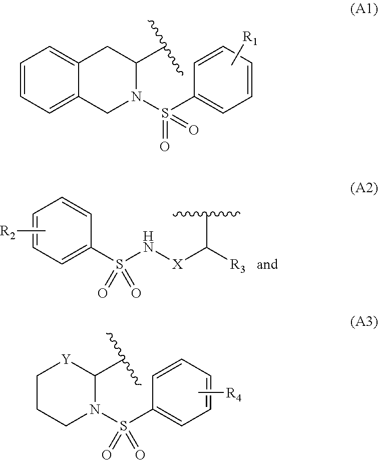
where
R₁ is a substituent selected from H, halogen, CF₃, (C₁-C₃)alkyl, (C₁-C₃)alkoxy, NH₂, NO₂, CN, COOH, a heterocyclic substituent selected from the group consisting of piperidine, morpholine and optionally substituted piperazine, —NHCH₂Ph, —N((C₁-C₂)alkyl)₂, —NH((C₂-C₄)alkyl)-NH₂, COO((C₁-C₂)alkyl and —NHEt;
X is —(CH₂)ₙ— and n is equal to 0 o 1;
R₂ is a substituent selected from NH₂ and NO₂;
R₃ is a substituent selected from the group consisting of H, alkyl(C₁-C₄) alkyl(C₁-C₄)NH₂, phenyl, benzyl and hydroxybenzyl;
Y is —(CH₂)ₙ— and n is equal to 0 or 1; and
R₄ is a substituent selected from NH₂ and NO₂.

2. The hexahydro-s-indacene compound of claim 1, wherein A is A1 and the compound of Formula (I) is a tetrahydroisoquinoline compound or its pharmaceutically acceptable salt of formula (II): 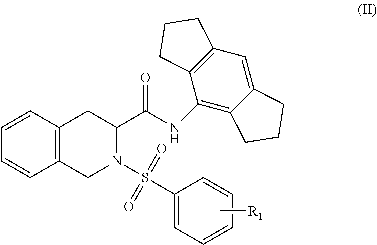
wherein
R₁ is a substituent selected from H, halogen, CF₃, (C₁-C₃)alkyl, (C₁-C₃)alkoxy, NH₂, NO₂, CN, COOH, a heterocyclic substituent selected from the group consisting of piperidine, morpholine and optionally substituted piperazine, —NHCH₂Ph, —N((C₁-C₂)alkyl)₂, —NH((C₂-C₄)alkyl)-NH₂, COO(C₁-C₂)alkyl and —NHEt.

3. The hexahydro-s-indacene compound of claim 1, wherein R₁ is NH₂ or NO₂.

4. The hexahydro-s-indacene compound of claim 1, wherein when R₁ is (C₁-C₃)alkyl, it is methyl, ethyl, propyl or isopropyl.

5. The hexahydro-s-indacene compound of claim 1, wherein when R₁ is halogen, it is fluorine, chlorine, bromine or iodine, preferably fluorine.

6. The hexahydro-s-indacene compound of claim 1, wherein when R₁ is (C₁-C₃)alkoxy, it is methoxy, ethoxy, propoxy or isopropoxy.

7. The hexahydro-s-indacene compound of claim 1, wherein when R₁ is a heterocyclic substituent selected from the group consisting of piperidine, morpholine and optionally substituted piperazine, it is piperidine, morpholine or piperazine, the latter being optionally substituted on the nitrogen atom with, preferably, methyl or tert-butoxycarbonyl.

8-11. (canceled)

12. The hexahydro-s-indacene compound of claim 1, wherein the compound of Formula (I) is a tetrahydroisoquinoline compound of Formula (II) selected from the group consisting of:
(S)—N-(1,2,3,5,6,7-hexahydro-s-indacen-4-yl)-2-((4-nitrophenyl)sulfonyl)-1,2,3,4-tetrahydroisoquinoline-3-carboxamide (3 of Formula (II)) 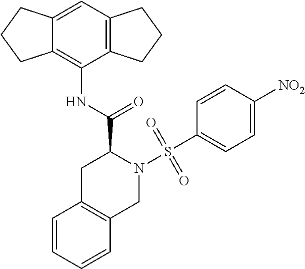 [SMILES: O=C(Nc1c2c(cc3c1CCC3)CCC2)[C@@H]1Cc2ccccc2CN1S(=O)(=O)c1ccc([N+](=O)[O-])cc1]
(S)-2-((4-aminophenyl)sulfonyl)-N-(1,2,3,5,6,7-hexahydro-s-indacen-4-yl)-1,2,3,4-tetrahydroisoquinoline-3-carboxamide (4 of Formula (II)) 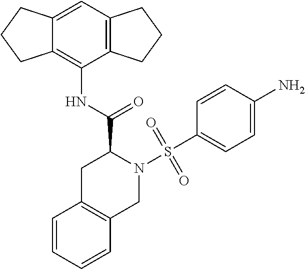 [SMILES: Nc1ccc(S(=O)(=O)N2Cc3ccccc3C[C@H]2C(=O)Nc2c3c(cc4c2CCC4)CCC3)cc1]
(S)-2-((4-fluorophenyl)sulfonyl)-N-(1,2,3,5,6,7-hexahydro-s-indacen-4-yl)-1,2,3,4-tetrahydroisoquinoline-3-carboxamide (6 of Formula (II)) 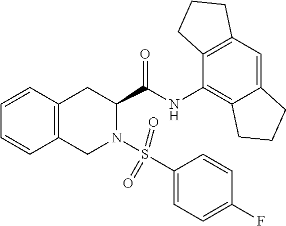 [SMILES: O=C(Nc1c2c(cc3c1CCC3)CCC2)[C@@H]1Cc2ccccc2CN1S(=O)(=O)c1ccc(F)cc1]
(S)-2-((4-(benzylamino)phenyl)sulfonyl)-N-(1,2,3,5,6,7-hexahydro-s-indacen-4-yl)-1,2,3,4-tetrahydroisoquinoline-3-carboxamide (7a of Formula (II)) 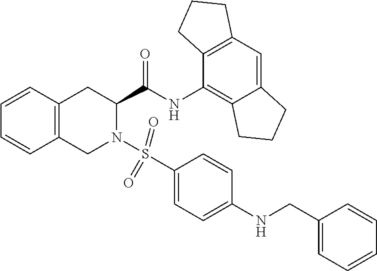 [SMILES: O=C(Nc1c2c(cc3c1CCC3)CCC2)[C@@H]1Cc2ccccc2CN1S(=O)(=O)c1ccc(NCc2ccccc2)cc1]
(S)-2-((4-(dimethylamino)phenyl)sulfonyl)-N-(1,2,3,5,6,7-hexahydro-s-indacen-4-yl)-1,2,3,4-tetrahydroisoquinoline-3-carboxamide (7b of Formula (II)) 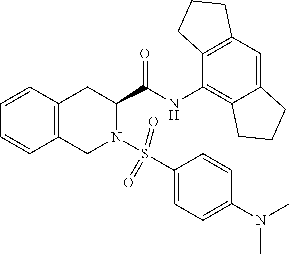 [SMILES: CN(C)c1ccc(S(=O)(=O)N2Cc3ccccc3C[C@H]2C(=O)Nc2c3c(cc4c2CCC4)CCC3)cc1]
(S)—N-(1,2,3,5,6,7-hexahydro-s-indacen-4-yl)-2-((4-(piperidin-1-yl)phenyl) sulfonyl)-1,2,3,4-tetrahydroisoquinoline-3-carboxamide (7c of Formula (II)) 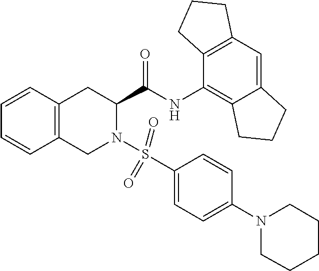 [SMILES: O=C(Nc1c2c(cc3c1CCC3)CCC2)[C@@H]1Cc2ccccc2CN1S(=O)(=O)c1ccc(N2CCCCC2)cc1]
(S)—N-(1,2,3,5,6,7-hexahydro-s-indacen-4-yl)-2-((4-morpholinophenyl)sulfonyl)-1,2,3,4-tetrahydroisoquinoline-3-carboxamide (7 d of Formula (II)) 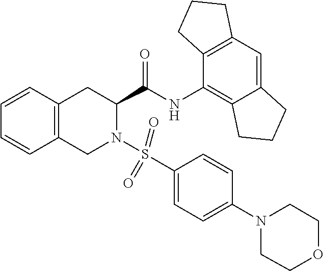 [SMILES: O=C(Nc1c2c(cc3c1CCC3)CCC2)[C@@H]1Cc2ccccc2CN1S(=O)(=O)c1ccc(N2CCOCC2)cc1]
(S)-2-((4-(diethylamino)phenyl)sulfonyl)-N-(1,2,3,5,6,7-hexahydro-s-indacen-4-yl)-1,2,3,4-tetrahydroisoquinoline-3-carboxamide (7e of Formula (II)) 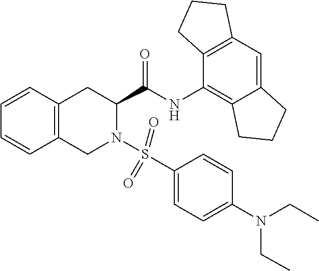 [SMILES: CCN(CC)c1ccc(S(=O)(=O)N2Cc3ccccc3C[C@H]2C(=O)Nc2c3c(cc4c2CCC4)CCC3)cc1]
(S)—N-(1,2,3,5,6,7-hexahydro-s-indacen-4-yl)-2-((4-(piperazin-1-yl)phenyl) sulfonyl)-1,2,3,4-tetrahydroisoquinoline-3-carboxamide (7f of Formula (II)) 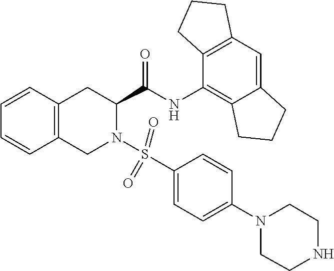 [SMILES: O=C(Nc1c2c(cc3c1CCC3)CCC2)[C@@H]1Cc2ccccc2CN1S(=O)(=O)c1ccc(N2CCNCC2)cc1]
(S)—N-(1,2,3,5,6,7-hexahydro-s-indacen-4-yl)-2-((4-(4-methylpiperazin-1-yl) phenyl)sulfonyl)-1,2,3,4-tetrahydroisoquinoline-3-carboxamide (7g of Formula (II)) 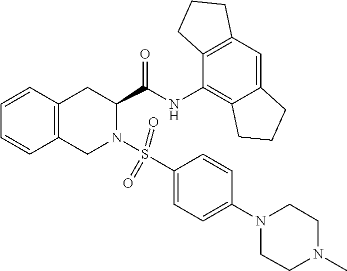 [SMILES: CN1CCN(c2ccc(S(=O)(=O)N3Cc4ccccc4C[C@H]3C(=O)Nc3c4c(cc5c3CCC5)CCC4)cc2)CC1]
tert-butyl(S)-4-(4-((3-((1,2,3,5,6,7-hexahydro-s-indacen-4-yl)carbamoyl)-3,4-dihydroisoquinolin-2(1H)-yl)sulfonyl)phenyl)piperazine-1-carboxylate (7h of Formula (II)) 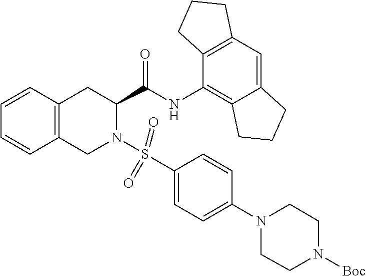 [SMILES: CC(C)(C)OC(=O)N1CCN(c2ccc(S(=O)(=O)N3Cc4ccccc4C[C@H]3C(=O)Nc3c4c(cc5c3CCC5)CCC4)cc2)CC1]
(S)-2-((4-(ethylamino)phenyl)sulfonyl)-N-(1,2,3,5,6,7-hexahydro-s-indacen-4-yl)-1,2,3,4-tetrahydroisoquinoline-3-carboxamide (7i of Formula (II)) 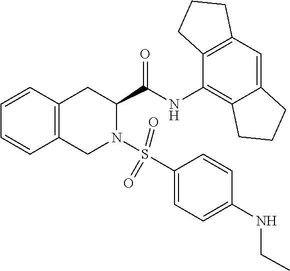 [SMILES: CCNc1ccc(S(=O)(=O)N2Cc3ccccc3C[C@H]2C(=O)Nc2c3c(cc4c2CCC4)CCC3)cc1]
(S)-2-((4-((2-aminoethyl)amino)phenyl)sulfonyl)-N-(1,2,3,5,6,7-hexahydro-s-indacen-4-yl)-1,2,3,4-tetrahydroisoquinoline-3-carboxamide (7j of Formula (II)) 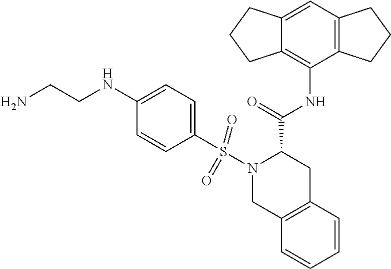 [SMILES: NCCNc1ccc(S(=O)(=O)N2Cc3ccccc3C[C@H]2C(=O)Nc2c3c(cc4c2CCC4)CCC3)cc1]
(S)-2-((4-((4-aminobutyl)amino)phenyl)sulfonyl)-N-(1,2,3,5,6,7-hexahydro-s-indacen-4-yl)-1,2,3,4-tetrahydroisoquinoline-3-carboxamide (7k of Formula (II)) 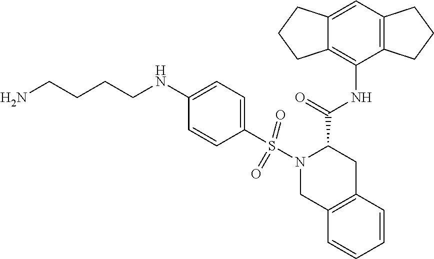 [SMILES: NCCCCNc1ccc(S(=O)(=O)N2Cc3ccccc3C[C@H]2C(=O)Nc2c3c(cc4c2CCC4)CCC3)cc1]
(S)—N-(1,2,3,5,6,7-hexahydro-s-indacen-4-yl)-2-((4-(trifluoromethyl)phenyl)sulfonyl)-1,2,3,4-tetrahydroisoquinoline-3-carboxamide (9a of Formula (II)) 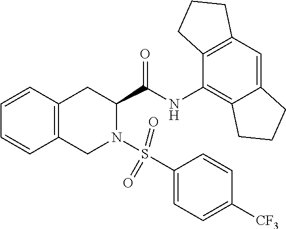 [SMILES: O=C(Nc1c2c(cc3c1CCC3)CCC2)[C@@H]1Cc2ccccc2CN1S(=O)(=O)c1ccc(C(F)(F)F)cc1]
(S)—N-(1,2,3,5,6,7-hexahydro-s-indacen-4-yl)-2-((4-methoxyphenyl)sulfonyl)-1,2,3,4-tetrahydroisoquinoline-3-carboxamide (9b of Formula (II)) 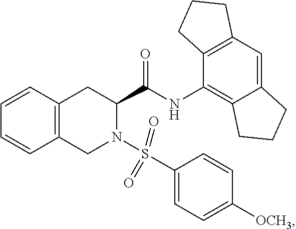 [SMILES: COc1ccc(S(=O)(=O)N2Cc3ccccc3C[C@H]2C(=O)Nc2c3c(cc4c2CCC4)CCC3)cc1]
(S)-2-((4-cyanophenyl)sulfonyl)-N-(1,2,3,5,6,7-hexahydro-s-indacen-4-yl)-1,2,3,4-tetrahydroisoquinoline-3-carboxamide (9c of Formula (II)) 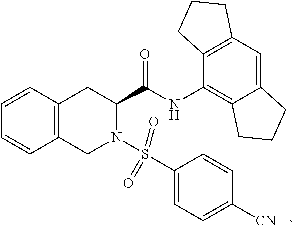 [SMILES: N#Cc1ccc(S(=O)(=O)N2Cc3ccccc3C[C@H]2C(=O)Nc2c3c(cc4c2CCC4)CCC3)cc1]
(S)-Methyl 4-((3-((1,2,3,5,6,7-hexahydro-s-indacen-4-yl)carbamoyl)-3,4-dihydroisoquinolin-2(1H)-yl)sulfonyl)benzoate (9 d of Formula (II)) 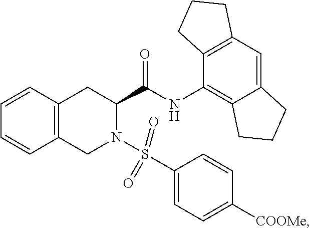 [SMILES: COC(=O)c1ccc(S(=O)(=O)N2Cc3ccccc3C[C@H]2C(=O)Nc2c3c(cc4c2CCC4)CCC3)cc1]
(S)-4-((3-((1,2,3,5,6,7-hexahydro-s-indacen-4-yl)carbamoyl)-3,4-dihydroisoquinolin-2(1H)-yl)sulfonyl)benzoic acid (10 of Formula (II))

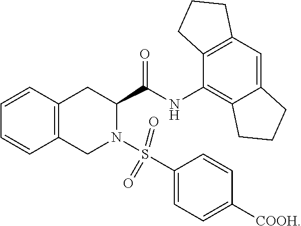 [SMILES: O=C(O)c1ccc(S(=O)(=O)N2Cc3ccccc3C[C@H]2C(=O)Nc2c3c(cc4c2CCC4)CCC3)cc1]

13. The hexahydro-s-indacene compound of claim 1, wherein A is A2 and the compound of Formula (I) is a sulfonamide compound or its pharmaceutically acceptable salt of formula (III): 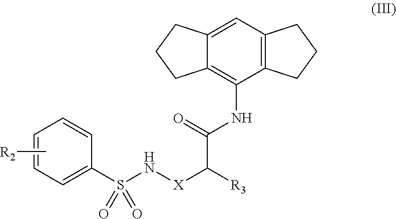
wherein
X is —(CH₂)ₙ— and n is equal to 0 o 1;
R₂ is a substituent selected from NH₂ and NO₂;
R₃ is a substituent selected from the group consisting of H, alkyl(C₁-C₄) alkyl(C₁-C₄)NH₂, phenyl, benzyl and hydroxybenzyl.

14. The hexahydro-s-indacene compound of claim 1, wherein X is —(CH₂)ₙ— and n is equal to 0.

15-17. (canceled)

18. The hexahydro-s-indacene compound of claim 1 wherein the compound of Formula (I) is a sulfonamide compound of formula (III) selected from the group consisting of:
N-(1,2,3,5,6,7-hexahydro-s-indacen-4-yl)-2-((4-nitrophenyl)sulfonamido)acetamide (6a of Formula (III)) 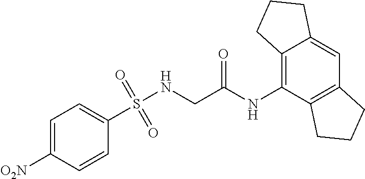 [SMILES: O=C(CNS(=O)(=O)c1ccc([N+](=O)[O-])cc1)Nc1c2c(cc3c1CCC3)CCC2]
(S)—N-(1,2,3,5,6,7-hexahydro-s-indacen-4-yl)-2-((4-nitrophenyl)sulfonamido)propanamide (6b of Formula (III)) 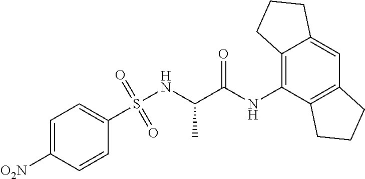 [SMILES: C[C@H](NS(=O)(=O)c1ccc([N+](=O)[O-])cc1)C(=O)Nc1c2c(cc3c1CCC3)CCC2]
(S)—N-(1,2,3,5,6,7-hexahydro-s-indacen-4-yl)-3-methyl-2-((4-nitrophenyl)sulfonamido)butanamide (6c of Formula (III)) 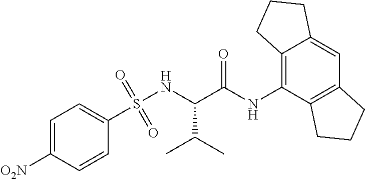 [SMILES: CC(C)[C@H](NS(=O)(=O)c1ccc([N+](=O)[O-])cc1)C(=O)Nc1c2c(cc3c1CCC3)CCC2]
(R)—N-(1,2,3,5,6,7-hexahydro-s-indacen-4-yl)-3-methyl-2-((4-nitrophenyl)sulfonamido)butanamide (6d of Formula (III)) 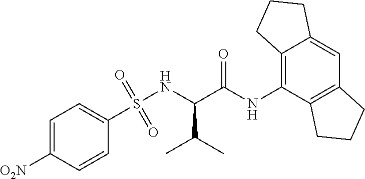 [SMILES: CC(C)[C@@H](NS(=O)(=O)c1ccc([N+](=O)[O-])cc1)C(=O)Nc1c2c(cc3c1CCC3)CCC2]
(S)—N-(1,2,3,5,6,7-hexahydro-s-indacen-4-yl)-4-methyl-2-((4-nitrophenyl)sulfonamido)pentanamide (6e of Formula (III)) 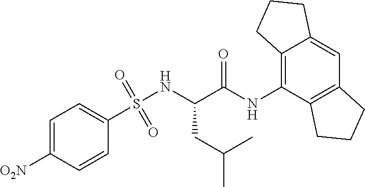 [SMILES: CC(C)C[C@H](NS(=O)(=O)c1ccc([N+](=O)[O-])cc1)C(=O)Nc1c2c(cc3c1CCC3)CCC2]
(S)—N-(1,2,3,5,6,7-hexahydro-s-indacen-4-yl)-2-((4-nitrophenyl)sulfonamido)-2-phenylacetamide (6f of Formula (III)) 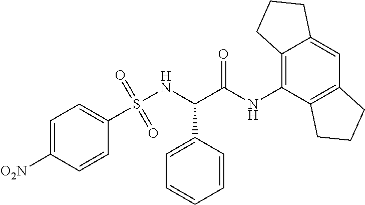 [SMILES: O=C(Nc1c2c(cc3c1CCC3)CCC2)[C@@H](NS(=O)(=O)c1ccc([N+](=O)[O-])cc1)c1ccccc1]
(S)—N-(1,2,3,5,6,7-hexahydro-s-indacen-4-yl)-2-((4-nitrophenyl)sulfonamido)-3-phenylpropanamide (6g of Formula (III)) 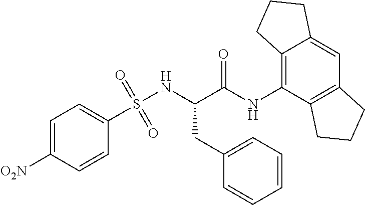 [SMILES: O=C(Nc1c2c(cc3c1CCC3)CCC2)[C@H](Cc1ccccc1)NS(=O)(=O)c1ccc([N+](=O)[O-])cc1]
(R)—N-(1,2,3,5,6,7-hexahydro-s-indacen-4-yl)-2-((4-nitrophenyl)sulfonamido)-3-phenylpropanamide (6h of Formula (III)) 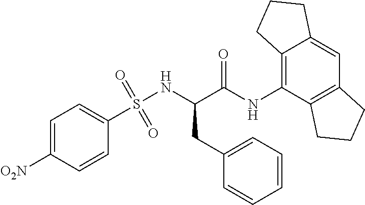 [SMILES: O=C(Nc1c2c(cc3c1CCC3)CCC2)[C@@H](Cc1ccccc1)NS(=O)(=O)c1ccc([N+](=O)[O-])cc1]
(S)—N-(1,2,3,5,6,7-hexahydro-s-indacen-4-yl)-3-(4-hydroxyphenyl)-2-((4-nitrophenyl)sulfonamido)propanamide (8 of Formula (III)) 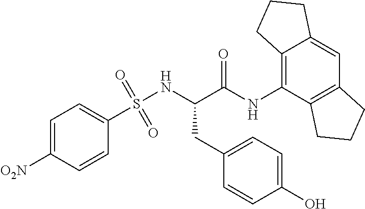 [SMILES: O=C(Nc1c2c(cc3c1CCC3)CCC2)[C@H](Cc1ccc(O)cc1)NS(=O)(=O)c1ccc([N+](=O)[O-])cc1]
(S)-6-amino-N-(1,2,3,5,6,7-hexahydro-s-indacen-4-yl)-2-((4-nitrophenyl)sulfonamido)hexanamide (9 of Formula (III)) 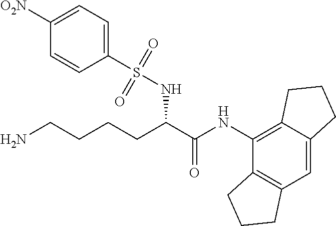 [SMILES: NCCCC[C@H](NS(=O)(=O)c1ccc([N+](=O)[O-])cc1)C(=O)Nc1c2c(cc3c1CCC3)CCC2]
N-(1,2,3,5,6,7-hexahydro-s-indacen-4-yl)-3-((4-nitrophenyl)sulfonamido)propanamide (6k of Formula (III)) 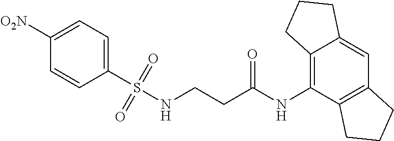 [SMILES: O=C(CCNS(=O)(=O)c1ccc([N+](=O)[O-])cc1)Nc1c2c(cc3c1CCC3)CCC2]
2-((4-aminophenyl)sulfonamido)-N-(1,2,3,5,6,7-hexahydro-s-indacen-4-yl)acetamide (7a of Formula (III)) 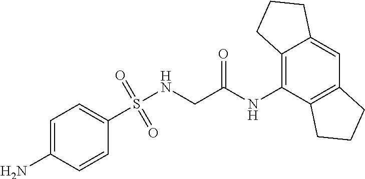 [SMILES: Nc1ccc(S(=O)(=O)NCC(=O)Nc2c3c(cc4c2CCC4)CCC3)cc1]
(S)-2-((4-aminophenyl)sulfonamido)-N-(1,2,3,5,6,7-hexahydro-s-indacen-4-yl)propanamide (7b of Formula (III)) 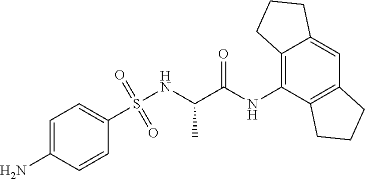 [SMILES: C[C@H](NS(=O)(=O)c1ccc(N)cc1)C(=O)Nc1c2c(cc3c1CCC3)CCC2]
(S)-2-((4-aminophenyl)sulfonamido)-N-(1,2,3,5,6,7-hexahydro-s-indacen-4-yl)-3-methylbutanamide (7c of Formula (III)) 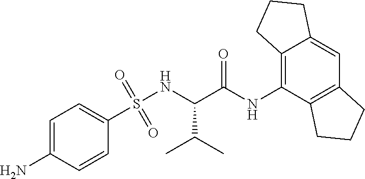 [SMILES: CC(C)[C@H](NS(=O)(=O)c1ccc(N)cc1)C(=O)Nc1c2c(cc3c1CCC3)CCC2]
(R)-2-((4-aminophenyl)sulfonamido)-N-(1,2,3,5,6,7-hexahydro-s-indacen-4-yl)-3-methylbutanamide (7 d of Formula (III)) 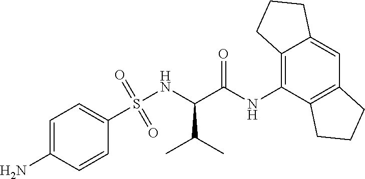 [SMILES: CC(C)[C@@H](NS(=O)(=O)c1ccc(N)cc1)C(=O)Nc1c2c(cc3c1CCC3)CCC2]
(S)-2-((4-aminophenyl)sulfonamido)-N-(1,2,3,5,6,7-hexahydro-s-indacen-4-yl)-4-methylpentanamide (7e of Formula (III)) 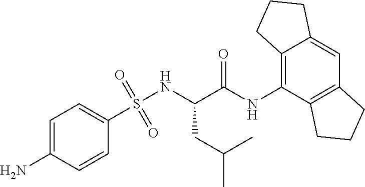 [SMILES: CC(C)C[C@H](NS(=O)(=O)c1ccc(N)cc1)C(=O)Nc1c2c(cc3c1CCC3)CCC2]
(S)-2-((4-aminophenyl)sulfonamido)-N-(1,2,3,5,6,7-hexahydro-s-indacen-4-yl)-2-phenylacetamide (7f of Formula (III)) 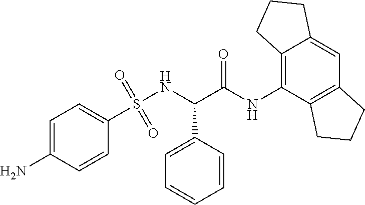 [SMILES: Nc1ccc(S(=O)(=O)N[C@H](C(=O)Nc2c3c(cc4c2CCC4)CCC3)c2ccccc2)cc1]
(S)-2-((4-aminophenyl)sulfonamido)-N-(1,2,3,5,6,7-hexahydro-s-indacen-4-yl)-3-phenylpropanamide (7g of Formula (III)) 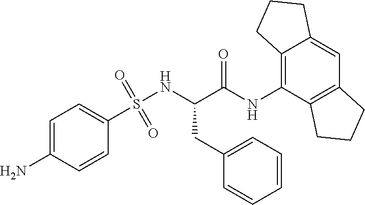 [SMILES: Nc1ccc(S(=O)(=O)N[C@@H](Cc2ccccc2)C(=O)Nc2c3c(cc4c2CCC4)CCC3)cc1]
(R)-2-((4-aminophenyl)sulfonamido)-N-(1,2,3,5,6,7-hexahydro-s-indacen-4-yl)-3-phenylpropanamide (7h of Formula (III)) 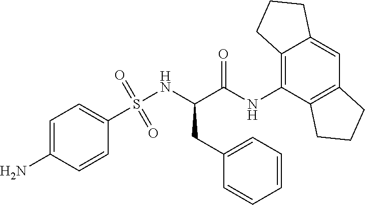 [SMILES: Nc1ccc(S(=O)(=O)N[C@H](Cc2ccccc2)C(=O)Nc2c3c(cc4c2CCC4)CCC3)cc1]
(S)-2-((4-aminophenyl)sulfonamido)-N-(1,2,3,5,6,7-hexahydro-s-indacen-4-yl)-3-(4-hydroxyphenyl)propanamide (10 of Formula (III)) 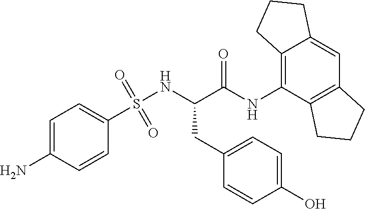 [SMILES: Nc1ccc(S(=O)(=O)N[C@@H](Cc2ccc(O)cc2)C(=O)Nc2c3c(cc4c2CCC4)CCC3)cc1]
(S)-6-amino-2-((4-aminophenyl)sulfonamido)-N-(1,2,3,5,6,7-hexahydro-s-indacen-4-yl)hexanamide (11 of Formula (III)) 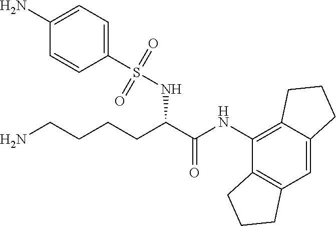 [SMILES: NCCCC[C@H](NS(=O)(=O)c1ccc(N)cc1)C(=O)Nc1c2c(cc3c1CCC3)CCC2]
3-((4-aminophenyl)sulfonamido)-N-(1,2,3,5,6,7-hexahydro-s-indacen-4-yl)propanamide (7k of Formula (III))

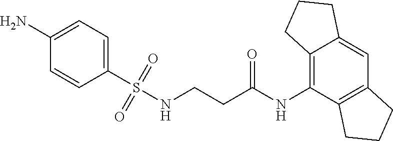 [SMILES: Nc1ccc(S(=O)(=O)NCCC(=O)Nc2c3c(cc4c2CCC4)CCC3)cc1]

19. The hexahydro-s-indacene compound of claim 1, wherein A is A3 and is a pyrrolidine/piperidine compound or its pharmaceutically acceptable salt of formula (IV): 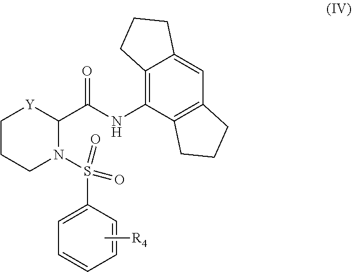
Y is —(CH₂)ₙ— and n is equal to 0 or 1; and
R₄ is a substituent selected from NH₂ and NO₂.

20. The hexahydro-s-indacene compound of claim 1, wherein when Y is equal to 1, R₄ is NO₂.

21. The hexahydro-s-indacene compound of claim 1, wherein when Y is equal to 0, R₄ is NH₂.

22. The hexahydro-s-indacene compound of claim 1, wherein the compound of Formula (I) is a pyrrolidine/piperidine compound of formula (IV) selected from the group consisting of:
N-(1,2,3,5,6,7-hexahydro-s-indacen-4-yl)-1-((4-nitrophenyl)sulfonyl)pyrrolidine-2-carboxamide (3a of Formula (IV)) 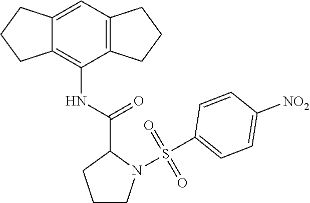 [SMILES: O=C(Nc1c2c(cc3c1CCC3)CCC2)C1CCCN1S(=O)(=O)c1ccc([N+](=O)[O-])cc1]
1-((4-aminophenyl)sulfonyl)-N-(1,2,3,5,6,7-hexahydro-s-indacen-4-yl)piperidine-2-carboxamide (4b of Formula (IV)) 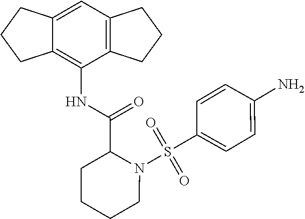 [SMILES: Nc1ccc(S(=O)(=O)N2CCCCC2C(=O)Nc2c3c(cc4c2CCC4)CCC3)cc1]
N-(1,2,3,5,6,7-hexahydro-s-indacen-4-yl)-1-((4-nitrophenyl)sulfonyl)piperidine-2-carboxamide (3b of Formula (IV)) 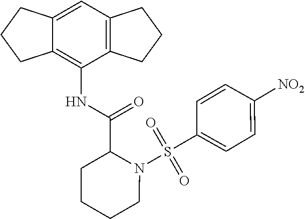 [SMILES: O=C(Nc1c2c(cc3c1CCC3)CCC2)C1CCCCN1S(=O)(=O)c1ccc([N+](=O)[O-])cc1]
1-((4-aminophenyl)sulfonyl)-N-(1,2,3,5,6,7-hexahydro-s-indacen-4-yl)pyrrolidine-2-carboxamide (4a of Formula (IV))

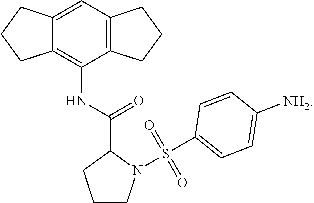 [SMILES: Nc1ccc(S(=O)(=O)N2CCCC2C(=O)Nc2c3c(cc4c2CCC4)CCC3)cc1]

23. (canceled)

24. A composition comprising a compound of formula (I) or a pharmaceutically acceptable salt thereof according to claim 1 and pharmaceutically acceptable additives.

25. A hexahydro-s-indacene compound of formula (I) or its pharmaceutically acceptable salt according to claim 1 for use as a selective inhibitor of the NLRP3 inflammasome.

26. The method of claim 25, wherein said pathologies are selected from the group consisting of cancer, metabolic disorders, neurodegenerative diseases, migraine, wound repair and autoimmune diseases.

27. A method for the treatment of pathologies related to hyperactivation of NLRP3 and hyperproduction of interleukin 1p and interleukin 18 comprising the step of administering the hexahydro-s-indacene compound of formula (I) or its pharmaceutically acceptable salt according to claim 1 as a selective inhibitor of the NLRP3 inflammasome.
